(19) United States
(12) Patent Application Publication
(10) Pub. No.: US 2022/0296627 A1
(43) Pub. Date: Sep. 22, 2022

(54) Cytidine Derivatives and Methods of Forming Cytidine Derivatives

(71) Applicant: Aenorasis Commercial Company of Pharmaceutical and Medical Products and Machines Societe Anonyme, Melissia (GR)

(72) Inventors: Andreas Tzakos, Ioannina (GR); Gregory Sivolapenko, Kifisia (GR); Christos Chatzigiannis, Petroupoli Attica (GR)

(21) Appl. No.: 17/605,794

(22) PCT Filed: Apr. 14, 2020
(86) PCT No.: PCT/EP2020/060405

§ 371 Date: Oct. 22, 2021

(30) Foreign Application Priority Data
Apr. 24, 2019 (GR) 20190100181

(51) Int. Cl. A61K 31/7068; A61P 35/00

(52) U.S. Cl. CPC A61K 31/7068; A61P 35/00

(57) ABSTRACT

Disclosed herein are nucleoside derivatives, cytidine derivatives and Gemcitabine derivatives and methods of forming nucleoside derivatives, cytidine derivatives and Gemcitabine derivatives.

### DESCRIPTION OF INVENTION

### FIELD OF THE INVENTION

The present disclosure relates to organic compounds, for example, nucleoside derivatives. The present disclosure also relates to methods of forming organic compounds, for example, nucleoside derivatives. In some aspects, the nucleoside derivatives are cytidine derivatives. In some aspects, the present disclosure relates to organic compounds, for example, derivatives of Gemcitabine (2′,2′-difluoro-2′-deoxycytidine) or any of its stereoisomers. In some aspects, the present disclosure relates to methods of forming derivatives of Gemcitabine (2′,2′-difluoro-2′-deoxycytidine) or any of its stereoisomers.

### BACKGROUND OF THE INVENTION

Gemcitabine (2′,2′-difluoro-2′-deoxycytidine) is a chemotherapy medication used to treat inter alia a number of different types of cancer. These cancers include breast cancer, ovarian cancer, non-small cell lung cancer, pancreatic cancer and bladder cancer. Gemcitabine belongs to the class of antimetabolites and is a nucleoside derivative of cytidine.

Although Gemcitabine has relatively high cytotoxicity, there are many factors that limit its therapeutic profile. The main limiting factors are its metabolic deamination at the 4-(N)-position by the enzyme cytidine deaminase (CDA) into its inactive uridine metabolite difluoro-deoxy-uridine (dFdU) and the lack of selectivity between cancer and normal cells. In order to overcome these obstacles, during standard clinical administration, Gemcitabine is administered in relatively high doses and as a result there are severe side effects.

Prodrugs of Gemcitabine have been synthesised in order to “protect” the 4-(N)-site of the molecule from deamination and two of these have reached clinical trials: LY2334737, an orally available valproic acid ester of Gemcitabine; and, Sq-Gemcitabine (SQdFdC) where squalene (an intermediate in cholesterol synthesis) is conjugated also at the 4-(N)-position.

The synthesis of prodrugs where the 4-(N)-position of Gemcitabine is chemically modified by selective groups requires a multi-step reaction (normally 4 steps, as shown in US20170107245A1 and WO2004041203A2, the contents of which are incorporated herein by reference) with low yields, high amounts of effluents and high cost.

There is a need to produce cytidine derivatives, for example Gemcitabine derivatives where the 4-(N) position (optionally only the 4-(N) position) is protected and/or derivatised. There is a need to provide more efficient methods of forming cytidine derivatives, for example Gemcitabine derivatives where the 4-(N) position (optionally only the 4-(N) position) is protected and/or derivatised.

### SUMMARY OF THE INVENTION

Representative features of the present invention are set out in the following clauses, which stand alone or may be combined, in any combination, with one or more features disclosed in the text and/or drawings of the specification.

1. A method for preparing 4-(N)-protected derivatives of compounds of formula (IB), or a pharmaceutically acceptable salt thereof, the method comprising:
reacting a compound of formula (IB):

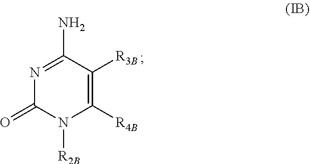

with an acyl chloride of the formula (II):

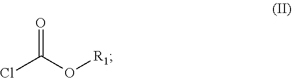

to produce a compound of the formula (IIIB):

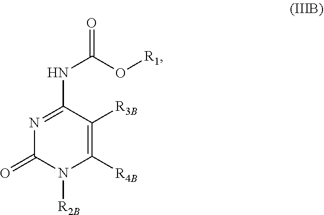

wherein:

R₁ is selected from the group consisting of: substituted or unsubstituted C₁-C₂₆ alkyl, substituted or unsubstituted C₁-C₂₆ haloalkyl, e.g. chloroalkyl, substituted or unsubstituted aryl, substituted or unsubstituted benzyl, substituted or unsubstituted C₂-C₂₆ alkenyl, substituted or unsubstituted C₂-C₂₆ alkynyl, C₁-C₂₆ alkyl substituted with one or more substituted or unsubstituted benzyl groups, C₁-C₂₆ alkyl substituted with one or more substituted or unsubstituted triazole groups;

R_{2B} is selected from the group consisting of: substituted or unsubstituted aromatic ring with 5 carbon atoms, substituted or unsubstituted aromatic ring with 6 carbon atoms, substituted or unsubstituted aryl, substituted or unsubstituted C₁-C₂₆ alkyl, substituted or unsubstituted α pyranose saccharide, substituted or unsubstituted β pyranose saccharide, substituted or unsubstituted a furanose saccharide, or substituted or unsubstituted β furanose saccharide;

R_{3B} is selected from the group consisting of: hydrogen, mono-substituted aromatic ring with 5 atoms, mono-substituted aromatic ring with 6 atoms, di-substituted aromatic ring with 5 atoms, di-substituted aromatic ring with 6 atoms, substituted or unsubstituted aryl, substituted or unsubstituted alkoxyalkane, carbonyl, halogen, substituted or unsubstituted C₁-C₂₆ alkyl, azide, substituted or unsubstituted C₂-C₂₆ alkynyl, substituted or unsubstituted C₂-C₂₆ alkenyl, hydroxyl, amino, or sulfur; and

R_{4B} is selected from the group consisting of: hydrogen, mono-substituted aromatic ring with 5 atoms, mono-substituted aromatic ring with 6 atoms, di-substituted aromatic ring with 5 atoms, di-substituted aromatic ring with 6 atoms, substituted or unsubstituted aryl, substituted or unsubstituted alkoxyalkane, carbonyl, halogen, substituted or unsubstituted C₁-C₂₆ alkyl, azide, substituted or unsubstituted C₂-C₂₆ alkynyl, substituted or unsubstituted C₂-C₂₆ alkenyl, hydroxyl, amino, or sulfur.

2. A method for preparing 4-(N)-protected derivatives of compounds of formula (IB), or a pharmaceutically acceptable salt thereof, the method comprising:
reacting a compound of formula (IB):

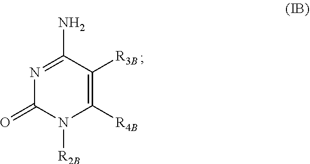

with a phosphoryl chloride of the formula (IIP):

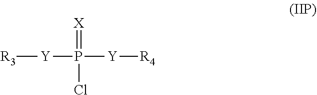

to produce a compound of the formula (IIIBP):

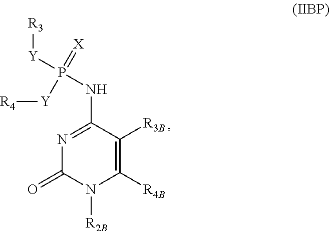

wherein,

R₃ and R₄ are both H; R₃ is H and R₄ is substituted or unsubstituted C₁-C₂₆ alkyl; or R₃ and R₄ are each independently substituted or unsubstituted C₁-C₂₆ alkyl;

X is O or S, particularly O;

each Y is independently O or S, and particularly each Y is O;

R_{2B} is selected from the group consisting of: substituted or unsubstituted aromatic ring with 5 carbon atoms, substituted or unsubstituted aromatic ring with 6 carbon atoms, substituted or unsubstituted aryl, substituted or unsubstituted C₁-C₂₆ alkyl, substituted or unsubstituted α pyranose saccharide, substituted or unsubstituted β pyranose saccharide, substituted or unsubstituted a furanose saccharide, or substituted or unsubstituted β furanose saccharide;

R_{3B} is selected from the group consisting of: hydrogen, mono-substituted aromatic ring with 5 atoms, mono-substituted aromatic ring with 6 atoms, di-substituted aromatic ring with 5 atoms, di-substituted aromatic ring with 6 atoms, substituted or unsubstituted aryl, substituted or unsubstituted alkoxyalkane, carbonyl, halogen, substituted or unsubstituted C₁-C₂₆ alkyl, azide, substituted or unsubstituted C₂-C₂₆ alkynyl, substituted or unsubstituted C₂-C₂₆ alkenyl, hydroxyl, amino, or sulfur; and

R_{4B} is selected from the group consisting of: hydrogen, mono-substituted aromatic ring with 5 atoms, mono-substituted aromatic ring with 6 atoms, di-substituted aromatic ring with 5 atoms, di-substituted aromatic ring with 6 atoms, substituted or unsubstituted aryl, substituted or unsubstituted alkoxyalkane, carbonyl, halogen, substituted or unsubstituted C₁-C₂₆ alkyl, azide, substituted or unsubstituted C₂-C₂₆ alkynyl, substituted or unsubstituted C₂-C₂₆ alkenyl, hydroxyl, amino, or sulfur.

3. The method of clause 1 or clause 2, wherein R_{2B} is selected from the group consisting of:

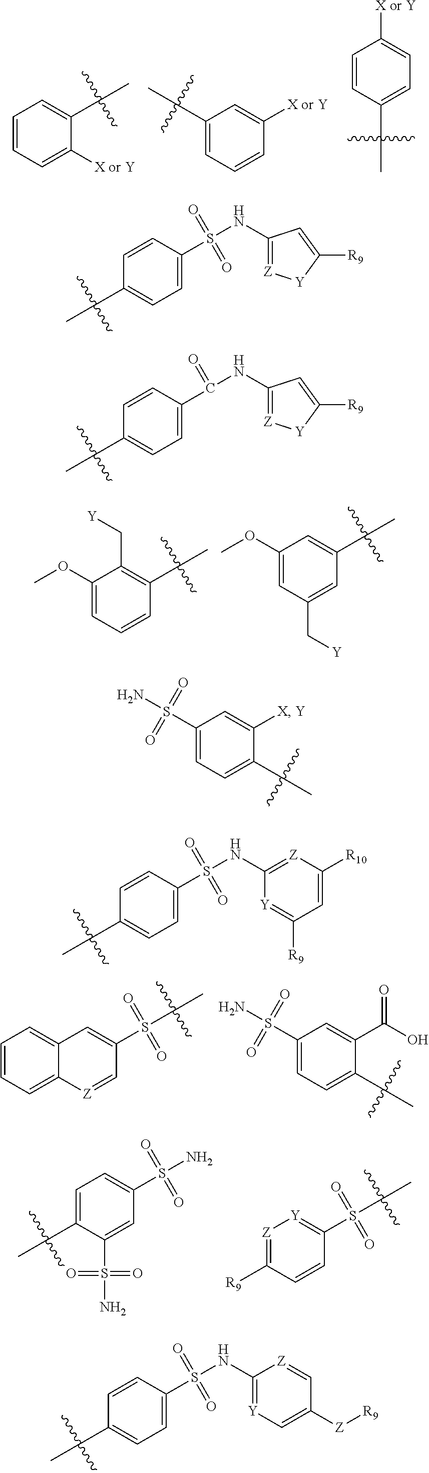

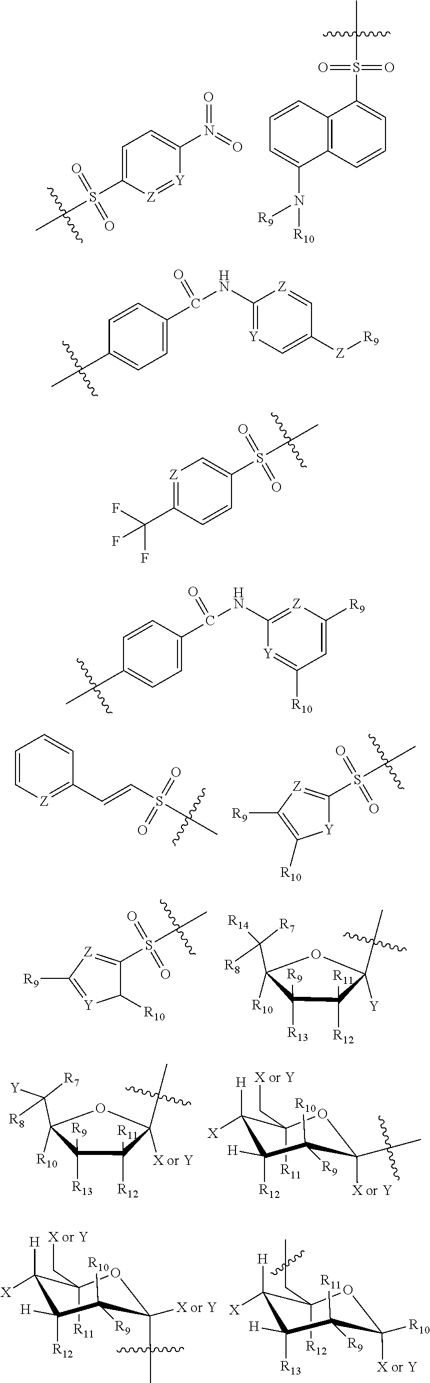

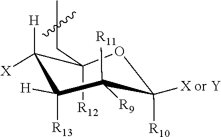

wherein:

the wavy line, at each incidence, shows the point of connection of R_{2B};

R₇ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;

R₈ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;

R₉ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;

R₁₀ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;

R₁₁ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;

R₁₂ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;

R₁₃ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;

R₁₄ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;

X is independently halogen;

Y is independently hydrogen, hydroxyl, amino or sulfur;

Z is independently hydroxyl, amino or sulfur.

4. The method of any one of clauses 1 to 3, wherein R_{3B} and R_{4B} are both hydrogen.

5. The method of any one of clauses 1 to 4, wherein the halogen at each incidence is independently F, Cl, Br or I.

6. The method of any one of clauses 1 to 5, wherein R_{3B} is hydrogen, R_{4B} is hydrogen, and R_{2B} is

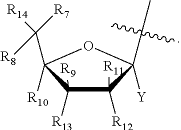

7. The method of clause 6, wherein Y is hydrogen, R₁₁ is halogen, R₁₂ is halogen, R₉ is hydrogen, R₁₃ is hydroxyl (—OH), R₁₀ is hydrogen, R₇ is hydrogen, R₈ is hydrogen and R₁₄ is hydroxyl (—OH).

8. A method for preparing 4-(N)-protected derivatives of Gemcitabine, or a pharmaceutically acceptable salt thereof, or the method of any one of clauses 1 or 3 to 7, the method comprising:
reacting Gemcitabine (I):

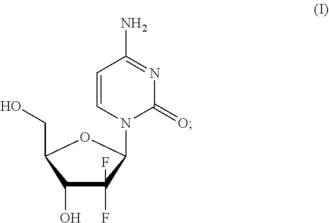

with an acyl chloride of the formula (II):

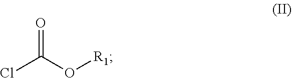

to produce a compound of the formula (III):

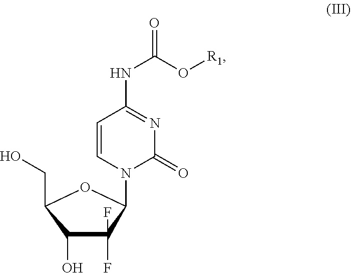

wherein R₁ is selected from the group consisting of: substituted or unsubstituted C₁-C₂₆ alkyl, substituted or unsubstituted C₁-C₂₆ haloalkyl, e.g. chloroalkyl, substituted or unsubstituted C₂-C₂₆ alkenyl, substituted or unsubstituted aryl, substituted or unsubstituted benzyl, substituted or unsubstituted C₂-C₂₆ alkynyl, C₁-C₂₆ alkyl substituted with one or more substituted or unsubstituted benzyl groups, C₁-C₂₆ alkyl substituted with one or more substituted or unsubstituted triazole groups.

9. The method of any one of clauses 1 to 5, wherein R_{3B} is halogen, R_{4B} is hydrogen, R₁ is —(CH₂)₄CH₃ and R_{2B} is

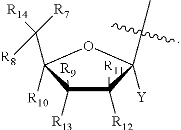

10. The method of clause 9, wherein R_{3B} is F.

11. The method of clause 9 or clause 10, wherein Y is hydrogen, R₁₁ is hydrogen, R₁₂ is hydroxyl (—OH), R₉ is hydrogen, R₁₃ is hydroxyl (—OH), R₁₀ is hydrogen, R₇ is hydrogen, R₈ is hydrogen and R₁₄ is hydrogen.

12. A method for preparing 4-(N)-protected derivatives of Gemcitabine, or a pharmaceutically acceptable salt thereof, or the method of any one of clauses 2 to 7 or 9 to 11, the method comprising:
reacting Gemcitabine (I):

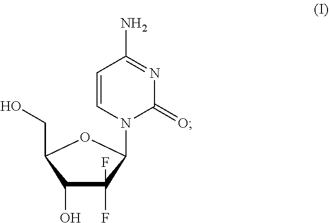

with a phosphoryl chloride of the formula (IIP):

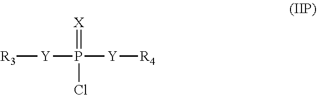

to produce a compound of the formula (IIIP):

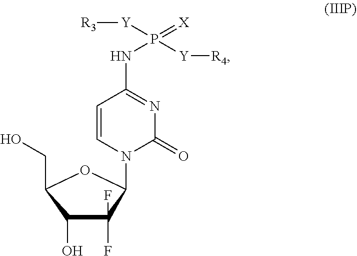

wherein,

R₃ and R₄ are both H; R₃ is H and R₄ is substituted or unsubstituted C₁-C₂₆ alkyl; or R₃ and R₄ are each independently substituted or unsubstituted C₁-C₂₆ alkyl;

X is O or S, particularly O; and

each Y is independently O or S, and particularly each Y is O.

13. The method of any one of clauses 1 to 12, wherein the method occurs in one pot; optionally, wherein the method occurs in a single step without isolation of an intermediate.

14. The method of any one of clauses 1 to 13, wherein the acyl chloride of the formula (II) or the phosphoryl chloride of the formula (IIP), is present in the method at from 0.3 to 0.7 equivalents (by moles).

15. The method of any one of clauses 1 to 14, wherein the acyl chloride of the formula (II) or the phosphoryl chloride of the formula (IIP), is present in the method at 0.5 equivalents (by moles).

16. The method of any one of clauses 1 to 15, wherein reacting the compound of formula (IB), optionally Gemcitabine (I), with the acyl chloride of formula (II) or the phosphoryl chloride of the formula (IIP) occurs in a solvent of ethyl acetate, acetyl cyanide or a mixture of ethyl acetate and acetyl cyanide.

17. The method of any one of clauses 1 to 16, wherein reacting the compound of formula (IB), optionally Gemcitabine (I), with the acyl chloride of formula (II) or the phosphoryl chloride of the formula (IIP) occurs under reflux conditions for from 1 to 4 hours; optionally for 3 hours; optionally, wherein reflux conditions occur at from 70° C. to 90° C., or at 80° C.

18. The method of any one of clauses 1 to 17, wherein R₁ is selected from the group consisting of:

i. —CH₂CH₃, —(CH₂)₂CH₃, —(CH₂)₃CH₃, —(CH₂)₄CH₃, —(CH₂)₅CH₃, —(CH₂)₆CH₃, —CH₂CH(CH₃)₂, —(CH₂)₂CH(CH₃)₂, —(CH₂)₃CH(CH₃)₂, —(CH₂)₄CH(CH₃)₂;

ii. —CH₂Cl, —(CH₂)₂Cl, —(CH₂)₃Cl, —(CH₂)₄Cl, —(CH₂)₅Cl, —(CH₂)₆Cl, —CH₂Br, —(CH₂)₂Br, —(CH₂)₃Br, —(CH₂)₄Br, —(CH₂)₅Br, —(CH₂)₆Br, —CH₂I, —(CH₂)₂I, —(CH₂)₃I, —(CH₂)₄I, —(CH₂)₅I, —(CH₂)₆I;

iii. —CH₂CCH, —(CH₂)₂CCH, —(CH₂)₃CCH, —(CH₂)₄CCH, —(CH₂)₅CCH, —(CH₂)₆CCH,

iv. —CH₂N₃, —(CH₂)₂N₃, —(CH₂)₃N₃, —(CH₂)₄N₃, —(CH₂)₅N₃, —(CH₂)₆N₃,

v. —CH₂SH, —(CH₂)₂SH, —(CH₂)₃SH, —(CH₂)₄SH, —(CH₂)₅SH, —(CH₂)₆SH,

vi. —CH₂COOH, —(CH₂)₂COOH, —(CH₂)₃COOH, —(CH₂)₄COOH, —(CH₂)₅COOH, —(CH₂)₆COOH, —CH₂COOR₂, —(CH₂)₂COOR₂, —(CH₂)₃COOR₂, —(CH₂)₄COOR₂, —(CH₂)₅COOR₂, —(CH₂)₆COOR₂;

vii. —CH₂Ar, —(CH₂)₂Ar, —(CH₂)₃Ar, —(CH₂)₄Ar, —(CH₂)₅Ar, —(CH₂)₆Ar, −CH₂CHArCH₃, —CH₂CHArCH₂CH₃;

viii. —CH₂Tr, —(CH₂)₂Tr, —(CH₂)₃Tr, —(CH₂)₄Tr, —(CH₂)₅Tr, —(CH₂)₆Tr, —CH₂CHTrCH₃ or —CH₂CHTrCH₂CH₃;

wherein R₂ is substituted or unsubstituted C₁-C₂₆ alkyl;
wherein Ar is

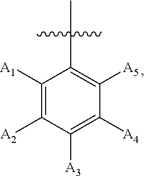

wherein A₁, A₂, A₃, A₄ and A₅ are each independently H, NO₂, OH, O-alkyl or O-methyl; optionally, wherein A₁ is NO₂ and A₂, A₃, A₄ and A₅ are H; or, wherein A₁ is NO₂, A₃ and A₄ are O-methyl and A₂ and A₅ are H; and/or,
wherein Tr is N

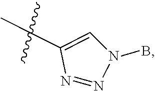

wherein B is substituted or unsubstituted alkyl, substituted or unsubstituted haloalkyl, e.g. chloroalkyl, substituted or unsubstituted aryl, substituted or unsubstituted benzyl, substituted or unsubstituted alkenyl, substituted or unsubstituted alkynyl, alkyl substituted with one or more benzyl or substituted benzyl groups or

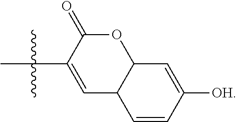 [SMILES: *C1=CC2C=CC(O)=CC2OC1=O]

Optionally, R₁ is not as set out in any one or more of clauses 18.i., 18.ii., 18.iii., 18.iv., 18.v., 18.vi., 18.vii., or 18.viii.

19. The method of any one of clauses 1 to 18, wherein R₁ comprises a substituent reactive with the H atom on 4-(N), e.g. wherein R₁ is chloroalkyl and the method further comprises the step of reacting the compound of the formula (III):

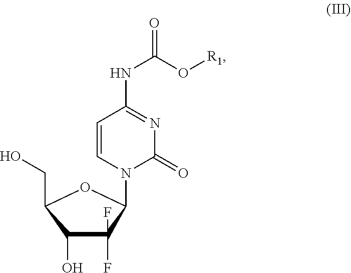

in a solvent, e.g. N,N-diisopropylethylamine, under suitable conditions, e.g. reflux conditions, to form a compound of formula (IV):

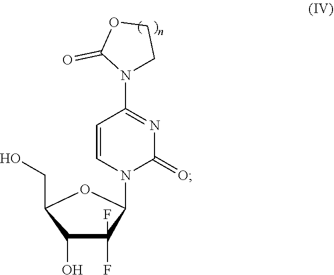

wherein n is 0, 1 or 2.

20. The method of any one of clauses 1 to 19, wherein the method further comprises the step of reacting the compound of the formula (III) or (IIIP) with an OH-reactive derivatising agent to form a 3′- and/or 5′-substituted derivative of compound (III) or (IIIP);

optionally, wherein the method further comprises the step of reacting the compound of formula (III) with acetic anhydride to form a compound of the formula (V):

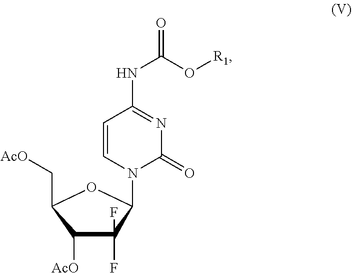

or, formula (VP):

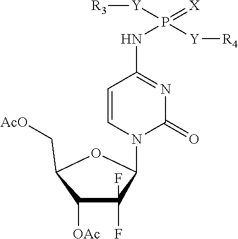

wherein Ac is —COCH₃.

21. A compound obtainable by, or obtained from, the method of any one of clauses 1 to 20.

22. A compound of the formula (III), or a 3′- and/or 5′-substituted derivative thereof, for example a compound of formula (VA) or (V):

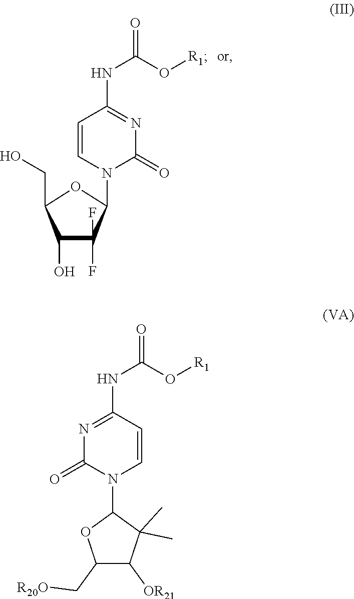

wherein at least one of R₂₀ and R₂₁ is not H, and,

R₂₀ is H or —COR₂₀₁ where R₂₀₁ is selected from the group consisting of: substituted or unsubstituted C₁-C₂₆ alkyl, substituted or unsubstituted C₁-C₂₆ haloalkyl, e.g. chloroalkyl, substituted or unsubstituted aryl, substituted or unsubstituted benzyl, substituted or unsubstituted C₂-C₂₆ alkenyl, substituted or unsubstituted C₂-C₂₆ alkynyl, C₁-C₂₆ alkyl substituted with one or more substituted or unsubstituted benzyl groups, C₁-C₂₆ alkyl substituted with one or more substituted or unsubstituted triazole groups; and,

R₂₁ is H or —COR₂₀₂ where R₂₀₂ is selected from the group consisting of: substituted or unsubstituted C₁-C₂₆ alkyl, substituted or unsubstituted C₁-C₂₆ haloalkyl, e.g. chloroalkyl, substituted or unsubstituted aryl, substituted or unsubstituted benzyl, substituted or unsubstituted C₂-C₂₆ alkenyl, substituted or unsubstituted C₂-C₂₆ alkynyl, C₁-C₂₆ alkyl substituted with one or more substituted or unsubstituted benzyl groups, C₁-C₂₆ alkyl substituted with one or more substituted or unsubstituted triazole groups; or,

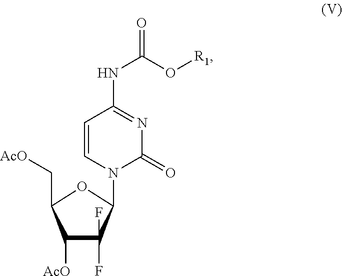

wherein Ac is —COCH₃;

wherein R₁ is selected from the group consisting of: substituted or unsubstituted C₁-C₂₆ alkyl, substituted or unsubstituted C₁-C₂₆ haloalkyl, e.g. chloroalkyl, substituted or unsubstituted aryl, substituted or unsubstituted benzyl, substituted or unsubstituted C₂-C₂₆ alkenyl, substituted or unsubstituted C₂-C₂₆ alkynyl, C₁-C₂₆ alkyl substituted with one or more substituted or unsubstituted benzyl groups, C₁-C₂₆ alkyl substituted with one or more substituted or unsubstituted triazole groups;
or a pharmaceutically acceptable salt thereof.

23. The compound of clause 22, wherein R₁ is selected from the group consisting of: —CH₂CH₃, —(CH₂)₂CH₃, —(CH₂)₃CH₃, —(CH₂)₄CH₃, —(CH₂)₅CH₃, —(CH₂)₆CH₃, —CH₂CH(CH₃)₂, —(CH₂)₂CH(CH₃)₂, —(CH₂)₃CH(CH₃)₂ or —(CH₂)₄CH(CH₃)₂.

24. The compound of clause 22, wherein R₁ is selected from the group consisting of: —CH₂Cl, —(CH₂)₂Cl, —(CH₂)₃Cl, —(CH₂)₄Cl, —(CH₂)₅Cl, —(CH₂)₆Cl, —CH₂Br, —(CH₂)₂Br, —(CH₂)₃Br, —(CH₂)₄Br, —(CH₂)₅Br, —(CH₂)₆Br, —CH₂I, —(CH₂)₂I, —(CH₂)₃I, —(CH₂)₄I, —(CH₂)₅I or —(CH₂)₆I.

25. The compound of clause 22, wherein R₁ is selected from the group consisting of: —CH₂CCH, —(CH₂)₂CCH, —(CH₂)₃CCH, —(CH₂)₄CCH, —(CH₂)₅CCH or —(CH₂)₆CCH.

26. The compound of clause 22, wherein R₁ is selected from the group consisting of: —CH₂N₃, —(CH₂)₂N₃, —(CH₂)₃N₃, —(CH₂)₄N₃, —(CH₂)₅N₃ or —(CH₂)₆N₃.

27. The compound of clause 22, wherein R₁ is selected from the group consisting of: —CH₂SH, —(CH₂)₂SH, —(CH₂)₃SH, —(CH₂)₄SH, —(CH₂)₅SH or —(CH₂)₆SH.

28. The compound of clause 22, wherein R₁ is selected from the group consisting of: —CH₂COOH, —(CH₂)₂COOH, —(CH₂)₃COOH, —(CH₂)₄COOH, —(CH₂)₅COOH, —(CH₂)₆COOH, —CH₂COOR₂, —(CH₂)₂COOR₂, —(CH₂)₃COOR₂, —(CH₂)₄COOR₂, —(CH₂)₅COOR₂ or —(CH₂)₆COOR₂;
wherein R₂ is substituted or unsubstituted C₁-C₂₆ alkyl.

29. The compound of clause 22, wherein R₁ is selected from the group consisting of: —CH₂Ar, —(CH₂)₂Ar, —(CH₂)₃Ar, —(CH₂)₄Ar, —(CH₂)₅Ar, —(CH₂)₆Ar, —CH₂CHArCH₃ or —CH₂CHArCH₂CH₃;
wherein Ar is

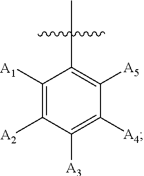

wherein A₁, A₂, A₃, A₄ and A₅ are each independently H, NO₂, OH, O-alkyl or O-methyl; optionally, wherein A₁ is NO₂ and A₂, A₃, A₄ and A₅ are H; or, wherein A₁ is NO₂, A₃ and A₄ are O-methyl and A₂ and A₅ are H.

30. The compound of clause 22, wherein R₁ is selected from the group consisting of: —CH₂Tr, —(CH₂)₂Tr, —(CH₂)₃Tr, —(CH₂)₄Tr, —(CH₂)₅Tr, —(CH₂)₆Tr, —CH₂CHTrCH₃ or —CH₂CHTrCH₂CH₃;
wherein Tr is

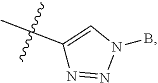

wherein B is substituted or unsubstituted C₁-C₂₆ alkyl, substituted or unsubstituted C₁-C₂₆ haloalkyl, e.g. chloroalkyl, substituted or unsubstituted aryl, substituted or unsubstituted benzyl, substituted or unsubstituted C₂-C₂₆ alkenyl, substituted or unsubstituted C₂-C₂₆ alkynyl, C₁-C₂₆ alkyl substituted with one or more benzyl or substituted benzyl groups, or,

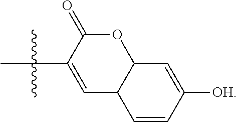 [SMILES: *C1=CC2C=CC(O)=CC2OC1=O]

Optionally, R₁ is not as set out in any one or more of clauses 23, 24, 25, 26, 27, 28, 29 or 30.

31. The compound of any one of clauses 22 to 30, wherein the compound is selected from the group consisting of:

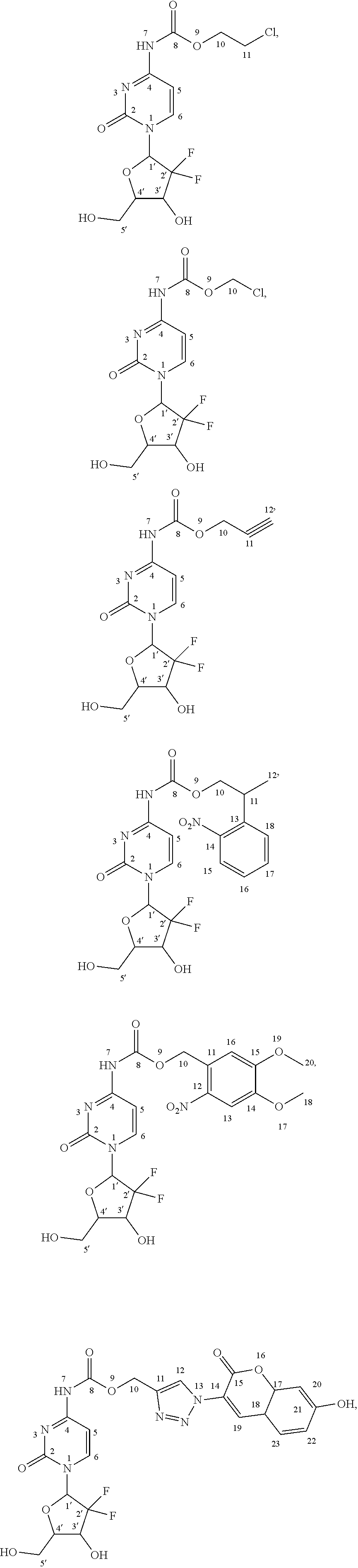

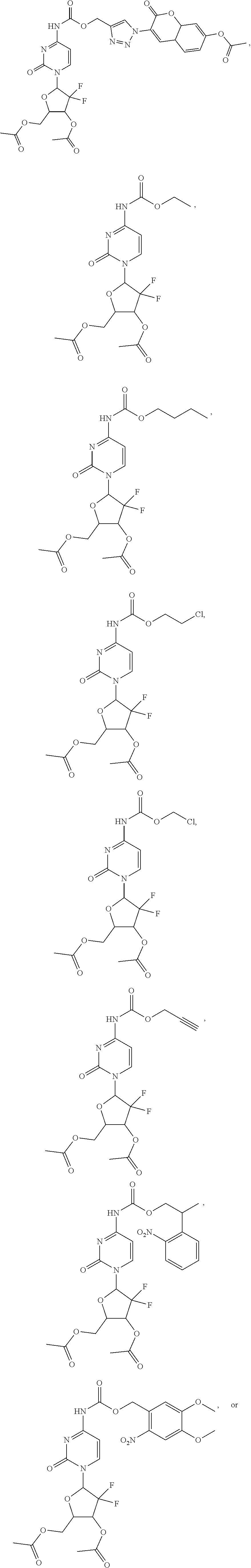

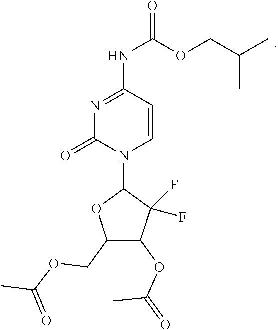 [SMILES: CC(=O)OCC1OC(n2ccc(NC(=O)OCC(C)C)nc2=O)C(F)(F)C1OC(C)=O]

32. The compound of any one of clauses 22 to 30, wherein the compound is not selected from the group consisting of:

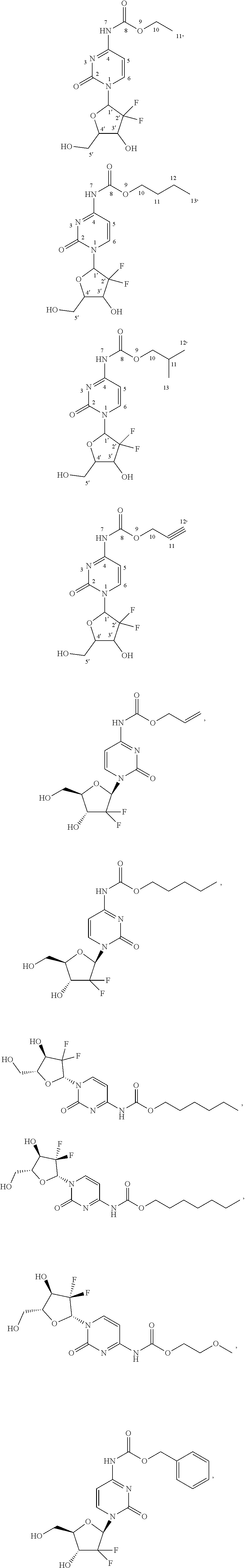

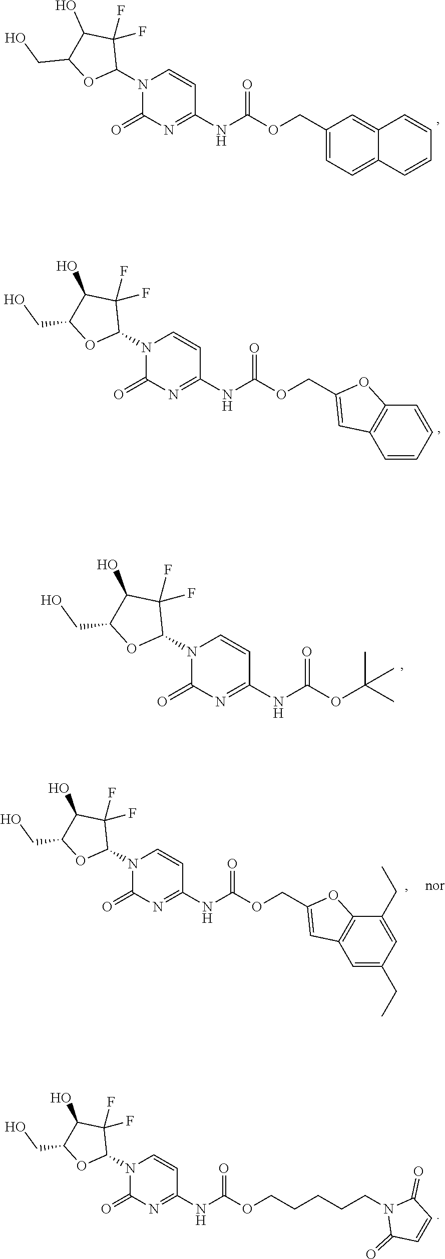 [SMILES: O=C(Nc1ccn(C2OC(CO)C(O)C2(F)F)c(=O)n1)OCc1ccc2ccccc2c1 ; O=C(Nc1ccn([C@@H]2O[C@H](CO)[C@@H](O)C2(F)F)c(=O)n1)OCc1cc2ccccc2o1 ; CC(C)(C)OC(=O)Nc1ccn([C@@H]2O[C@H](CO)[C@@H](O)C2(F)F)c(=O)n1 ; CCc1cc(CC)c2oc(COC(=O)Nc3ccn([C@@H]4O[C@H](CO)[C@@H](O)C4(F)F)c(=O)n3)cc2c1 ; O=C(Nc1ccn([C@@H]2O[C@H](CO)[C@@H](O)C2(F)F)c(=O)n1)OCCCCCN1C(=O)C=CC1=O]

33. A compound of the formula (IIIP) or a 3′- and/or 5′-substituted derivative thereof:

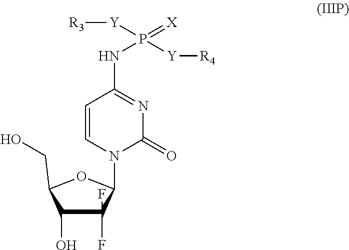

wherein R₃ and R₄ are both H; R₃ is H and R₄ is substituted or unsubstituted C₁-C₂₆ alkyl; or R₃ and R₄ are each independently substituted or unsubstituted C₁-C₂₆ alkyl;

X is O or S, particularly O; and

each Y is independently O or S, and particularly each Y is O;

or a pharmaceutically acceptable salt thereof.

34. A compound according to clause 33, of the formula (VI) or a 3′-and/or 5′-substituted derivative thereof:

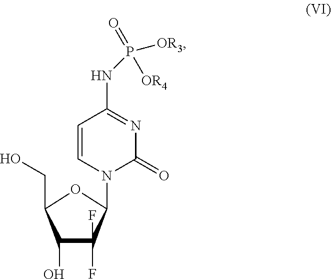

wherein:
   R₃ and R₄ are both H;
   R₃ is H and R₄ is substituted or unsubstituted C₁-C₂₆ alkyl; or,
   R₃ and R₄ are each independently substituted or unsubstituted C₁-C₂₆ alkyl;
or a pharmaceutically acceptable salt thereof.

35. The compound of clause 33 or clause 34, wherein one or both of R₃ and R₄ is selected from the group consisting of: —CH₂CH₃, —(CH₂)₂CH₃, —(CH₂)₃CH₃, —(CH₂)₄CH₃, —(CH₂)₅CH₃, —(CH₂)₆CH₃, —CH₂CH(CH₃)₂, —(CH₂)₂CH(CH₃)₂, —(CH₂)₃CH(CH₃)₂, or —(CH₂)₄CH(CH₃)₂.

36. A compound of formula (IV):

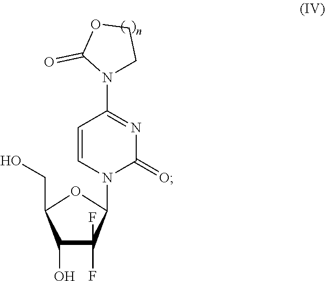

wherein n is 0, 1 or 2;
or a pharmaceutically acceptable salt thereof.

37. A pharmaceutical composition comprising a compound according to any one of clauses 21 to 36 and a pharmaceutically acceptable carrier.

38. A compound according to any one of clauses 21 to 36, or a pharmaceutical composition according to clause 37, for use in therapy.

39. A compound according to any one of clauses 21 to 36, or a pharmaceutical composition according to clause 37, for use in treating cancer.

40. The compound or pharmaceutical composition for use according to clause 39, wherein the cancer is selected from the group consisting of: breast cancer, ovarian cancer, non-small cell lung cancer, pancreatic cancer and bladder cancer.

41. A method of treating a patient, optionally a human patient, suffering from cancer, the method comprising administering an effective amount of a compound according to any one of clauses 21 to 36, or a pharmaceutical composition according to clause 37, to the patient.

42. The method of clause 41, wherein the cancer is selected from the group consisting of: breast cancer, ovarian cancer, non-small cell lung cancer, pancreatic cancer and bladder cancer.

Some further aspects of the present invention are disclosed with reference to the following clauses (“A” clauses), which stand alone or may be combined, in any combination, with one or more features disclosed in the text and/or drawings of the specification.

1A. A method for preparing 4-(N)-protected derivatives of Gemcitabine, or a pharmaceutically acceptable salt thereof, the method comprising:
reacting Gemcitabine (I):

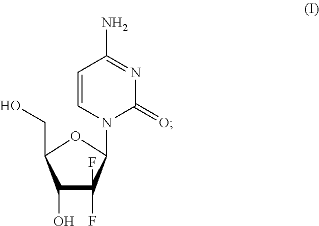

with an acyl chloride of the formula (II):

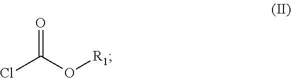

to produce a compound of the formula (III):

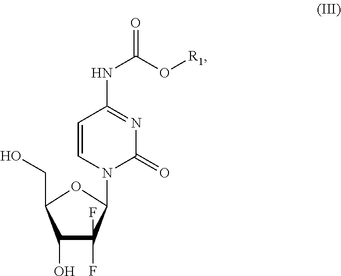

wherein R₁ is selected from the group consisting of: substituted or unsubstituted C₁-C₂₆ alkyl, substituted or unsubstituted C₁-C₂₆ chloroalkyl, substituted or unsubstituted aryl, substituted or unsubstituted benzyl, substituted or unsubstituted C₁-C₂₆ alkynyl, C₁-C₂₆ alkyl substituted with one or more substituted or unsubstituted benzyl groups, C₁-C₂₆ alkyl substituted with one or more substituted or unsubstituted triazole groups.

2A. The method of clause 1A, wherein the method occurs in one pot.

3A. The method of clause 1A or clause 2A, wherein the acyl chloride of the formula (II):

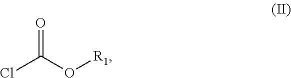

is present in the method at from 0.3 to 0.7 equivalents (by moles).

4A. The method of any one of clauses 1A to 3A, wherein the acyl chloride of the formula (II):

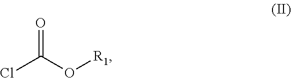

is present in the method at 0.5 equivalents (by moles).

5A. The method of any one of clauses 1A to 4A, wherein reacting Gemcitabine (I) with an acyl chloride of formula (II) occurs in a solvent of ethyl acetate, acetyl cyanide or a mixture of ethyl acetate and acetyl cyanide.

6A. The method of any one of clauses 1A to 5A, wherein reacting Gemcitabine (I) with an acyl chloride of formula (II) occurs under reflux conditions for from 1 to 4 hours; optionally for 3 hours; optionally, wherein reflux conditions occur at from 70° C. to 90° C., or at 80° C.

7A. The method of any one of clauses 1A to 6A, wherein R₁ is selected from the group consisting of: —CH₂CH₃, —(CH₂)₂CH₃, —(CH₂)₃CH₃, —(CH₂)₄CH₃, —(CH₂)₅CH₃, —(CH₂)₆CH₃, —CH₂CH(CH₃)₂, —(CH₂)₂CH(CH₃)₂, —(CH₂)₃CH(CH₃)₂, —(CH₂)₄CH(CH₃)₂, —CH₂Cl, —(CH₂)₂Cl, —(CH₂)₃Cl, —(CH₂)₄Cl, —(CH₂)₅Cl, —(CH₂)₆Cl, —CH₂Br, —(CH₂)₂Br, —(CH₂)₃Br, —(CH₂)₄Br, —(CH₂)₅Br, —(CH₂)₆Br, —CH₂I, —(CH₂)₂I, —(CH₂)₃I, —(CH₂)₄I, —(CH₂)₅I, —(CH₂)₆I, —CH₂CCH, —(CH₂)₂CCH, —(CH₂)₃CCH, —(CH₂)₄CCH, —(CH₂)₅CCH, —(CH₂)₆CCH, —CH₂N₃, —(CH₂)₂N₃, —(CH₂)₃N₃, —(CH₂)₄N₃, —(CH₂)₅N₃, —(CH₂)₆N₃, —CH₂SH, —(CH₂)₂SH, —(CH₂)₃SH, —(CH₂)₄SH, —(CH₂)₅SH, —(CH₂)₆SH, —CH₂COOH, —(CH₂)₂COOH, —(CH₂)₃COOH, —(CH₂)₄COOH, —(CH₂)₅COOH, —(CH₂)₆COOH, —CH₂COOR₂, —(CH₂)₂COOR₂, —(CH₂)₃COOR₂, —(CH₂)₄COOR₂, —(CH₂)₅COOR₂, —(CH₂)₆COOR₂, —CH₂Ar, —(CH₂)₂Ar, —(CH₂)₃Ar, —(CH₂)₄Ar, —(CH₂)₅Ar, —(CH₂)₆Ar, —CH₂CHArCH₃, —CH₂CHArCH₂CH₃, —CH₂Tr, —(CH₂)₂Tr, —(CH₂)₃Tr, —(CH₂)₄Tr, —(CH₂)₅Tr, —(CH₂)₆Tr, —CH₂CHTrCH₃ or —CH₂CHTrCH₂CH₃;
wherein R₂ is substituted or unsubstituted C₁-C₂₆ alkyl;
wherein Ar is

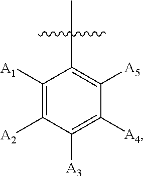

wherein A₁, A₂, A₃, A₄ and A₅ are each independently H, NO₂, OH, O-alkyl or O-methyl; optionally, wherein A₁ is NO₂ and A₂, A₃, A₄ and A₅ are H; or, wherein A₁ is NO₂, A₃ and A₄ are OMe and A₂ and A₅ are H; or,

wherein Tr is

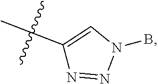

wherein B is substituted or unsubstituted alkyl, substituted or unsubstituted chloroalkyl, substituted or unsubstituted aryl, substituted or unsubstituted benzyl, substituted or unsubstituted alkynyl, alkyl substituted with one or more benzyl or substituted benzyl groups or

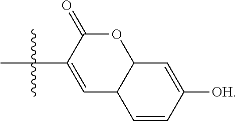 [SMILES: *C1=CC2C=CC(O)=CC2OC1=O]

8A. The method of any one of clauses 1A to 4A, wherein R₁ is chloroalkyl and the method further comprises the step of reacting the compound of the formula (III):

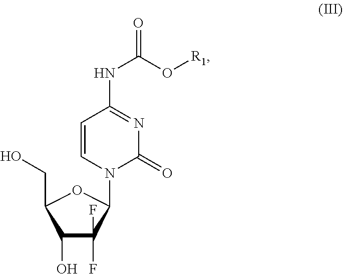

in N,N-diisopropylethylamine under reflux conditions to form a compound of formula (IV):

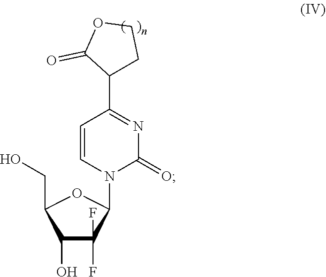

wherein n is 0, 1 or 2.

9A. The method of any one of clauses 1A to 8A, wherein the method further comprises the step of reacting the compound of the formula (III):

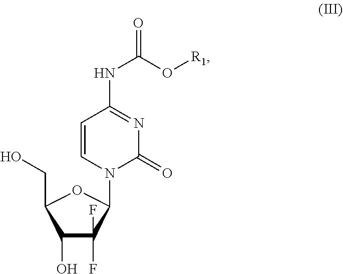

with acetic anhydride to form a compound of the formula (V):

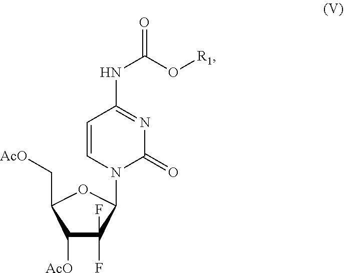

wherein Ac is —COCH₃.

10A. A compound obtainable by, or obtained from, the method of any one of clauses 1A to 9A.

11A. A compound of the formula (III) or formula (V):

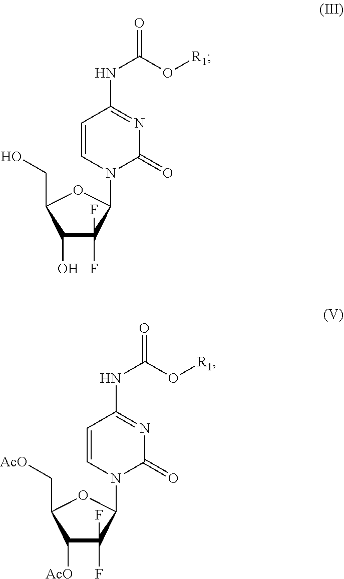

(V), wherein Ac is —COCH₃;

wherein R₁ is selected from the group consisting of: substituted or unsubstituted C₁-C₂₆ alkyl, substituted or unsubstituted C₁-C₂₆ chloroalkyl, substituted or unsubstituted aryl, substituted or unsubstituted benzyl, substituted or unsubstituted C₁-C₂₆ alkynyl, C₁-C₂₆ alkyl substituted with one or more substituted or unsubstituted benzyl groups, C₁-C₂₆ alkyl substituted with one or more substituted or unsubstituted triazole groups;
or a pharmaceutically acceptable salt thereof.

12A. The compound of clause 11A, wherein R₁ is selected from the group consisting of: —CH₂CH₃, —(CH₂)₂CH₃, —(CH₂)₃CH₃, —(CH₂)₄CH₃, —(CH₂)₅CH₃, —(CH₂)₆CH₃, —CH₂CH(CH₃)₂, —(CH₂)₂CH(CH₃)₂, —(CH₂)₃CH(CH₃)₂ or —(CH₂)₄CH(CH₃)₂.

13A. The compound of clause 11A, wherein R₁ is selected from the group consisting of: —CH₂Cl, —(CH₂)₂Cl, —(CH₂)₃Cl, —(CH₂)₄Cl, —(CH₂)₅Cl, —(CH₂)₆Cl, —CH₂Br, —(CH₂)₂Br, —(CH₂)₃Br, —(CH₂)₄Br, —(CH₂)₅Br, —(CH₂)₆Br, —CH₂I, —(CH₂)₂I, —(CH₂)₃I, —(CH₂)₄I, —(CH₂)₅I or —(CH₂)₆I,

14A. The compound of clause 11A, wherein R₁ is selected from the group consisting of: —CH₂CCH, —(CH₂)₂CCH, —(CH₂)₃CCH, —(CH₂)₄CCH, —(CH₂)₅CCH or —(CH₂)₆CCH.

15A. The compound of clause 11A, wherein R₁ is selected from the group consisting of: —CH₂N₃, —(CH₂)₂N₃, —(CH₂)₃N₃, —(CH₂)₄N₃, —(CH₂)₅N₃ or —(CH₂)₆N₃.

16A. The compound of clause 11A, wherein R₁ is selected from the group consisting of: —CH₂SH, —(CH₂)₂SH, —(CH₂)₃SH, —(CH₂)₄SH, —(CH₂)₅SH or —(CH₂)₆SH.

17A. The compound of clause 11A, wherein R₁ is selected from the group consisting of: —CH₂COOH, —(CH₂)₂COOH, —(CH₂)₃COOH, —(CH₂)₄COOH, —(CH₂)₅COOH, —(CH₂)₆COOH, —CH₂COOR₂, —(CH₂)₂COOR₂, —(CH₂)₃COOR₂, —(CH₂)₄COOR₂, —(CH₂)₅COOR₂ or —(CH₂)₆COOR₂;
wherein R₂ is substituted or unsubstituted C₁-C₂₆ alkyl.

18A. The compound of clause 11A, wherein R₁ is selected from the group consisting of: —CH₂Ar, —(CH₂)₂Ar, —(CH₂)₃Ar, —(CH₂)₄Ar, —(CH₂)₅Ar, —(CH₂)₆Ar, —CH₂CHArCH₃ or —CH₂CHArCH₂CH₃;
wherein Ar is

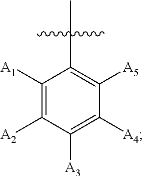

wherein A₁, A₂, A₃, A₄ and A₅ are each independently H, NO₂, OH, O-alkyl or O-methyl; optionally, wherein A₁ is NO₂ and A₂, A₃, A₄ and A₅ are H; or, wherein A₁ is NO₂, A₃ and A₄ are OMe and A₂ and A₅ are H.

19A. The compound of clause 11A, wherein R₁ is selected from the group consisting of: —CH₂Tr, —(CH₂)₂Tr, —(CH₂)₃Tr, —(CH₂)₄Tr, —(CH₂)₅Tr, —(CH₂)₆Tr, —CH₂CHTrCH₃ or —CH₂CHTrCH₂CH₃;

wherein Tr is

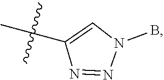

wherein B is substituted or unsubstituted C₁-C₂₆ alkyl, substituted or unsubstituted C₁-C₂₆ chloroalkyl, substituted or unsubstituted aryl, substituted or unsubstituted benzyl, substituted or unsubstituted C₁-C₂₆ alkynyl, C₁-C₂₆ alkyl substituted with one or more benzyl or substituted benzyl groups, or,

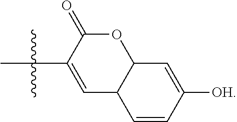 [SMILES: *C1=CC2C=CC(O)=CC2OC1=O]

Optionally, R₁ is not as set out in any one or more of clauses 12A, 13A, 14A, 15A, 16A, 17A, 18A or 19A.

20A. The compound of any one of clauses 11A to 19A, wherein the compound is selected from the group consisting of:

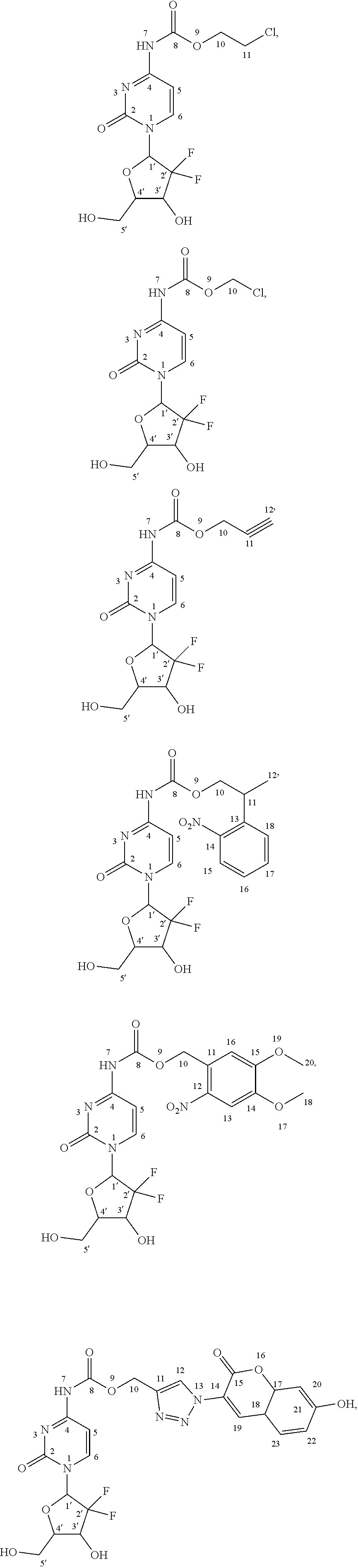

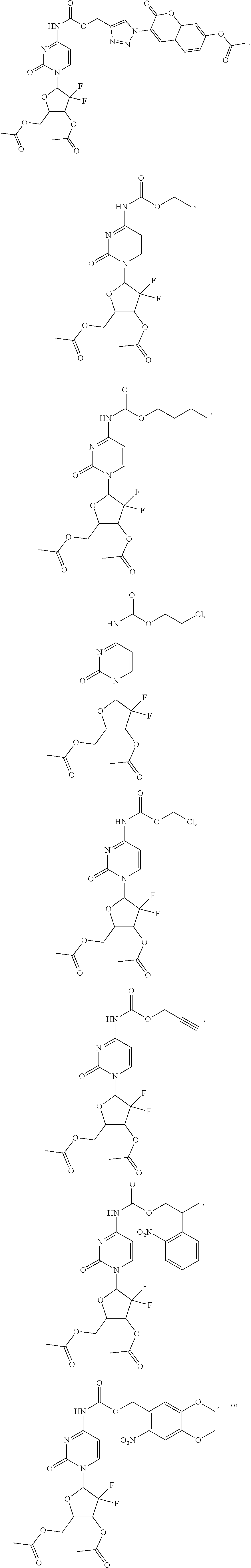

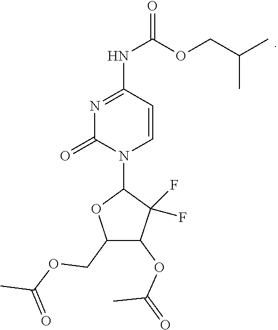 [SMILES: CC(=O)OCC1OC(n2ccc(NC(=O)OCC(C)C)nc2=O)C(F)(F)C1OC(C)=O]

21A. The compound of any one of clauses 11A to 19A, wherein the compound is not selected from the group consisting of:

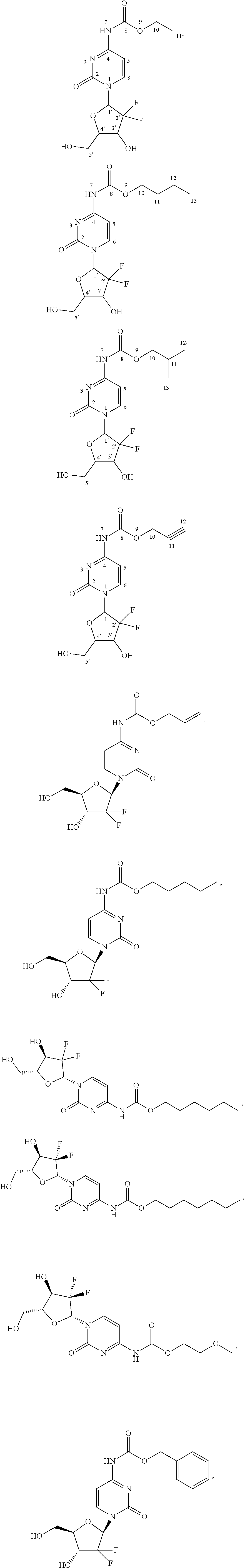

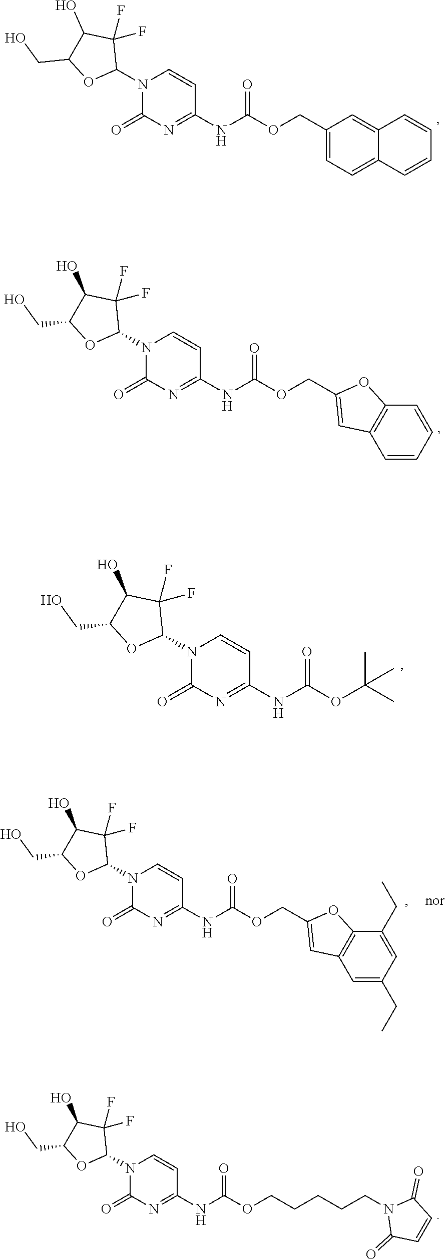 [SMILES: O=C(Nc1ccn(C2OC(CO)C(O)C2(F)F)c(=O)n1)OCc1ccc2ccccc2c1 ; O=C(Nc1ccn([C@@H]2O[C@H](CO)[C@@H](O)C2(F)F)c(=O)n1)OCc1cc2ccccc2o1 ; CC(C)(C)OC(=O)Nc1ccn([C@@H]2O[C@H](CO)[C@@H](O)C2(F)F)c(=O)n1 ; CCc1cc(CC)c2oc(COC(=O)Nc3ccn([C@@H]4O[C@H](CO)[C@@H](O)C4(F)F)c(=O)n3)cc2c1 ; O=C(Nc1ccn([C@@H]2O[C@H](CO)[C@@H](O)C2(F)F)c(=O)n1)OCCCCCN1C(=O)C=CC1=O]

22A. A compound of the formula (VI):

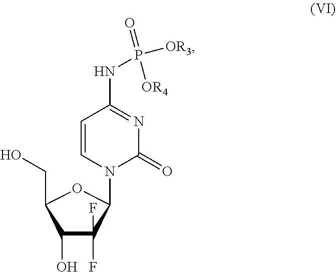

wherein:
   R₃ and R₄ are both H;
   R₃ is H and R₄ is substituted or unsubstituted C₁-C₂₆ alkyl; or,
   R₃ and R₄ are each independently substituted or unsubstituted C₁-C₂₆ alkyl;
or a pharmaceutically acceptable salt thereof.

23A. The compound of clause 22A, wherein one or both of R₃ and R₄ is selected from the group consisting of: —CH₂CH₃, —(CH₂)₂CH₃, —(CH₂)₃CH₃, —(CH₂)₄CH₃, —(CH₂)₅CH₃, —(CH₂)₆CH₃, —CH₂CH(CH₃)₂, —(CH₂)₂CH(CH₃)₂, —(CH₂)₃CH(CH₃)₂, or —(CH₂)₄CH(CH₃)₂.

24A. A compound of formula (IV):

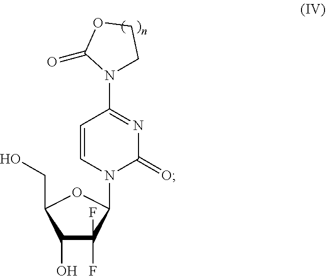

wherein n is 0, 1 or 2;
or a pharmaceutically acceptable salt thereof.

25A. A pharmaceutical composition comprising a compound according to any one of clauses 11A to 24A and a pharmaceutically acceptable carrier.

26A. A compound according to any one of clauses 11A to 24A, or a pharmaceutical composition according to clause 25A, for use in therapy.

27A. A compound according to any one of clauses 11A to 24A, or a pharmaceutical composition according to clause 25A, for use in treating cancer.

28A. The compound or pharmaceutical composition for use according to clause 27A, wherein the cancer is selected from the group consisting of: breast cancer, ovarian cancer, non-small cell lung cancer, pancreatic cancer and bladder cancer.

29A. A method of treating a patient, optionally a human patient, suffering from cancer, the method comprising administering an effective amount of a compound according to any one of clauses 11A to 24A, or a pharmaceutical composition according to clause 25A, to the patient.

30A. The method of clause 29A, wherein the cancer is selected from the group consisting of: breast cancer, ovarian cancer, non-small cell lung cancer, pancreatic cancer and bladder cancer.

Gemcitabine is a first line chemotherapeutic drug that acts against a wide range of solid tumours, such as small cell lung, bladder, pancreatic and breast cancer. It possesses a nucleotide-like structure which “camouflages” the compound enhancing its passage across the cell membrane via nucleoside transporters (NTs). NTs are a group of membrane proteins that transport nucleosides across the cell membrane. Once Gemcitabine enters the cell it undergoes a series of phosphorylations to become active; Gemcitabine is phosphorylated by deoxycytidine kinase (dCK) to produce its monophosphate (dFdCMP) and then phosphorylated again by pyrimidine kinases to its active diphosphate and triphosphate derivatives, dFdCDP and dFdCTP respectively.

One pathway through which Gemcitabine expresses its cytotoxicity is via its diphosphate form (dFdCDF) by inhibiting competitively the integration of deoxycytidine triphosphate (dCTP) into DNA and thus, it impedes the DNA synthesis leading subsequently to cell apoptosis (FIG. 1, pathway B). Another pathway where Gemcitabine expresses its cytotoxicity is through its active form, dFdCDP, which inhibits ribonucleoside diphosphate reductase, an enzyme of DNA synthesis, which permits the formation of nucleoside triphosphates. This results in a significant decrease in cellular dCTP and a change in the ratio of dCTP/dFdCTP in favour of dFdCTP. Alternatively, Gemcitabine inactivation is catalysed by CDA where Gemcitabine is transformed to its inactive metabolite dFdU via the deamination of the 4-(N)-position of Gemcitabine (FIG. 1, pathway A).

FIG. 1. The pathway of Gemcitabine in case of deamination (A) and incorporation to the DNA (B).

In order to overcome the inactivation of Gemcitabine because of its deamination by CDA and increase Gemcitabine cytotoxicity, the present inventors developed an innovative approach. More specifically, the main purpose was to protect 4-(N)-group of Gemcitabine from alkylation or acylation and convert it to a carbamate or a carbonate bond or an amide or a phosphoramidate bond. These prodrug derivatives of Gemcitabine will be hydrolysed under the tumour cell acidic pH conditions and as a result, the native Gemcitabine will be released. Thus, the strategy of the present inventors is to preserve Gemcitabine properties and mitigate the need for high doses because the carbamate bond or an amide or a phosphoramidate bond reduces cytotoxicity for normal cells. In addition, the percentage of dFdU transformation will be reduced. Carbamate bonds and phosphoramidate bonds are pH labile and traceless and also, they are hydrolysed more easily than amide bonds, releasing CO₂.

A series of 4-(N)-Gemcitabine carbamate or phosphate derivatives were developed based on a new synthetic method following a chloroformate strategy. The new synthetic method can occur in one pot; it is rapid and selective to the Gemcitabine 4-(N)-position. The new synthetic method can be performed in a single step without isolation of an intermediate. In addition, it is a quantitative and qualitative method for the synthesis of Gemcitabine prodrugs, low-cost and straightforward while no purification is needed. The one pot synthetic method is of high-yield and also a “green” chemistry reaction with many applications. For example the one pot synthetic method provides access to a number of derivatives which can be further derivatised without the need for protecting other areas of the Gemcitabine molecule.

By the new synthetic method described herein, new 4-(N) substituted Gemcitabine derivatives are provided which may have free 3′- and/or 5′-OH groups or which may have substituted 3′- and/or 5′-OH groups. Typical substituents of 3′- and/or 5′-OH groups are acyl groups, e.g. C₂₋₂₆ acyl groups such as acetyl groups. Free 3′- and/or 5′-OH groups may be converted into substituted groups by known procedures, e.g. by reaction with OH-reactive derivatising agents, e.g. acyl anhydrides or acyl halides or any of substituted or unsubstituted C₁-C₂₆ alkyl, substituted or unsubstituted C₁-C₂₆ haloalkyl, e.g. chloroalkyl, substituted or unsubstituted aryl, substituted or unsubstituted benzyl, substituted or unsubstituted C₂-C₂₆ alkenyl, substituted or unsubstituted C₂-C₂₆ alkynyl, C₁-C₂₆ alkyl substituted with one or more substituted or unsubstituted benzyl groups, C₁-C₂₆ alkyl substituted with one or more substituted or unsubstituted triazole groups or a pharmaceutically acceptable salt thereof.

In certain aspects of the present disclosure, the new 4-(N) substituted Gemcitabine derivatives have a substituent at the 4-(N) position which has a reactive group capable of reacting with the H atom at the 4 (N)-position. For example, the reactive group may be a chloro, bromo or iodo group. By this means, an intramolecular reaction can take place wherein a cyclic substituent at the position 4-(N) is obtained.

In certain aspects of the present disclosure, the new 4-(N) substituted

Gemcitabine derivatives have a substituent at the 4-(N) position which has a reactive group capable of a click reaction with a complementary click-reactive group. For example, the reactive group may be an azido (N₃) group capable of reacting with a complementary alkyne group, a phospine or phosphite (Staudinger ligation), or the reactive group may be an alkyne group capable of reacting with a complementary azido group or a thiol (thiolyne chemistry).

In certain aspects of the present disclosure, the new 4-(N) substituted Gemcitabine derivatives have a substituent at the 4-(N) position which comprises a triazole ring which may have been generated by a click reaction between an alkyne and an azido group.

### BRIEF DESCRIPTION OF THE DRAWINGS

Embodiments of the invention are described below with reference to the accompanying drawings, in which:

FIG. 1. The pathway of Gemcitabine in case of deamination (A) and incorporation to the DNA (B).

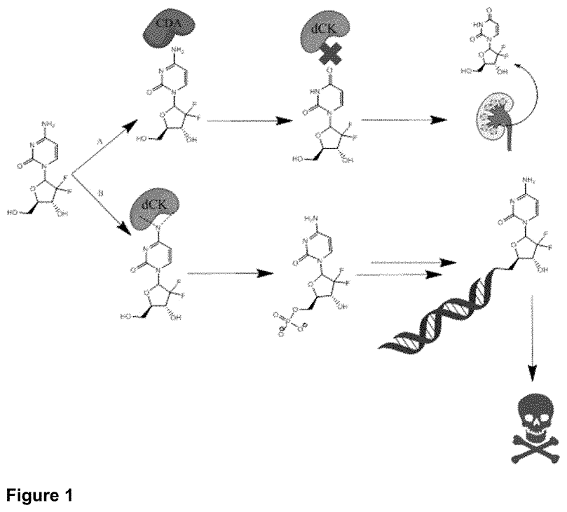

FIG. 2. An application of a photo-cleavable Gemcitabine derivative.

FIG. 3. A possible mechanism of action of phosphorylated Gemcitabine derivatives.

FIG. 4. A. A click reaction between a Gemcitabine derivative bearing an alkyne and coumarin azide. B. The fluorescence spectra of the synthesized compound.

FIG. 5. Time course of the absorption spectrum of derivative 7 during irradiation. Down arrows indicate the peaks belonging to derivative 7, while the arrows pointing up are the peaks created during photolysis.

FIG. 6. Gemcitabine UV spectrum in methanol.

FIG. 7. Results from the confocal microscopy experiments of derivative 11 in HeLa cells.

FIG. 8. Results from the confocal microscopy experiments of derivative 12 in HeLa cells.

FIG. 9. IC₅₀ values of 4-(N)-acyl derivatives in four different cell lines.

FIG. 10. Ratio of IC₅₀ of 4-(N)-acyl derivatives in the presence, compared to in the absence of, dipyridamole, in four cell lines.

FIG. 11. IC₅₀ values of the acetylated 4-(N)-acyl derivatives in four cell lines.

FIG. 12. Ratio of IC₅₀ of the acetylated 4-(N)-acyl derivatives in the presence, compared to in the absence of, dipyridamole, in four cell lines.

FIG. 13. A plot showing cell viability (%) of T-24 cells (5000 cells/well) treated with 100 μM of Gemcitabine derivatives after 24-hour incubation determined by MTT assay.

FIG. 14. A plot showing cell viability (%) of T-24 cells (5000 cells/well) treated with 100 μM of Gemcitabine derivatives after 48-hour incubation determined by MTT assay.

FIG. 15. A plot showing cell viability (%) of T-24 cells (10000 cells/well) treated with 100 μM of Gemcitabine derivatives after 48-hour incubation determined by MTT assay.

FIG. 16. Cytotoxicity of the most efficient Gemcitabine derivatives at different concentrations in the T-24 cell line.

FIG. 17. In vitro stability of Ethyl-(4-N-Gemcitabine) carbamate (derivative 1) after 24h incubation in human plasma at 37° C.

FIG. 18. Calibration curve of Ethyl-(4-N-Gemcitabine) carbamate (derivative 1).

### DETAILED DESCRIPTION OF THE INVENTION

The following description and examples illustrate various embodiments of the present disclosure in detail. Those of skill in the art will recognize that there are numerous variations and modifications of this disclosure that are encompassed by its scope. Accordingly, the description of the disclosed embodiments should not be deemed to limit the scope of the present disclosure.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

“Gemcitabine” refers to the compound 2′,2′-difluoro-2′-deoxycytidine, having the formula I:

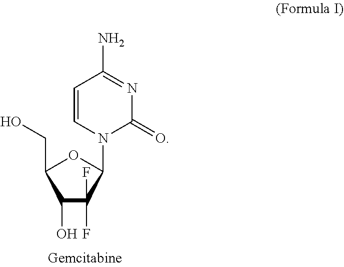

As used herein, any “R” group(s) such as, without limitation, R₁, R₂, R₃, R₄, R₅, R₆, R₇, R₈, R₉, R₁₀, R₁₁, R₁₂, R₁₃, R₁₄, R₂₀, R₂₁, R₂₀₁ and R₂₀₂ represent substituents that can be attached to the indicated atom. An R group may be substituted or unsubstituted. If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

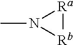

In addition, if two “R” groups are described as being “taken together” with the atom(s) to which they are attached to form a ring as an alternative, the R groups may not be limited to the variables or substituents defined previously.

As used herein, “alkyl” refers to a straight or branched hydrocarbon chain that comprises a fully saturated (no double or triple bonds) hydrocarbon group. The alkyl group may have 1 to 26 carbon atoms (whenever it appears herein, a numerical range such as “1 to 26” refers to each integer in the given range; e.g. “1 to 26 carbon atoms” means that the alkyl group may consist of 1 carbon atom, 2 carbon atoms, 3 carbon atoms, 4 carbon atoms, 5 carbon atom, 6 carbon atoms, 7 carbon atoms, 8 carbon atoms, 9 carbon atom, 10 carbon atoms, 11 carbon atoms, 12 carbon atoms, 13 carbon atoms, 14 carbon atoms, 15 carbon atoms, 16 carbon atoms, 17 carbon atoms, 18 carbon atoms, 19 carbon atoms, 20 carbon atoms, 21 carbon atoms, 22 carbon atoms, 23 carbon atoms, 24 carbon atoms, 25 carbon atoms or 26 carbon atoms, although the present definition also covers the occurrence of the term “alkyl” where no numerical range is designated). The alkyl group may also be a medium size alkyl having from 1 to 10 carbon atoms. The alkyl group could also be a lower alkyl having from 1 to 6 carbon atoms. The alkyl group of the compounds may be designated as “C₁-C₆ alkyl” or similar designations. By way of example only, “C₁-C₆ alkyl” indicates that there are one to six carbon atoms in the alkyl chain, i.e. the alkyl chain is selected from methyl, ethyl, propyl, iso-propyl, n-butyl, iso-butyl, sec-butyl, and t-butyl, pentyl (straight and branched) and hexyl (straight and branched). Typical alkyl groups include, but are in no way limited to, methyl, ethyl, propyl, isopropyl, butyl, isobutyl, tertiary butyl, pentyl (straight and branched) and hexyl (straight and branched). The alkyl group may be mono- or polysubstituted or unsubstituted. Typical substituents can be selected from —OH, —O—C₁₋₆ (optionally halo, e.g. —F, —Cl, —Br or —I)alkyl, —SH, —S—C₁₋₆ alkyl, —N₃, —NO₂, -halo (e.g. —F, —Cl, —Br or —I), —COOH, and/or —COOR₂ (wherein R₂ is substituted or unsubstituted C₁-C₂₆ alkyl).

As used herein, “haloalkyl”, for example “chloroalkyl”, refers to a straight or branched hydrocarbon chain that comprises a fully saturated (no double or triple bonds) hydrocarbon group and at least one halogen atom, for example chlorine atom in the case of “chloroalkyl”, (optionally, one, two, three, four, five or six, or more, halo atoms, for example chlorine atoms). The term “haloalkyl” encompasses fluoroalkyl, chloroalkyl, bromoalkyl and iodoalkyl. The haloalkyl group, for example chloroalkyl group, may have 1 to 26 carbon atoms (whenever it appears herein, a numerical range such as “1 to 26” refers to each integer in the given range; e.g. “1 to 26 carbon atoms” means that the alkyl group may consist of 1 carbon atom, 2 carbon atoms, 3 carbon atoms, 4 carbon atoms, 5 carbon atom, 6 carbon atoms, 7 carbon atoms, 8 carbon atoms, 9 carbon atom, 10 carbon atoms, 11 carbon atoms, 12 carbon atoms, 13 carbon atoms, 14 carbon atoms, 15 carbon atoms, 16 carbon atoms, 17 carbon atoms, 18 carbon atoms, 19 carbon atoms, 20 carbon atoms, 21 carbon atoms, 22 carbon atoms, 23 carbon atoms, 24 carbon atoms, 25 carbon atoms or 26 carbon atoms, although the present definition also covers the occurrence of the term “alkyl” where no numerical range is designated). The chloroalkyl group may also be a medium size chloroalkyl having from 1 to 10 carbon atoms. The chloroalkyl group could also be a lower chloroalkyl having from 1 to 6 carbon atoms. The chloroalkyl group of the compounds may be designated as “C₁-C₆ chloroalkyl” or similar designations. By way of example only, “C₁-C₆ chloroalkyl” indicates that there are one to six carbon atoms in the alkyl chain, i.e. the alkyl chain is selected from, each having at least one chlorine atom, chloromethyl, chloroethyl, chloropropyl, chloro-iso-propyl, chloro-n-butyl, chloro-iso-butyl, chloro-sec-butyl, and chloro-t-butyl, chloropentyl (straight and branched) and chlorohexyl (straight and branched).

Typical chloroalkyl groups include, but are in no way limited to, chloromethyl, chloroethyl, chloropropyl, chloroisopropyl, chlorobutyl, chloroisobutyl, chloro-tertiary butyl, chloropentyl (straight and branched) and chlorohexyl (straight and branched). Analogously, respective fluoroalkyl, bromoalkyl or iodoalkyl groups are included within this definition of haloalkyl. The haloalkyl group, for example chloroalkyl group, may be mono- or polysubstituted or unsubstituted. Typical substituents can be selected from —OH, —O—C₁₋₆ (optionally halo, e.g. —F, —Cl, —Br or —I)alkyl, —SH, —S—C₁₋₆ alkyl, —N₃, —NO₂, -halo (e.g. —F, —Cl, —Br or —I), —COON, and/or —COOR₂ (wherein R₂ is substituted or unsubstituted C₁-C₂₆ alkyl).

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl. Typical substituents can be selected from —OH, —O—C₁₋₆ (optionally halo, e.g. —F, —Cl, —Br or —I)alkyl, —SH, —S—C₁₋₆ alkyl, —N₃, —NO₂, -halo (e.g. —F, —Cl, —Br or —I), —COOH, and/or —COOR₂ (wherein R₂ is substituted or unsubstituted C₁-C₂₆ alkyl).

As used herein, “aryl” refers to a carbocyclic (all carbon) mono-cyclic or multi-cyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be mono- or polysubstituted or unsubstituted. Typical substituents can be selected from —OH, —O—C₁₋₆ (optionally halo, e.g. —F, —Cl, —Br or —I)alkyl, —SH, —S—C₁₋₆ alkyl, —N₃, —NO₂, -halo (e.g. —F, —Cl, —Br or —I), —COOH, and/or —COOR₂ (wherein R₂ is substituted or unsubstituted C₁-C₂₆ alkyl).

As used herein, “alkanoyl” used herein refers to a “carbonyl” substituted with an “alkyl” group, the “alkanoyl” group is covalently bonded to the parent molecule through the carbon of the “carbonyl” group.

As used herein, “cycloalkanoyl” used herein refers to a “carbonyl” substituted with an “cycloalkyl” group, the “alkanoyl” group is covalently bonded to the parent molecule through the carbon of the “carbonyl” group.

As used herein, “alkenoyl” used herein refers to a “carbonyl” substituted with an “alkenyl” group, the “alkenoyl” group is covalently bonded to the parent molecule through the carbon of the “carbonyl” group.

As used herein, “alkynoyl” used herein refers to a “carbonyl” substituted with an “alkynyl” group, the “alkynoyl” group is covalently bonded to the parent molecule through the carbon of the “carbonyl” group.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e. the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl.

Typical alkenyl groups include, but are in no way limited to, ethenyl, propenyl, butenyl, pentenyl, and hexenyl, and the like. An alkenyl group may be mono- or polysubstituted or unsubstituted. Typical substituents can be selected from —OH, —O—C₁₋₆ (optionally halo, e.g. —F, —Cl, —Br or —I)alkyl, —SH, —S—C₁₋₆ alkyl, —N₃, —NO₂, -halo (e.g. —F, —Cl, —Br or —I), —COON, and/or —COOR₂ (wherein R₂ is substituted or unsubstituted C₁-C₂₆ alkyl).

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e. the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like. An alkynyl group may be mono- or polysubstituted or unsubstituted. Typical substituents can be selected from —OH, —O—C₁₋₆ (optionally halo, e.g. —F, —Cl, —Br or —I)alkyl, —SH, —S—C₁₋₆ alkyl, —N₃, —NO₂, -halo (e.g. —F, —Cl, —Br or —I), —COOH, and/or —COOR₂ (wherein R₂ is substituted or unsubstituted C₁-C₂₆ alkyl).

As used herein, “pyranose saccharide” refers to a saccharide having a six-membered ring consisting of five carbon atoms and one oxygen atom. There may be other carbons external to the ring. One non-limiting example of a pyranose saccharide is α-D-glucopyranose:

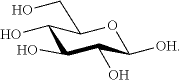

A pyranose saccharide group may be mono- or polysubstituted or unsubstituted. Typical substituents can be selected from —OH, —O—C₁₋₆ (optionally halo, e.g. —F, —Cl, —Br or —I)lkyl, —SH, —S—C₁₋₆ alkyl, —N₃, —NO₂, -halo (e.g. —F, —Cl, —Br or —I), —COOH, and/or —COOR₂ (wherein R₂ is substituted or unsubstituted C₁-C₂₆ alkyl).

As used herein, “furanose saccharide” refers to a saccharide having a five-membered ring consisting of four carbon atoms and one oxygen atom. There may be other carbons external to the ring. One non-limiting example of a furanose saccharide is β-D-fructofuranose: OH

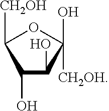

A furanose saccharide group may be mono- or polysubstituted or unsubstituted. Typical substituents can be selected from —OH, —O—C₁₋₆ (optionally halo, e.g. —F, —Cl, —Br or —I)alkyl, —SH, —S—C₁₋₆ alkyl, —N₃, —NO₂, -halo (e.g. —F, —Cl, —Br or —I), —COON, and/or —COOR₂ (wherein R₂ is substituted or unsubstituted C₁-C₂₆ alkyl).

The term “pharmaceutically acceptable salt” refers to a salt of a compound that does not cause significant irritation to an organism to which it is administered and does not abrogate the biological activity and properties of the compound.

In some embodiments, the salt is an acid addition salt of the compound. Pharmaceutical salts can be obtained by reacting a compound with inorganic acids such as hydrohalic acid (e.g. hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e. hydrates) or common organic solvents. Unless otherwise indicated, such solvates are included in the scope of the compounds disclosed herein.

It is to be understood that where compounds disclosed herein have unfilled valencies, then the valencies are to be filled with hydrogens or isotopes thereof, e.g. hydrogen-1 (protium) and hydrogen-2 (deuterium).

It is understood that the compounds described herein can be labelled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound.

At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

As used herein, the term “prodrug” generally refers to a compound, which is pharmaceutically acceptable and upon administration is converted to a desired active compound, here Gemcitabine. In some embodiments, the prodrug can be therapeutically inactive until cleaved to release the active compound. The prodrug will contain an “active” component, in this case Gemcitabine, and a moiety (for example a protecting group) attached to the 4-(N)- position of Gemcitabine. Removal of some or all of the moiety will convert the prodrug from an inactive form to an active drug. This is done in the body by a chemical or biological reaction.

Depending on the moiety (for example a protecting group) attached to the 4-(N)-position of Gemcitabine, the at least one prodrug formed can be either a neutral (uncharged), a free acid, a free base or a pharmaceutically acceptable anionic or cationic salt form or salt mixtures with any ratio between positive and negative components. These anionic salt forms can include, but are not limited to, for example, acetate, 1-aspartate, besylate, bicarbonate, carbonate, d-camsylate, I-camsylate, citrate, edisylate, formate, fumarate, gluconate, hydrobromide/bromide, hydrochloride/chloride, d-lactate, I-lactate, d,I-lactate, d,I-malate, I-malate, mesylate, pamoate, phosphate, succinate, sulfate, bisulfate, d-tartrate, I-tartrate, d,I-tartrate, meso-tartrate, benzoate, gluceptate, d-glucuronate, hybenzate, isethionate, malonate, methylsufate, 2-napsylate, nicotinate, nitrate, orotate, stearate, tosylate, thiocyanate, acefyllinate, aceturate, am inosalicylate, ascorbate, borate, butyrate, camphorate, camphocarbonate, decanoate, hexanoate, cholate, cypionate, dichloroacetate, edentate, ethyl sulfate, furate, fusidate, galactarate (mucate), galacturonate, gallate, gentisate, glutamate, glutamate, glutarate, glycerophosphate, heptanoate (enanthate), hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, or undecylenate. The cationic salt forms can include, but are not limited to, for example, sodium, potassium, calcium, magnesium, zinc, aluminum, lithium, cholinate, lysinium, ammonium, or tromethamine.

The term “pharmaceutically acceptable carriers” includes, but is not limited to, 0.01-0.1 M and preferably 0.05 M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In some embodiments, the carrier can be a) 10% PEG (polyethylene glycol) 400 (v/v) +30% (v/v) HPβCD (hydroxypropyl β-cyclodextrin), 50% w/v+60% (v/v) Sterile Water for Injection or b) 0.1% (v/v) Tween 80+0.5% (w/v) carboxymethylcellulose in water.

The term “subject” refers to a mammal, such as humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, and cats, avian species, such as chickens, turkeys, and songbirds. The subject can be, for example, a child, such as an adolescent, or an adult.

The term “treatment” refers to any treatment of a pathologic condition in a subject, such as a mammal, particularly a human, and includes: (i) preventing and/or reducing the risk of a pathologic condition from occurring in a subject which may be predisposed to the condition but has not yet been diagnosed with the condition and, accordingly, the treatment constitutes prophylactic treatment for the disease condition; (ii) inhibiting and/or reducing the speed of development of the pathologic condition, e.g., arresting its development; (iii) relieving the pathologic condition, e.g., causing regression of the pathologic condition; or (iv) relieving the conditions mediated by the pathologic condition and/or symptoms of the pathologic condition. Treatment of subjects who have previously and/or are currently, and/or are about to receive a cancer therapy are contemplated herein.

The term “therapeutically effective amount” refers to that amount of a compound of the invention that is sufficient to effect treatment, when administered to a subject in need of such treatment. The therapeutically effective amount will vary depending upon the subject and disease condition being treated, the weight and age of the subject, the severity of the disease condition, the manner of administration and the like, which can readily be determined by one of ordinary skill in the art.

Without being limited to the following theory, some of the embodiments of the prodrugs/conjugates provided herein undergo enzyme hydrolysis of the carbamate bond in vivo, which subsequently leads to the provision of

Gemcitabine and the respective, metabolites thereof and/or derivatives and/or components thereof. The blocking moieties, i.e. the moieties attached to Gemcitabine through a carbamate bond, of the present disclosure are non-toxic or have very low toxicity at the given dose levels and are preferably known drugs, natural products, metabolites, or GRAS (Generally Recognized As Safe) compounds (e.g. preservatives, dyes, flavors, etc.) or non-toxic mimetics or derivatives thereof.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

### Compounds

In some embodiments, Gemcitabine derivatives are provided. Particular Gemcitabine derivatives disclosed herein are set out in Table 1 below. The number shown for each Gemcitabine derivative shown in Table 1 is used throughout this specification to refer to the same compound.

Table 1: Gemcitabine derivatives

TABLE 1
Gemcitabine derivatives

| Gemcitabine | |
|---|---|
| Derivative Number | Structure |
| 1 | 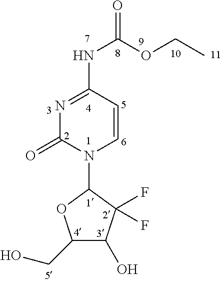 [SMILES: CCOC(=O)Nc1ccn(C2OC(CO)C(O)C2(F)F)c(=O)n1] |
| | Ethyl-(4-N-Gemcitabine) carbamate |
| 2 | 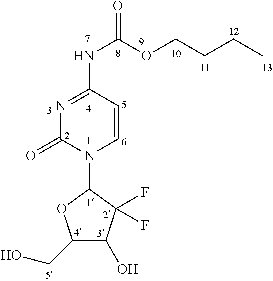 [SMILES: CCCCOC(=O)Nc1ccn(C2OC(CO)C(O)C2(F)F)c(=O)n1] |
| | n-Butyl-(4-N-Gemcitabine) carbamate |
| 3A | 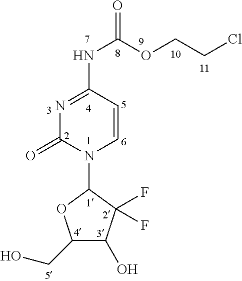 [SMILES: O=C(Nc1ccn(C2OC(CO)C(O)C2(F)F)c(=O)n1)OCCCl] |
| | 2-Chloro-Ethyl-(4-N-Gemcitabine) carbamate |
| 3B | 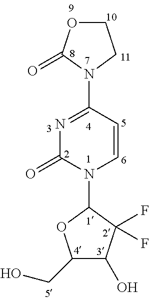 [SMILES: O=C1OCCN1c1ccn(C2OC(CO)C(O)C2(F)F)c(=O)n1] |
| | 2-oxazolidyn-(4-N-Gemcitabine) carbamate |
| 4 | 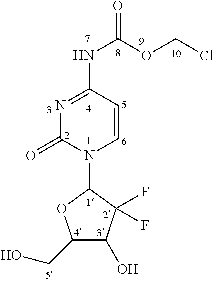 [SMILES: O=C(Nc1ccn(C2OC(CO)C(O)C2(F)F)c(=O)n1)OCCl] |
| | 2-Chloro-Methyl-(4-N-Gemcitabine) carbamate |
| 5 | 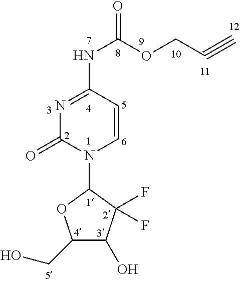 [SMILES: C#CCOC(=O)Nc1ccn(C2OC(CO)C(O)C2(F)F)c(=O)n1] |
| | Propargyl-(4-N-Gemcitabine) carbamate |
| 6 | 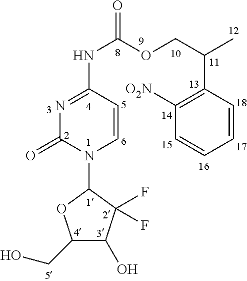 [SMILES: CC(COC(=O)Nc1ccn(C2OC(CO)C(O)C2(F)F)c(=O)n1)c1ccccc1[N+](=O)[O-]] |
| | 2-(2-nitrophenyl) propyl-(4-N- |
| | Gemcitabine) carbamate |
| 7 | 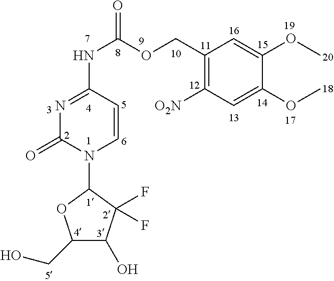 [SMILES: COc1cc(COC(=O)Nc2ccn(C3OC(CO)C(O)C3(F)F)c(=O)n2)c([N+](=O)[O-])cc1OC] |
| | (4,5-dimethoxy)-2-nitrobenzyl-(4-N- |
| | Gemcitabine) carbamate |
| 8 | 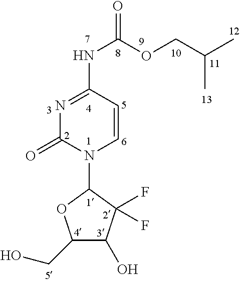 [SMILES: CC(C)COC(=O)Nc1ccn(C2OC(CO)C(O)C2(F)F)c(=O)n1] |
| | (n-isobutyl)-(4-N-Gemcitabine) carbamate |
| 9 | 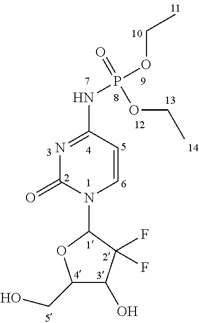 [SMILES: CCOP(=O)(Nc1ccn(C2OC(CO)C(O)C2(F)F)c(=O)n1)OCC] |
| | 4-N-(diethoxy phosphate) Gemcitabine |
| 10 | 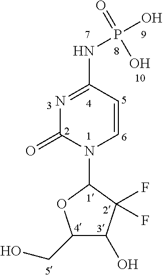 [SMILES: O=c1nc(NP(=O)(O)O)ccn1C1OC(CO)C(O)C1(F)F] |
| | 4-N-(phosphate) Gemcitabine |
| 11 | 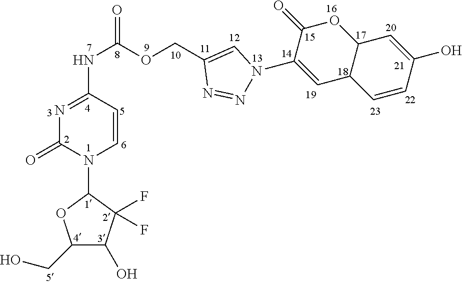 [SMILES: O=C(Nc1ccn(C2OC(CO)C(O)C2(F)F)c(=O)n1)OCc1cn(C2=CC3C=CC(O)=CC3OC2=O)nn1] |
| | (1-(7-hydroxy-2-oxo-4a,8a-dihydro-2H- |
| | chromen-3-yl)-1H-1,2,3-triazol-4-yl) |
| | methyl-4-N-Gemcitabine carbamate |
| 12 | 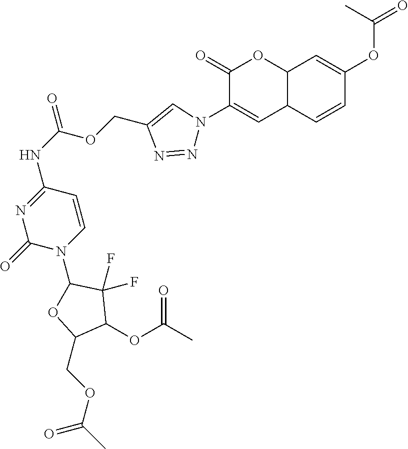 [SMILES: CC(=O)OCC1OC(n2ccc(NC(=O)OCc3cn(C4=CC5C=CC(OC(C)=O)=CC5OC4=O)nn3)nc2=O)C(F)(F)C1OC(C)=O] |
| | 3′,5′-diacetyl (1-(7-acetyl-2-oxo-4a,8a- |
| | dihydro-2H-chromen-3-yl)-1H-1,2,3- |
| | triazol-4-yl) methyl-4-N-Gemcitabine carbamate |
| 13 | 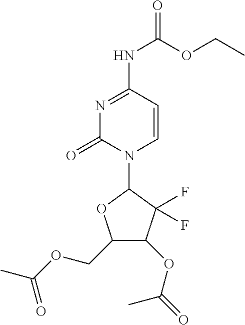 [SMILES: CCOC(=O)Nc1ccn(C2OC(COC(C)=O)C(OC(C)=O)C2(F)F)c(=O)n1] |
| | 3′,5′-diacetyl-(ethyl-4-N-Gemcitabine) carbamate |
| 14 | 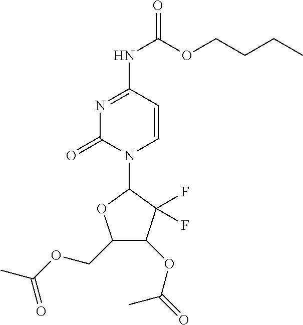 [SMILES: CCCCOC(=O)Nc1ccn(C2OC(COC(C)=O)C(OC(C)=O)C2(F)F)c(=O)n1] |
| | 3′,5′-diacetyl-(n-butyl-4-N-Gemcitabine) carbamate |
| 15 | 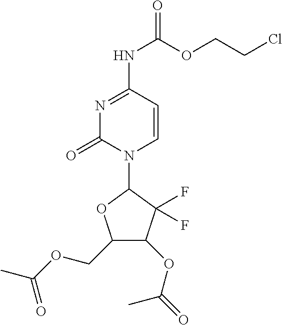 [SMILES: CC(=O)OCC1OC(n2ccc(NC(=O)OCCCl)nc2=O)C(F)(F)C1OC(C)=O] |
| | 3′,5′-diacetyl-(chloroethyl-4-N-Gemcitabine) carbamate |
| 16 | 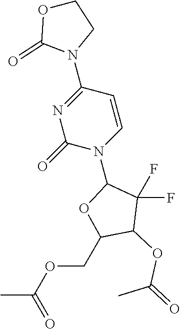 [SMILES: CC(=O)OCC1OC(n2ccc(N3CCOC3=O)nc2=O)C(F)(F)C1OC(C)=O] |
| | 3′,5′-diacetyl-(oxazolidinyl-4-N- |
| | Gemcitabine) carbamate |
| 17 | 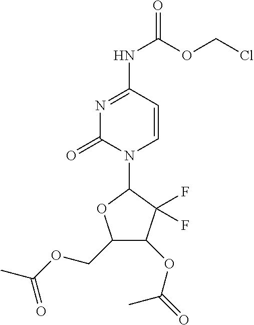 [SMILES: CC(=O)OCC1OC(n2ccc(NC(=O)OCCl)nc2=O)C(F)(F)C1OC(C)=O] |
| | 3′,5′-diacetyl-(chloromethyl-4-N- |
| | Gemcitabine) carbamate |
| 18 | 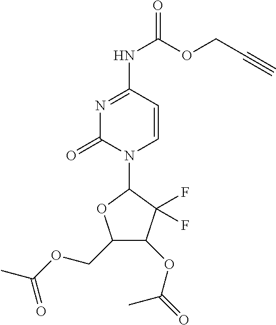 [SMILES: C#CCOC(=O)Nc1ccn(C2OC(COC(C)=O)C(OC(C)=O)C2(F)F)c(=O)n1] |
| | 3′,5′-diacetyl-(propargyl-4-N- |
| | Gemcitabine) carbamate |
| 19 | 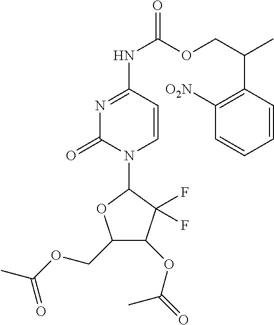 [SMILES: CC(=O)OCC1OC(n2ccc(NC(=O)OCC(C)c3ccccc3[N+](=O)[O-])nc2=O)C(F)(F)C1OC(C)=O] |
| | 3′,5′-diacetyl-(2-(2-nitrophenyl) propyl- |
| | 4-N-Gemcitabine) carbamate |
| 20 | 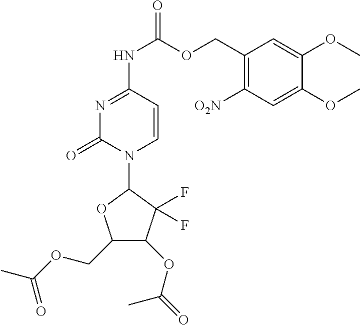 [SMILES: COc1cc(COC(=O)Nc2ccn(C3OC(COC(C)=O)C(OC(C)=O)C3(F)F)c(=O)n2)c([N+](=O)[O-])cc1OC] |
| | 3′,5′-diacetyl-((4,5-dimethoxy)-2- |
| | nitrobenzyl-4-N-Gemcitabine)) carbamate |
| 21 | 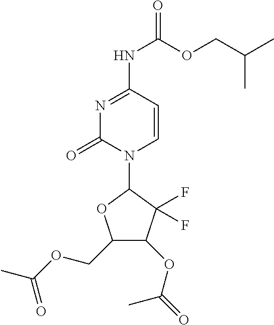 [SMILES: CC(=O)OCC1OC(n2ccc(NC(=O)OCC(C)C)nc2=O)C(F)(F)C1OC(C)=O] |
| | 3′,5′-diacetyl-(n-isobutyl-4-N- |
| | Gemcitabine) carbamate |
| 22 | 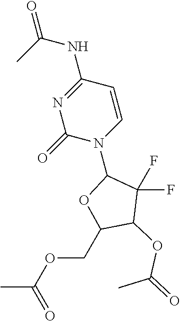 [SMILES: CC(=O)Nc1ccn(C2OC(COC(C)=O)C(OC(C)=O)C2(F)F)c(=O)n1] |
| | 3′,5′,4-N-triacetyl Gemcitabine |

### Synthetic Methods

A. General synthetic procedure for the production of Gemcitabine derivatives 1 to 11: Under a nitrogen atmosphere, Gemcitabine (30 mg, 0.114 mmol) was mixed with 15 ml ethyl acetate/acetonitrile solution (2:1, v/v) under reflux for 1 h (Observation: Gemcitabine becomes soluble and the reaction mixture turns almost clear). Ethylchloroformate (when forming derivative 1) (5.440 μl, 0.057 mmol) was added in the mixture and reflux continued. Reaction progress was monitored with TLC (DCM/acetone/ethanol, 5/5/0.5, v/v). After 2 h, the reaction mixture was centrifuged and the mother liquor concentrated and dried under high vacuum, giving 14.6 mg (98.31%) white solid. (A similar reaction was attempted using a primary alkylbromide rather than ethylchloroformate—the reaction was unsuccessful). The range of amounts of ethylchloroformate present in the reaction was optionally from 0.3 to 0.7 equivalents (by moles); optionally, 0.5 equivalents (by moles). Any more than 0.7 moles and the 3′-and/or 5′-OH groups of Gemcitabine were partially protected; any less than 0.3 moles and the 4-(N)-position of Gemcitabine was not adequately protected).

For the other compounds 1 to 11, the same general procedure was followed (in the same molar amounts for the corresponding acyl chloride or phosphoryl chloride) with the additional conditions set out in Table 2 below. Compound 10 can be obtained from compound 9 after cleavage of the ethyl groups with Trimethylsilyl iodide (TMSI)

### Table 2: Formation of Gemcitabine Derivatives 1 to 11:

TABLE 2
Formation of Gemcitabine derivatives 1 to 11:

| Formation of | Chloroformate starting material to be | Reaction | | |
|---|---|---|---|---|
| derivative | installed in the 4-N of Gemcitabine | time (h) | Yield/% | |
| 1 | 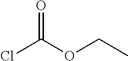 | 3 | 98.3 | |
| 2 | 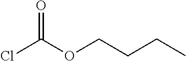 | 3 | 98.2 | |
| 3A | 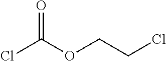 | 3 12 48 | 76.4 84.9 84.9 | 3A = 100% 3A = 74.55% 3B = 25.45% 3A = 8% |
| | | | | 3B = 92% |
| 3B | 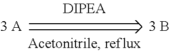 | 2 h | 100 | 3B = 100% |
| 4 | 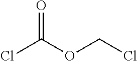 | 3 | 91.4 | |
| 5 | 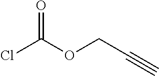 | 3 | 97.7 | |
| 6 | 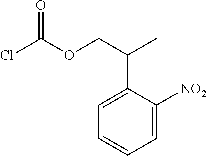 [SMILES: CC(COC(=O)Cl)c1ccccc1[N+](=O)[O-]] | 3 | 95.8 | |
| 7 | 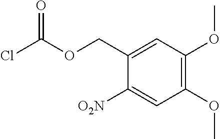 [SMILES: COc1cc(COC(=O)Cl)c([N+](=O)[O-])cc1OC] | 3 | 71.2 | |
| 8 | 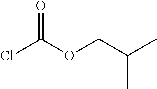 | 3 | 88.1 | |
| 9 | 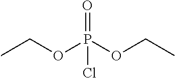 | 8 | 66.5 | |
| 10 | 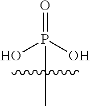 [SMILES: *P(=O)(O)O] | 4 | 84.2% | |
| | Compound 10 is obtained from | | | |
| | compound 9 after cleavage of the ethyl | | | |
| | groups with Trimethylsilyl iodide (TMSI) | | | |
| 11 | 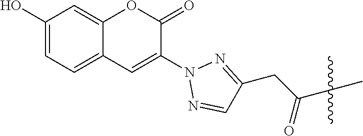 [SMILES: *C(=O)Cc1cnn(-c2cc3ccc(O)cc3oc2=O)n1] | 8 | 70.5% | |
| | Compound 11 is obtained from | | | |
| | compound 5 after click reaction with 7- | | | |
| | hydroxy-3-azido-coumarin | | | |

B. General procedure for click reaction (to form derivative 11): propargyl-(4-N-Gemcitabine) carbamate (10 mg, 0.02898 mmol), Coumarin azide (5.88 mg, 0.02898mmol), triethylamine 10%, Cul 1% and THPTA (0.1%) (catalyst) were dissolved in 1 ml solution of methanol/H₂O (2:1 v/v), overnight at room temperature. TLC analysis of the final product took place in acetone/DCM (dichloromethane) 1:1 v/v and the results showed that all of the starting materials were consumed and the formation of new spot (fluorescent active at 365 nm took place). The reaction solvent was evaporated to dryness with the use of rotary evaporator and the residue was washed with diethyl ether solution several times. Based on the TLC analysis, the impurities were removed in diethyl ether solution and the final product was a brown solid (70.5% yield).
C. General procedure for acetylation for the production of Gemcitabine derivatives 12 to 22: Under nitrogen atmosphere to a solution of our starting chloroformate (1 equivalent) in pyridine (2 ml) and DMAP (4-(dimethylamino)-pyridine; catalytic quantity), acetic anhydride (n equivalents, n=number of —OH groups) was added and the reaction was stirred for 3h at room temperature. The clear solution was concentrated with distillation. The crude mixture was dissolved in EtOAc and washed with saturated NaHCO₃ and brine. The organic layer was dried over Na₂SO₄, filtered and concentrated under reduced pressure to give the desired compound. The reaction was monitored using TLC acetone/DCM 1:1 v/v and shows transformation of the starting material.

For the Gemcitabine derivatives 12 to 22, the same general procedure was followed with the additional conditions set out in Table 3 below

### Table 3: Formation of Gemcitabine Derivatives 12 to 22:

TABLE 3
Formation of Gemcitabine derivatives 12 to 22:

| Compound | Acetylated Chloroformates | Reaction time | Purification | Yield |
|---|---|---|---|---|
| 12 | 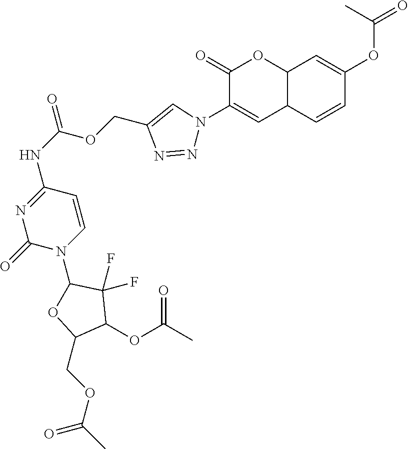 [SMILES: CC(=O)OCC1OC(n2ccc(NC(=O)OCc3cn(C4=CC5C=CC(OC(C)=O)=CC5OC4=O)nn3)nc2=O)C(F)(F)C1OC(C)=O] | 3 h Reflux 120° C. | HPLC | 78.3% |
| 13 | 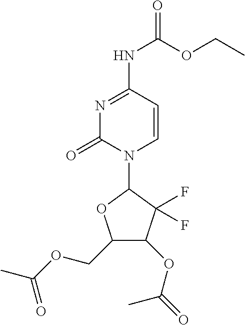 [SMILES: CCOC(=O)Nc1ccn(C2OC(COC(C)=O)C(OC(C)=O)C2(F)F)c(=O)n1] | 3 h Room temperature | Extraction with saturated solution of (CuSO₄), saturated solution of (NaHCO₃) and Brine. | 86.3% |
| 14 | 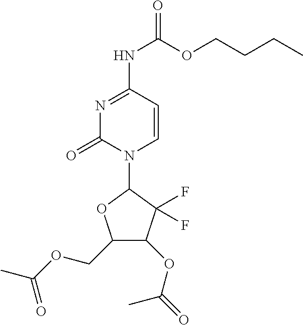 [SMILES: CCCCOC(=O)Nc1ccn(C2OC(COC(C)=O)C(OC(C)=O)C2(F)F)c(=O)n1] | 3 h Room temperature | Extraction with saturated solution of (CuSO₄), saturated solution of (NaHCO₃) and Brine. | 90% |
| 15 | 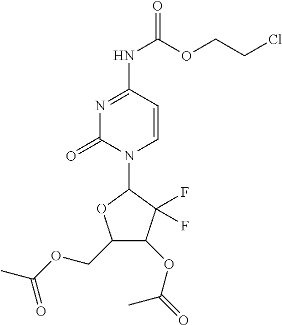 [SMILES: CC(=O)OCC1OC(n2ccc(NC(=O)OCCCl)nc2=O)C(F)(F)C1OC(C)=O] | 2 h Room temperature (Extended reaction time leads to the cyclized byproduct) | Extraction with saturated solution of (CuSO₄), saturated solution of (NaHCO₃), brine and then subjected to HPLC. | 65.8% |
| 16 | 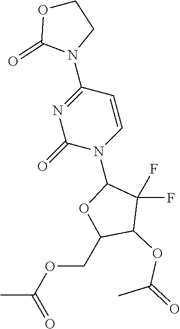 [SMILES: CC(=O)OCC1OC(n2ccc(N3CCOC3=O)nc2=O)C(F)(F)C1OC(C)=O] | 3 h Room temperature | Extraction with saturated solution of (CuSO₄), saturated solution of (NaHCO₃) and Brine. | 81.9% |
| 17 | 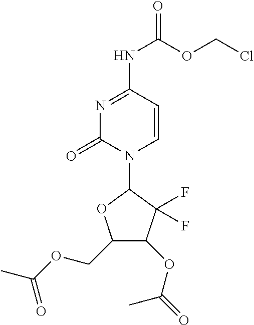 [SMILES: CC(=O)OCC1OC(n2ccc(NC(=O)OCCl)nc2=O)C(F)(F)C1OC(C)=O] | 1 h Room temperature (Extended reaction time leads to the triacetylated derivative.) | Extraction with saturated solution of (CuSO₄), saturated solution of (NaHCO₃), brine and then subjected to HPLC. | 46% |
| 18 | 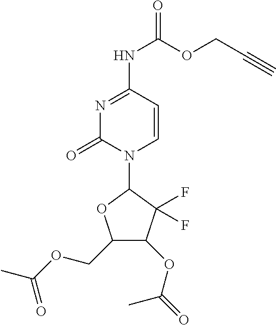 [SMILES: C#CCOC(=O)Nc1ccn(C2OC(COC(C)=O)C(OC(C)=O)C2(F)F)c(=O)n1] | 3 h Room temperature | Extraction with saturated solution of (CuSO₄), saturated solution of (NaHCO₃) and Brine. | 88.5% |
| 19 | 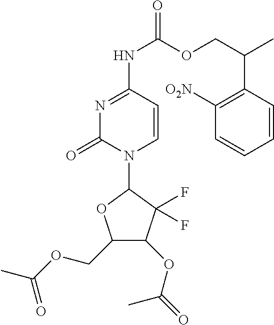 [SMILES: CC(=O)OCC1OC(n2ccc(NC(=O)OCC(C)c3ccccc3[N+](=O)[O-])nc2=O)C(F)(F)C1OC(C)=O] | 3 h Room temperature | Extraction with saturated solution of (CuSO₄), saturated solution of (NaHCO₃) and Brine. | 79.1% |
| 20 | 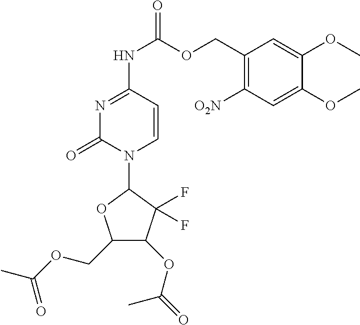 [SMILES: COc1cc(COC(=O)Nc2ccn(C3OC(COC(C)=O)C(OC(C)=O)C3(F)F)c(=O)n2)c([N+](=O)[O-])cc1OC] | 3 h Room temperature | Extraction with saturated solution of (CuSO₄), saturated solution of (NaHCO₃) and Brine (NaCl). | 70.4% |
| 21 | 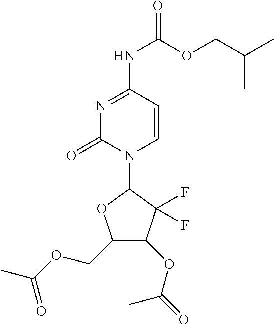 [SMILES: CC(=O)OCC1OC(n2ccc(NC(=O)OCC(C)C)nc2=O)C(F)(F)C1OC(C)=O] | 3 h Room temperature | Extraction with saturated solution of (CuSO₄), saturated solution of (NaHCO₃) and Brine. | 87% |
| 22 | 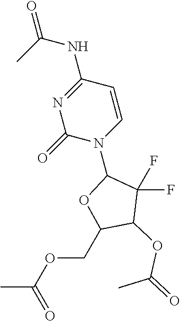 [SMILES: CC(=O)Nc1ccn(C2OC(COC(C)=O)C(OC(C)=O)C2(F)F)c(=O)n1] | 2 h Room temperature | Extraction with saturated solution of (CuSO₄), saturated solution of (NaHCO₃) and Brine. | 93.3% |

### Intermediates

Whilst the biological data in the present application shows the biological activities of many of these compounds, some of the compounds also act as useful intermediates in the formation of further derivatised versions of Gemcitabine, for example conjugates of Gemcitabine. The presently disclosed one pot synthesis of 4-(N)-protected-Gemcitabine derivatives provides a synthetic route to 4-(N)-Gemcitabine-conjugate compounds.

### Photocaged Gemcitabine

Photochemistry provides a mechanism for actively controlling the release of a drug selectively to the cancer site for the purpose of targeted drug delivery. Compound 7 can be detected from the irradiation release of Gemcitabine, with the use of a photodegradable linking strategy. The term opaque refers to the temporary inactivation of a biologically active molecule using a protective photodegradable group. As photodegradable linking group, the present inventors used an ortho-nitrobenzyl (CNB) group with selective modification to the primary amine of Gemcitabine.

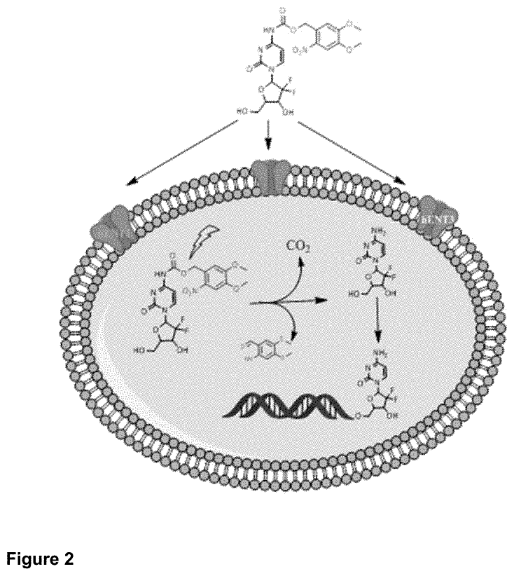

After ultraviolet irradiation at a specific photo-digested group wavelength in the range from 350 to 500 nm (or greater than 700 nm when utilising two photon excitation), the active form of the encapsulated molecule is released irreversibly. Photo-inclusion has often been performed in vitro for the spatio-temporal control of biological processes and the release of light-induced payload. This dual in drug release approach (cell targeting and photo-controlled release) could be more effective in enhancing the therapeutic index of an anticancer drug than either mechanisms alone. The present inventors believe that this dual strategy is of great value for therapeutic applications while it requires non-invasive and space-time drug activation. In order to show this dual drug release mechanism, the present inventors performed photolysis experiments (FIG. 2).

FIG. 2. An application of a photo-cleavable Gemcitabine derivative (derivative 7).

Enzyme activation and drug release.

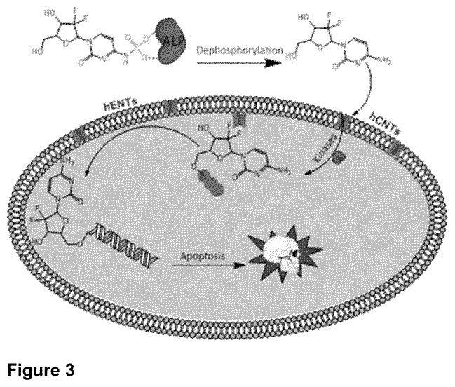

Another mechanism where selective release of the active drug can occur is under the action of certain enzymes overexpressed in cancer cells and detected either intracellularly or extracellularly. The design of the prodrugs is based on the fact that these enzymes recognize specific substrates. A representative class of these enzymes is Alkaline Phosphatase (ALP). ALP is a member of the metalloproteinase family, which catalyzes phosphoric ester hydrolysis reactions. Elevated levels of ALP have been directly linked to the appearance of various forms of cancer, especially breast cancer. Based on the action mechanism of ALP, several prodrugs have been designed which exhibit increased water solubility when they are released into cancer cells compared to their parent compounds (FIG. 3).

FIG. 3. A possible mechanism of action of phosphorylated Gemcitabine derivatives (for example derivatives 9 and 10).

The mechanism illustrated in FIG. 3 is drawn on the phosphate derivatives because the phosphate group (in the phosphate derivatives) is recognized by the alkaline phosphatase. The present inventors can utilise the same mechanism to install different chemotypes as stimulus to other enzymes, including but not limited to nitroreductase and β-galactosidase. Besides enzymes, other molecules can be utilised as triggers, including but not limited to glutathione or H₂O₂, using a thiol ether or ester group or a boron ester, respectively.

Cellular Imaging and Localization

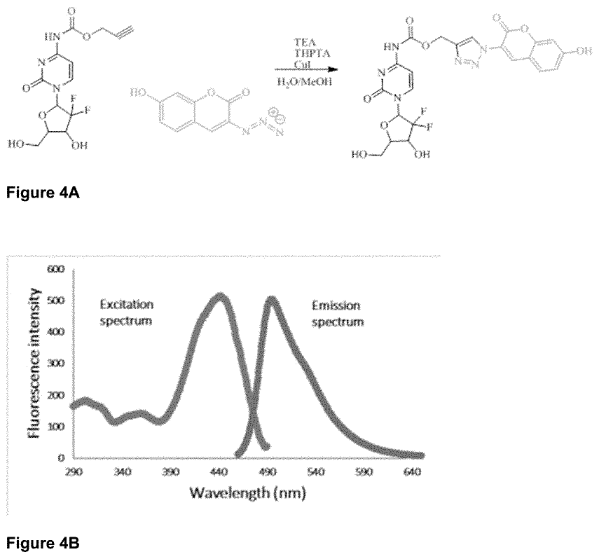

The development of Gemcitabine prodrugs using chloroformate esters inspired the present inventors to construct a molecule for in vivo monitoring of Gemcitabine while it is equipped with the fluorophore agent, coumarin. Conjugation took place with a click chemistry reaction, between an alkyne (derivative 5 in table 1) and an azide (7-hydroxy-3-azido coumarin) to produce compound 11 (FIG. 4).

FIG. 4. A. A click reaction between a Gemcitabine derivative bearing an alkyne and coumarin azide. B. The fluorescence spectra of the synthesized compound (derivative 11).

The use of derivative 11, and related derivatives, either on its own or in combination with Gemcitabine and/or other Gemcitabine derivatives provides a compound with a particular fluorescence spectrum (FIG. 4B). This spectrum can be monitored in vivo or in vitro to test the presence and or action of Gemcitabine and Gemcitabine prodrugs. This is particularly useful in in vitro tests. The in vivo application can be less useful due to low emission wavelength of coumarin as a dye. For in vitro applications, dyes emitting in the near infrared are preferred because they can be utilized for in vivo imaging due to the deep tissue penetration of near infrared.

Characterisation Data

The following characterisation data was obtained for the Gemcitabine derivatives shown in Table 1, which were produced following the synthetic methods described above.

Nuclear magnetic resonance (NMR) spectra were recorded on a Bruker AV500, AV400 and AV250 NMR spectrometer (Bruker, Germany) in deuterated dimethyl sulfoxide (DMSO-d₆) solution and the chemical shifts were determined relative to the residual solvent peak (OH2.50 for DMSO). The following abbreviations are employed to indicate signal multiplicity: s, singlet; d, doublet; t, triplet; q, quartet; m, multiplet; dd, doublet of doublets. Electrospray ionization mass spectrometry (ESI-MS) was conducted on a an Agilent 1100 Series LC/MSD instrument and a EVOQ Elite ER triple quadrupole mass spectrometer (Bruker Daltonics, Germany).

Derivative 1

¹H-NMR of derivative 1: (500 MHz, DMSO-d₆, 25° C.): δ=10.86 (s, 1 H, 7 NH), 8.25 (d, J=7.5 Hz, 1 H, 6-H), 7.14 (d, J=7.5 Hz, 1 H, 5-H), 6.34 (d, J=6.5 Hz, 1 H, 3′-OH), 6.19 (t, J=7.5 Hz, 1 H, 1′-H), 5.32 (t, J=4.5 Hz, 1 H, 5′-OH), 4.22 (m, 1 H, 3′-H), 4.20 (q, J=7.1 Hz, 7.0 Hz, 2 H, 10-H), 2.92 (m,1 H, 4′-H), 2.83 (d, J=12.3 Hz, 1 H, 5′a-H), 2.68 (m, 1 H, 5′b-H) 1.26 (t, J=7.0Hz, 3 H, 11-H) ppm.

¹³C-NMR of derivative 1:(500 MHz, DMSO-d₆, 25° C.): δ=164.3 (C4), 154.99 (C2), 154.04 (C8), 145.19 (C6), 122.86 (C2′), 95.63 (C5), 84.86 (C1′), 81.73 (C4′), 69.14 (C3′), 62.22 (C10), 59.6 (C5′), 15.01 (C11) ppm.

MS (ESI+) m/z: [M+H]+ for C₁₂H₁₅F₂N₃O₆:calcd, 335.09; found, 336.51, [M+Na]+ for C₁₂H₁₅F₂N₃O₆Na: calcd, 357.49; found, 358.49, [M+K]+ for C₁₂H₁₅F₂N₃O₆K: calcd, 373.09; found, 374.51.

Derivative 2

¹H-NMR of derivative 2: (500 MHz, DMSO-d₆, 25° C.): δ=10.85 (s, 1 H, 7-NH), 8.25 (d, J=7.65 Hz, 1 H, 6-H), 7.13 (d, J=7.65 Hz, 1 H, 5-H), 6.34 (d, J =6.50 Hz, 1 H, 3′-OH), 6.19 (t, J=7.50 Hz, 1 H, 1′-H), 5.32 (t, J=5.50 Hz, 1 H, 5′-OH), 4.22 (m, 1 H, 3′-H), 4.15 (t, J=6.60 Hz, 2 H, 10-H), 2.91 (m, 1 H, 4′-H), 2.83 (m, 1 H, 5′a-H), 2.68 (m, 1 H, 5′b-H) 1.62 (m, 2 H, 11-H), 1.39 (m, 2H, 12-H), 0.94 (t, J=7.35 Hz, 3 H, 13-H) ppm.

¹³C-NMR of derivative 2: (500 MHz, DMSO-d₆, 25° C.): δ=164.38 (C4), 154.88 (C2), 154.18 (C8), 145.33 (C6), 122.9 (C2′), 95.55 (C5), 84.76 (C1′), 81.65 (C4′), 69.09 (C3′), 65.75 (C10), 59.48 (C5′), 30.94 (C11), 19.14 (C12), 14.16 (C13) ppm.

Derivative 3A

¹H-NMR of derivative 3A: (500 MHz, DMSO-d₆, 25° C.): δ=11.03 (s, 1 H, 7-NH), 8.27 (d, J=7.70 Hz, 1 H, 6-H), 7.11 (d, J=7.70 Hz, 1 H, 5-H), 6.20 (t, J =7.60 Hz, 1 H, 1′-H), 4.43 (t, J=5.30 Hz, 2 H, 10-H), 4.22 (m, 1 H, 3′-H), 2.92 (m, 1 H, 4′-H), 2.89 (t, J=5.30 Hz, 2 H, 11-H), 2.84 (m, 1 H, 5′a-H), 2.69 (m, 1 H, 5′b-H) ppm.

¹³C-NMR of derivative 3A: (500 MHz, DMSO-d₆, 25° C.): δ=164.3 (C4), 154.82 (C2), 152.68 (C8), 145.5 (C6), 129.89 (C2′), 95.64 (C5), 84.79 (C1′), 81.68 (C4′), 69.03 (C3′), 65.93 (C10), 59.45 (C5′), 42.16 (C11) ppm.

Derivative 38

¹H-NMR of derivative 3B: (500 MHz, DMSO-d₆, 25° C.): δ=8.29 (d, J=7.70 Hz, 1 H, 6-H), 7.36 (d, J=7.70 Hz, 1 H, 5-H), 6.22 (t, J=7.60 Hz, 1 H, 1′-H), 4.48 (t, J=8.0 Hz, 2 H, 10-H), 4.22 (m, 1 H, 3′-H), 4.10 (m, 2 H, 11-H), 2.92 (m, 1 H, 4′-H), 2.84 (m, 1 H, 5′a-H), 2.69 (m, 1 H, 5′bH) ppm.

¹³C-NMR of derivative 3B: (500 MHz, DMSO-d₆, 25° C.): δ=162.0 (C4), 155.03 (C8), 154.82 (C2), 144.45 (C6), 122.89 (C2′), 95.64 (C5), 84.79 (C1′), 81.68 (C4′), 69.03 (C3′), 62.37 (C10), 59.45 (C5′), 42.20 (C11) ppm.

Derivative 4

¹H-NMR of derivative 4: (500 MHz, DMSO-d₆, 25° C.): δ=11.37 (s, 1 H, 7 NH), 8.28 (d, J=7.6 Hz, 1 H, 6-H), 7.06 (d, J=7.5 Hz, 1 H, 5-H), 6.34 (s broad, 1 H, 3′-OH), 6.16 (t, J=7.4 Hz, 1 H, 1′-H), 5.94 (s, 2H, 10-H), 5.32 (broad, 1 H, 5′-OH), 4.19 (m, 1H, 3′-H), 2.89 (m,1 H, 4′-H), 2.81 (d, J=12.3 Hz, 1 H, 5′a-H), 2.64 (m, 1 H, 5′b-H) ppm.

¹³C-NMR of derivative 4: (500 MHz, DMSO-d₆, 25° C.): δ=164.3 (C4), 154.82 (C2), 152.68 (C8), 146.1 (C6), 129.89 (C2′), 96 (C5), 84.8 (C1′),71.8 (C10), 68.9 (C3′), 81,9 (C4′), 59,6 (C5′) ppm.

Derivative 5

¹H-NMR of derivative 5: (500 MHz, DMSO-d₆, 25° C.): δ=11.10 (s, 1 H, 7-NH), 8.28 (d, J=7.6 Hz, 1 H, 6-H), 7.11 (d, J=7.6 Hz, 1 H, 5-H), 6.20 (t, J=7.4 Hz, 1 H, 1′-H), 4.83 (d, J=2.35 Hz, 2 H, 10-H), 4.22 (m, 1 H, 3′-H), 2.92 (m, 1 H, 4′-H), 2.84 (m, 1 H, 5′a-H), 2.69 (m, 1 H, 5′bH) 2.65 (t, J=2.35 Hz, 1 H, 12-H) ppm.

¹³C-NMR of derivative 5: (500 MHz, DMSO-d₆, 25° C.): δ=164.20 (C4), 154.81 (C2), 152.31 (C8), 145.95 (C6), 122.93 (C2′), 95.53 (C5), 84.83 (C1′), 81.68 (C4′), 79.17 (C11), 78.91 (C12), 69.05 (C3′), 59.45 (C5′) ppm.

Derivative 6

¹H-NMR of derivative 6: (500 MHz, DMSO-d₆, 25° C.): δ=10.89 (s, 1 H, 7-NH), 8.23 (d, J=7.65 Hz, 1 H, 6-H), 7.87 (d, J=7.75 Hz, 1 H, 15-H), 7.81 (d, J=7.75 Hz, 1 H, 18-H), 7.72 (t, J=7.75 Hz, 1 H, 17-H), 7.52 (t, J=7.75 Hz, 1 H, 16-H), 7.03 (m, 1 H, 5-H), 6.19 (t, J=7.50 Hz, 1 H, 1′-H), 4.36 (m, 2 H, 10-H), 4.22 (m, 1 H, 3′-H), 2.91 (m, 1 H, 4′-H), 2.83 (m, 1 H, 5′a-H), 2.68 (m, 1 H, 5′b-H) 2.54 (m, 1 H, 11-), 1.35 (d, J=7.0 Hz, 3 H, 12-H) ppm.

¹³C-NMR of derivative 6: (500 MHz, DMSO-d₆, 25° C.): δ=164.17 (C4), 154.78 (C2), 152.86 (C8), 150 (C18), 145.33 (C6), 137.31 (C13), 132.71 (C15), 129.75 (C17), 128.61 (C16), 124.46 (C14), 122.74 (C2′), 95.53 (C5), 84.80 (C1′), 81.68 (C4′), 69.47 (C10), 69.05 (C3′), 59.47 (C5′), 32.61 (C11), 18.49 (C12) ppm.

Derivative 7

¹H-NMR of derivative 7: (500 MHz, DMSO-d₆, 25° C.): δ=11.30 (s, 1 H, 7-NH), 8.30 (d, J=7.55 Hz, 1 H, 6-H), 7.78 (s, 1 H, 13-H), 7.44 (s, 1 H, 16-H), 7.17 (d, J=7.55 Hz, 1 H, 5-H), 6.35 (d, J=6.32 Hz, 1 H, 3′-OH), 6.21 (t, J=7.50 Hz, 1 H, 1′-H), 5.55 (s, 2 H, 10-H), 5.33 (t, J=5 Hz, 1 H, 5′-OH), 4.23 (m, 1 H, 3′-H), 2.99 (s, 3 H, 18-H), 2.93 (m, 1 H, 4′-H), 2.92 (s, 3 H, 17-H), 2.84 (m, 1 H, 5′a-H), 2.69 (m, 1 H, 5′b-H) ppm.

¹³C-NMR of derivative 7: (500 MHz, DMSO-d₆, 25° C.): δ=164.26 (C4), 154.8 (C2), 154.52 (C15), 152.42 (C8), 148.64 (C14), 145.51 (C6), 139.84 (C12), 127.52 (C11), 122.9 (C2′), 111.04 (C16), 108.78 (C13), 95.42 (C5), 84.77 (C1′), 81.68 (C4′), 69.06 (C3′), 64.55 (C10), 59.45 (C5′), 57.18 (C18), 56.75 (C17) ppm.

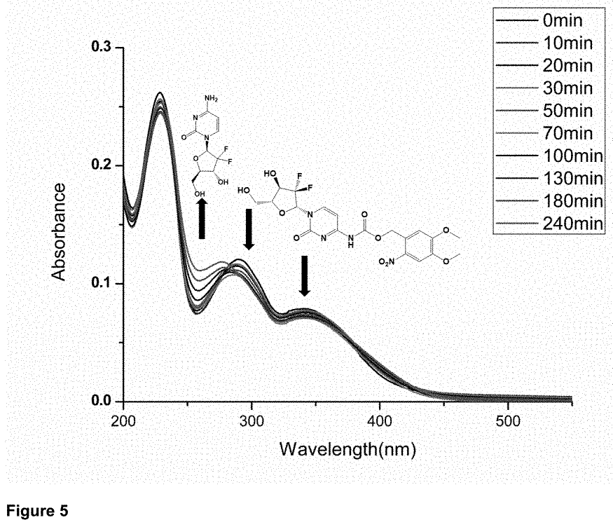

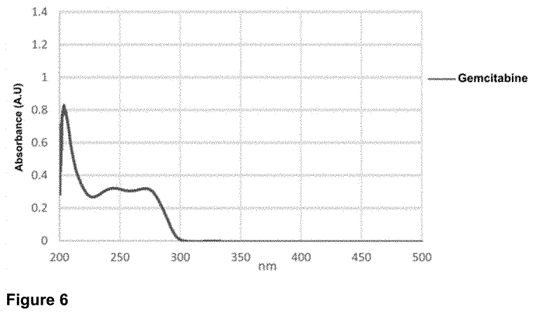

The spectra of derivative 7 (10 μM) and Gemcitabine (5 μM) were recorded on a Perkin Elmer Lambda 25 spectrometer at room temperature. The samples were solubilized in MeOH grade HPLC grade and irradiated with UV lamp at 366 nm for 240 minutes. The results are shown in FIG. 5. The present inventors noticed that in the range of 280-290 nm and 320-330 nm there is a decrease in the absorption intensity and at the same time there is an increase in intensity to 250-270 nm. Without wishing to be bound by theory, the increase in this area is observed due to the release of Gemcitabine. This theory is supported by the increase in the band corresponding to Gemcitabine. As a comparison, the UV spectrum of Gemcitabine is shown in FIG. 6.

FIG. 6. Gemcitabine UV spectrum in methanol.

Derivative 8

1H-NMR of derivative 8: (500 MHz, DMSO-d₆, 25° C.): δ=10.84 (s, 1 H, 7-NH), 8.20 (d, J=7.65 Hz, 1 H, 6-H), 7.08 (d, J=7.65 Hz, 1 H, 5-H), 6.29 (d, J =6.50 Hz, 1 H, 3′-OH), 6.16 (t, J=7.54 Hz, 1 H, 1′-H), 5.27 (t, J=5.32 Hz, 1 H, 5′-OH), 4.18 (m, 1 H, 3′-H), 2.90 (d, J=6.64 Hz, 2 H, 10-H), 2.88 (m, 1 H, 4′-H), 2.80 (m, 1 H, 5′a-H), 2.65 (m, 1 H, 5′b-H) 1.90 (m, 1 H, 11-H), 0.90 (d, J =6.68 Hz, 6 H, 12,13-H) ppm.

Derivative 9

¹H-NMR of derivative 9: (500 MHz, DMSO-d₆, 25° C.): δ=δ=11.84 (s, 1 H, 7-NH), 8.25 (d, J=7.68 Hz, 1 H, 6-H), 7.33 (d, J=7.68 Hz, 1 H, 5-H), 6.32 (d, J =6.52 Hz, 1 H, 3′-OH), 6.18 (t, J=7.46 Hz, 1 H, 1′-H), 5.30 (t, J=5.40 Hz, 1 H, 5′-OH), 4.19 (m, 1 H, 3′-H), 4.06 (m, 4H, 10,13-H), 2.90 (m, 1 H, 4′-H), 2.81 (m, 1 H, 5′a-H), 2.66 (m, 1 H, 5′b-H), 1.24 (t, J=12.91 Hz, 6H, 11,14-H)

Mass of derivative 9: MS (ES1+) m/z: [M+H⁺] for C₁₃H₂₀F₂N₃O₇P: calculated, 399.1 found, 400, [M+N⁺] for C₁₃H₂₀F₂N₃O₇PNa: calculated, 422.08; found, 422.1, [M+K⁺] for C₁₃H₂₀F₂N₃O₇PK: calculated, 438.06 found, 437.9.

Derivative 10 ¹H-NMR of derivative 10: (500 MHz, DMSO-d6, 25° C): δ=8.75 (s, 1 H, 7-NH), 8.13 (s, 2 H, 9,10-H), 7.94 (d, J=7.63 Hz, 1 H, 5-H), 6.34 (broad, 1 H, 3′-OH), 6.10 (t, J=15,19 Hz, 1 H, 1′-H), 5.99 (d, J=7.66 Hz, 1 H, 6-H), 4.15 (m, 1 H, 3′-H), 2.91 (m, 1 H, 4′-H), 2.86 (m, 1 H, 5′a-H), 2.77 (m, 1 H, 5′b-H).

Derivative 11

¹H-NMR of derivative 11: (500 MHz, DMSO-d₆, 25° C.): δ=11.03 (s, 1 H, 7-NH), 8.68 (s, 1 H, 12-H), 8.65 (s, 1 H, 19-H), 8.28 (d, J=7.60 Hz, 1 H, 6-H), 7.78 (d, J=8.50 Hz, 1 H, 23-H), 7.15 (d, J=7.60 Hz, 1 H, 5-H), 6.94 (dd, J =8.50, 2.20 Hz, 1 H, 22-H), 6.88 (d, J=2.20 Hz, 1 H, 20-H), 6.35 (d, J=6.50 Hz, 1 H, 3′-OH), 6.19 (t, J=7.50 Hz, 1 H, 1′-H), 5.39 (s, 2 H, 10-H), 5.34 (t, J=5.50 Hz, 5′-OH), 4.22 (m, 1 H, 3′-H), 2.92 (m, 1 H, 4′-H), 2.84 (m, 1 H, 5′a-H), 2.69 (m, 1 H, 5′b-H) ppm.

¹³C-NMR of derivative 11: (500 MHz, DMSO-d₆, 25° C.): δ=164.30 (C4), 162.58 (C2′), 156.77 (C15), 155.61 (C17), 154.76 (C2), 152.71 (C8), 145.51 (C6), 142.68 (C11), 137.29 (C19), 131.76 (C23), 126.85 (C12), 120.12 (C14), 115.1 (C22), 111.17 (C18), 102.91 (C20), 95.65 (C5), 84.81 (C1′), 81.69 (C4′), 69.07 (C3′), 59.49 (C5′), 58.91 (C10) ppm.

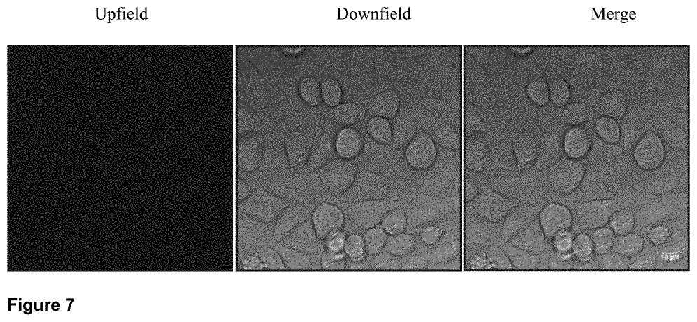

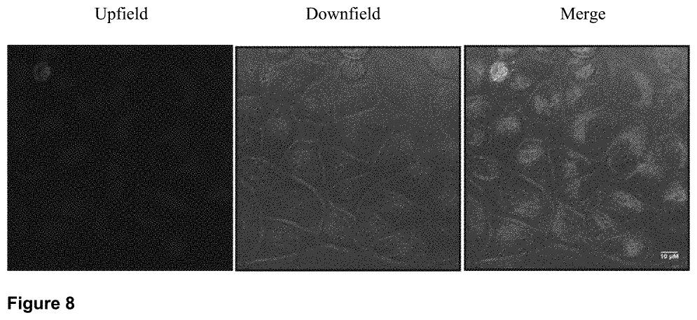

FIG. 7. Results from the confocal microscopy experiments of derivative 11 in HeLa cells. FIG. 7 indicates that derivative 11 does not enter the cells due to its highly polar character. The inventors then acetylated derivative 11 to increase its lipophilic character. The same experiment was conducted with the acetylated form. The results are shown in FIG. 8.

Derivative 12

¹H-NMR of derivative 12: (500 MHz, DMSO-d₆, 25° C.): δ=11.07 (s, 1H, 7-NH), 8.75 (s, 1H, 12-H), 8.67 (s, 1H, 19-H), 8.09 (d, J=7.70 Hz, 1 H, 6-H), 7.99 (d, J=8.50 Hz, 1 H, 23-H), 7.30 (d, J=7.60 Hz, 1H, 5-H), 7.30 (d, J=8.55 Hz, 1 H, 22-H), 6.32 (t, J=8.55 Hz, 1H, 1′-H), 5.46 (m, 1H, 3′-H), 5.26 (s, 2H, 10-H), 3.41 (m, 1H, 4′-H), 3.40 (m, 1H, 5′a-H), 3.35 (m, 1H, 5′b-H), 2.16 (s, 3H, 11′-H), 2.12 (s, 3H, 26-H), 2.07 (s, 3H, 8′-H) ppm.

¹³C-NMR of derivative 12: (500 MHz, DMSO-d₆, 25° C.): δ=163.30 (C4), 163.58 (C2′), 156.77 (C15), 155.61 (C17), 153.76 (C2), 153.71 (C8),135.6 (C12), 126.9 (C19), 147.2(C6), 131.5 (C23), 120.7 (C5), 97.1 (C22), 86.2 (C1′), 71.6 (C3′), 57.8 (C10), 76.7 (C4′), 63.1 (C5′), 21.1 (C11′), 25.2 (C26), 21.6 (C8′) ppm.

FIG. 8. Results from the confocal microscopy experiments of derivative 12 in HeLa cells. FIG. 8 shows a fluorescence signal inside the cells. This means that the acetylation of derivative 11 increased its ability to enter the cells and, simultaneously, that the acetyl groups are cleaved. Without wishing to be bound by theory, the deacetylation was carried out by esterase enzymes which are usually found to be in higher levels in cancer cells (compared to non-cancer cells). The present inventors believe that derivative 12 has a theragnostic character (therapy and diagnosis). The fluorescence upon acetylation of the phenolic hydroxyl group is quenched due to disturbance of the ICT (Internal Charge Transfer) mechanism. Upon the cleavage by the esterase enzymes the fluorescence is restored as is confirmed by the confocal experiments.

Derivative 13

¹H-NMR of derivative 13: (500 MHz, DMSO-d₆, 25° C.): δ=10.88 (s, 1H, 7-NH), 8.06 (d, J=7.84 Hz, 1H, 6-H), 7.15 (d, J=7.51 Hz, 1H, 5-H), 6.30 (t, J=16.14 Hz, 1H, 1′-H), 5,44(m, 1H, 3′-H), 3.45(m, 1H, 4′-H), 3.37(m, 1H, 5′a-H), 3.34(m, 1H, 5′b-H), 3.16 (q, J=21.2 Hz, 2H, 10-H), 2.16(s, 3H, 11′), 2.05(s, 3H, 8′), 1.23 (s, 3H, 11-H).

¹³C-NMR of derivative 13: (500 MHz, DMSO-d₆, 25° C.): δ=163.3 (C4), 153.99 (C2), 153.04 (C8), 145.19 (C2′), 147.5 (C6), 96.5 (C5), 85.9 (C1′), 71.6 (C3′), 76.7(C4′), 62.9(C5′), 62.12 (C10), 20.9 (C11′), 21.25 (C8′), 15(C11).

Mass spectrum of derivative 13: MS (ESI-) m/z: [M-H]− for C₁₆H₁₉F₂N₃O₈: calculated 419.34 found, 418.11, [M+Cl−] C₁₆H₁₉F₂N₃O₈Cl: calculated, 453.30 found, 452.07.

Derivative 14

1H-NMR of derivative 14: (500 MHz, DMSO-d₆, 25° C.): δ=10.89 (s, 1H, 7-NH), 8.05 (d, J=7.71 Hz, 1H, 6-H), 7.14 (d, J=7.35 Hz, 1H, 5-H), 6.30 (t, J=15.96 Hz, 1H, 1′-H), 5.45 (m, 1H, 3′-H), 3.43 (m, 1H, 4′-H), 3.40 (m, 1H, 5′a-H), 3.33(m, 1H, 5′b-H), 3.12 (t, J=13.27 Hz, 2H, 10-H), 2.16 (s, 3H, 11′-H), 2.06 (s, 3H, 8′-H), 1.59 (m, 2H, 11-H), 1.35 (m, 2H, 12-H), 0.9 (t, J=13.34 Hz, 3H, 13-H).

¹³C-NMR of derivative 14: (500 MHz, DMSO-d₆, 25° C.): δ=163.38 (C4), 153.88 (C2), 153.18 (C8) 123.9 (C2′), 146.5 (C6), 95.6 (C5), 85.8 (C1′), 71.3 (C3′), 76.2 (C4′), 63.0 (C5′), 65.6 (C10), 21.1 (C11′), 21.3 (C8′), 30.9 (C11), 19.14 (C12), 13.19 (C13).

Mass of derivative 14: MS (ES1+) m/z: [M+H+] for C₁₈H₂₃F₂N₃O₈: calculated 447.39 found, 448.8, [M+Na+] for C₁₈H₂₃F₂N₃O₈Na: calculated, 470.37 found, 470.7, [M+K+] for C₁₈H₂₃F₂N₃O₈K: calculated, 486.35 found 486.8.

Derivative 15

1H-NMR of derivative 15: (500 MHz, DMSO-d₆, 25° C.): δ=11.14 (s, 1H, 7-NH), 8.14 (d, J=7.37 Hz, 1H, 6-H), 7.18 (d, J=7.55 Hz, 1H, 5-H), 6.38 (t, J=16.8 Hz, 1H, 1′-H), 5.50 (m, 1H, 3′-H), 3.44 (m, 1H, 4′-H), 3.41 (m, 1H, 5′a-H), 3.36 (m, 1H, 5′b-H), 3.39 (m, 2H, 10-H), 3.85 (t, J=10.25 Hz, 11-H), 2.17 (s, 3H, 11′-H), 2.08 (s, 3H, 8′-H).

¹³C-NMR of derivative 15: (500 MHz, DMSO-d₆, 25° C.): δ=163.3 (C4), 153.82 (C2), 153.68 (C8), 129.89 (C2′146.5 (C6), 96.3 (C5), 85.9 (C1′), 71 (C3′), 77 (C4′), 63.2(C5′), 66 (C10), 42.9 (C11), 20.9 (C11′), 21.4 (C8′).

Mass spectrum of derivative 15: MS (ESI-) m/z: [M-H-] for C₁₆H₁₈CIF₂N₃O₈: calculated 452.08 found, 452.07, [M+Cl−] for C₁₆H₁₈C1F₂N₃O₈Cl: calculated, 488.04 found, 488.04.

Derivative 16

¹H-NMR of derivative 16: (500 MHz, DMSO-d₆, 25° C.): δ=8.09 (d, J=7.92 Hz, 1H, 6-H), 7.38 (d, J=7.65 Hz, 1H, 5-H), 6.35 (t, J=16.67 Hz, 1H, 1′-H), 5.44 (m, 1H, 3′), 3.45 (m, 2H, 11-H), 3.41 (m, 1H, 4′-H), 3.40 (m, 1H, 5′a-H), 3.34 (m, 1H, 5′b-H), 3.07 (t, J=15.08 Hz, 2H, 10-H), 2.17 (s, 3H, 11′-H), 2.07 (s, 3H, 8′-H).

¹³C-NMR of derivative 16: (500 MHz, DMSO-d₆, 25° C.): 5=162.0 (C4), 155.03 (C8), 153.82 (C2), 123.89 (C2′) 145.9 (C6), 93.9 (C5), 85.4 (C1′), 71 (C3′), 62.6 (C11), 76.4 (C4′), 62.3 (C5′), 43.1 (C10), 20.9 (C11′), 20.2 (C8′).

Mass of derivative 16: MS (ESI+) m/z: [M+Na⁺] for C₁₆H₁₇F₂N₃O₈Na: calculated, 440.3 found, 440.08, [2M+Na+] for [2C₁₆H₁₇F₂N₃O₈] Na: calculated, 857.62 found 857.18.

Derivative 17

1H-NMR of derivative 17: (500 MHz, DMSO-d₆, 25° C.): δ=11.46 (s, 1H, 7NH), 8.13 (d, J=7.60 Hz, 1H, 6-H), 7.12 (d, J=7. 47 Hz, 1H, 5-H), 6.32 (t, J=17.36 Hz, 1H, 1′-H), 5, 96 (s, 2H, 10-H), 5.44 (m, 1H, 3′H), 3.45 (m, 1H, 4′-H), 3.40 (m, 1H, 5′a-H), 3.36 (m, 1H, 5′b-H), 2.17 (s, 3H, 11′-H), 2.07 (s, 3H, 8′-H) ppm.

Derivative 18

¹H-NMR of derivative 18: (500 MHz, DMSO-d₆, 25° C.): δ=11.12 (s, 1H, 7-NH), 8.08 (d, J=7.65 Hz, 1H, 6-H), 7.12 (d, J=7.21 Hz, 1H, 5-H), 6.30 (t, J=16.62 Hz, 1H, 1′H), 5.43 (m, 1H, 3′-H), 3.79 (d, J=2.65 Hz, 2H, 10-H), 3.45 (m, 1H, 4′), 3.41 (m, 1H, 5′a-H), 3.34 (m, 1H, 5′b-H), 3.6 (t, J=3.70 Hz), 2.16 (s, 3H, 11′-H), 2.06 (s, 3H, 8′-H).

¹³C-NMR of derivative 18: (500 MHz, DMSO-d₆, 25° C.): δ=163.20 (C4), 153.81 (C2), 153.31 (C8), 123.93 (C2′), 79.17 (C11), 149.9 (C6), 96.8 (C5), 86.6 (C1′), 71.4 (C3′), 54 (C10), 76.6 (C4′), 63.4 (C5′), 78.9 (C12), 20.7 (C11′), 20.8 (C8′).

Mass spectrum of derivative 18: MS (ESI+) m/z: [M+H+] for C₁₇H₁₇F₂N₃O₈: calculated 430.33 found, 430.8, [M+Na+] for C₁₇H₁₇F₂N₃O₈Na: calculated, 452.3 found, 452.6.

Derivative 19

¹H-NMR of derivative 19: (500 MHz, DMSO-d₆, 25° C.): δ=10.93 (s, 1H, 7-NH), 8.04 (d, J=7.88 Hz, 1H, 6-H), 7.84 (d, J=7.94 Hz, 1H, 15-H), 7.77 (d, J=7.44 Hz, 1H, 18-H), 7.69 (t, J=15.37 Hz, 1H, 16-H), 7.48 (t, J=13.38 Hz, 1H, 17-H), 7.06 (d, J=7.89 Hz, 5-H), 6.31 (t, J=15.72 Hz, 1′-H), 5.43 (m, 1H, 3′H), 3.45 (m, 1H, 4′-H), 3.40 (m, 1H, 5′a-H), 3.33 (m, 1H, 5′b-H), 3.32 (m, 2H, 10-H), 3.51 (q, J=17.64 Hz, 1H, 11-H), 2.16 (s, 3H, 11′-H), 2.06 (s, 3H, 8′-H), 1.31 (d, J=7.3 Hz, 1H, 12-H).

¹³C-NMR of derivative 19: (500 MHz, DMSO-d₆, 25° C.): δ=163.17 (C4), 153.78 (C2), 153.86 (C8) 137.31 (C13), 123.46 (C14), 123.74 (C2′), 146.8 (C6), 123.51 (C15), 129.43 (C18), 133.65 (C16), 128.38 (C17), 95.7 (C5), 85.2 (C1′), 71.3 (C3′), 76.3 (C4′), 63.2 (C5′), 33.3 (C11), 21.1 (C11′), 21.5 (C8′), 18.5 (C12).

Derivative 20

¹H-NMR of derivative 20: (500 MHz, DMSO-d₆, 25° C.): δ=11.33 (s, 1H, 7-NH), 8.11 (d, J=7.91, 1H, 6-H), 7.74 (s, 1H, 13-H), 7.40 (s, 1H, 16-H), 7.16 (d, J-7.19 Hz, 1H, 5-H), 6.33 (t, J=16.41 Hz, 1H, 1′-H), 5.53 (s, 2H, 10-H), 5.45 (m, 1H, 3′-H), 3.45 (m, 1H, 4′-H), 3.40 (m, 1H, 5′a-H), 3.34 (m, 1H, 5′b-H), 3.96 (s, 3H, 18-H), 3.88 (s, 3H, 20-H), 2.17 (s, 3H, 11′-H), 2.07 (s, 3H, 8′-H).

¹³C-NMR of derivative 20: (500 MHz, DMSO-d₆, 25° C.): δ=163.26 (C4), 153.8 (C2), 153.52 (C15), 153.42 (C8), 148.64 (C14) 139.84 (C12), 127.52 (C11), 123.9 (C2′), 147.3 (C6), 109.1 (C13), 111.3 (C16), 95.9 (C5), 85.7 (C1′), 63.2 (C10), 71.5 (C3′), 76.6 (C4′), 63.1 (C5′), 57.1 (C18), 56.8 (C20), 20.8 (C11′), 21.23 (C8′).

Mass spectrum of derivative 20: MS (ESI+) m/z: [M+H+] for C₂₃H₂₄F₂N₄O₁₂: calculated 587.46 found, 587.14, [2M+H+] for 2[C₂₃H₂₄F₂N₄O₁₂]: calculated 1173.92 found, 1173.28 [2M+K+] for 2[C₂₃H₂₄F₂N₄O₁₂] K: calculated 1211.88 found, 1211.23.

Derivative 21

¹H-NMR of derivative 21: (500 MHz, DMSO-d₆, 25° C.): δ=10.92 (s, 1H, 7-NH), 8.06 (d, J=7.31 Hz, 6-H), 7.14 (d, J=7.76 Hz, 1H, 5-H), 6.30 (t, J=16.44 Hz, 1H, 1′-H), 5.44 (m, 1H, 3′-H), 3.44 (m, 1H, 4′-H), 3.40 (m, 1H, 5′a-H), 3.34 (m, 1H, 5′b-H), 3.91 (d, J=6.85 Hz, 2H, 10-H), 2.16 (s, 3H, 11′-H), 2.06 (s, 3H, 8′-H), 1.90 (m, 1H, 11-H), 0.91 (d, J=6.6 Hz, 6H, 12,13-H).

Derivative 22

¹H-NMR of derivative 22: (500 MHz, DMSO-d₆, 25° C.): δ=11.05 (s, 1H, 7-NH), 8.03 (d, J=7.74 Hz, 1H, 6-H), 7.32 (d, J=7.75 Hz, 1H, 5-H), 6.31 (t, J=16.59 Hz, 1H, 1′-H), 5.45 (m, 1H, 3′), 3.44 (m, 1H, 4′-H), 3.4 (m, 1H, 5′a-H), 3.34 (m, 1H, 5′b-H), 2.16 (s, 3H, 11′-H), 2.12 (s, 3H, 9-H), 2.06 (s, 3H, 8′-H).

¹³C-NMR of derivative 22: (500 MHz, DMSO-d₆, 25° C.): δ=146.7 (C6), 96.8 (C5), 85.7 (C1′), 71.1 (C3′), 76.5 (C4′), 63.1 (C5′), 25.2(C9), 21.2 (C11′), 21.4 (C8′).

### Evaluation of Biological Activity

Biological activities of the Gemcitabine derivative (which may be referred to as analogues) described above were evaluated with two assays:
1. Cell growth assay using the SRB assay in cell lines A549/WT, SW1573/WT, PANC-01 and BXPC-3.
2. MTT assay in human bladder cancer cell line T-24

1. Cell growth assay

The following cell lines were tested:
A549/WT —wild type human lung adenocarcinoma cell line (1)
SW1573/WT —wild type non-small lung human cell line (2),
PANC-01—human pancreatic cancer cell line (3)
BXPC-3 -epithelial human pancreatic adenocarcinoma cells (4).

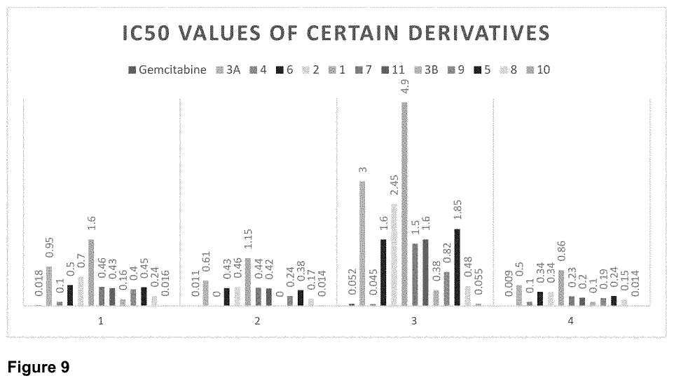

FIG. 9. IC₅₀ values of 4-(N)-acyl and 4-N-phosphoryl derivatives in four different cell lines.

Table 4. IC₅₀ (μM) values of certain Gemcitabine 4-N-acyl and 4-N-phosphoryl derivatives in four different cell lines.

TABLE 4
IC₅₀ (μM) values of certain Gemcitabine 4-N-acyl and 4-N-phosphoryl
derivatives in four different cell lines.

| | | IC₅₀ (μM) | | |
|---|---|---|---|---|
| Derivative | A549/WT | SW 1573/WT | PANC-01 | BXPC-3 |
| Gemcitabine | 0.018 | 0.011 | 0.052 | 0.009 |
| 3A | 0.95 | 0.61 | 3 | 0.5 |
| 4 | 0.1 | 0.1 | 0.045 | 0.1 |
| 6 | 0.5 | 0.43 | 1.6 | 0.34 |
| 2 | 0.7 | 0.46 | 2.45 | 0.34 |
| 1 | 1.6 | 1.15 | 3.9 | 0.86 |
| 7 | 0.46 | 0.44 | 1.5 | 0.23 |
| 11 | 0.43 | 0.42 | 1.6 | 0.2 |
| 3B | 0.16 | 0.1 | 0.38 | 0.1 |
| 9 | 0.4 | 0.24 | 0.82 | 0.19 |
| 5 | 0.45 | 0.38 | 1.85 | 0.24 |
| 8 | 0.24 | 0.17 | 0.48 | 0.15 |
| 10 | 0.016 | 0.014 | 0.055 | 0.014 |

Dipyridamole inhibits adenosine uptake by erythrocytes platelets and endothelial cells in vitro and in vivo. Thus, the present inventors recruited dipyridamole as an inhibitor of the nucleoside transporters of the tumour cells to observe the behaviour of the presently described Gemcitabine derivatives.

From the IC₅₀ of each compound in the presence of dipyridamole, by dividing it with the IC₅₀ of each compound in the absence of dipyridamole, the present inventors calculated a ratio that compared the efficacy of the analogous presence of dipyridamole.

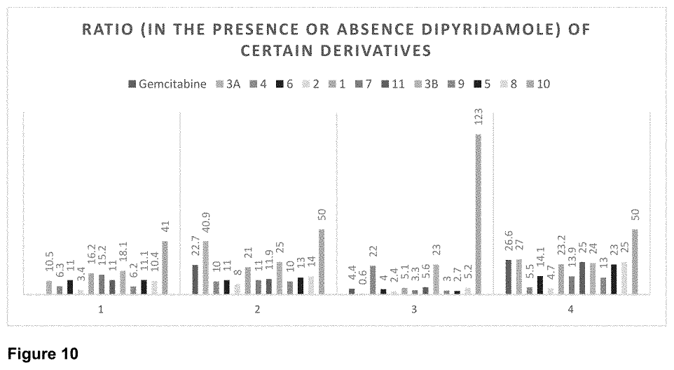

Ratio=IC₅₀ in the presence of dipyridamole/IC₅₀ in the absence of dipyridamole
FIG. 10. Ratio of IC₅₀ of 4-N-acyl and 4-N-phosphoryl derivatives in the presence of, compared to in the absence of, dipyridamole, in four cell lines

Table 5. Values of the ratio of IC₅₀ of 4-N-acyl and 4-N-phosphoryl derivatives in the presence of, compared to in the absence of, dipyridamole, in four cell lines

TABLE 5
Values of the ratio of IC₅₀ of 4-N-acyl and 4-N-phosphoryl
derivatives in the presence of, compared to in the absence of,
dipyridamole, in four cell lines

| Derivative | A549/WT | SW 1573/WT | PANC-01 | BXPC-3 |
|---|---|---|---|---|
| Gemcitabine | 15 | 22.7 | 3.4 | 26.6 |
| 3A | 10.5 | 40.9 | 0.6 | 27 |
| 4 | 6.3 | 10 | 22 | 4.5 |
| 6 | 11 | 11 | 4 | 13.1 |
| 2 | 3.4 | 8 | 2.4 | 3.7 |
| 1 | 16.2 | 21 | 4.1 | 23.2 |
| 7 | 14.2 | 11 | 3.3 | 13.9 |
| 11 | 11 | 11.9 | 4.6 | 25 |
| 3B | 18.1 | 25 | 23 | 24 |
| 9 | 6.2 | 10 | 3 | 13 |
| 5 | 11.1 | 13 | 2.7 | 23 |
| 8 | 10.4 | 14 | 4.2 | 25 |
| 10 | 41 | 50 | 123 | 50 |

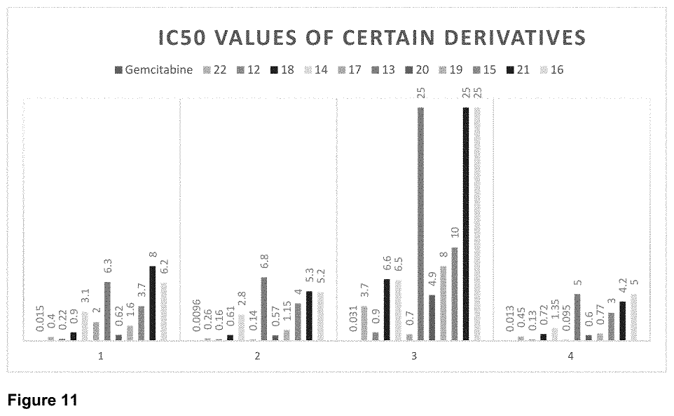

FIG. 11. IC₅₀ values of the acetylated 4-N-acyl derivatives in four cell lines.

Table 6. The IC₅₀ (pM) values of the acetylated 4-N-acyl derivatives in four cell lines.

TABLE 6
The IC₅₀ (μM) values of the acetylated 4-N-acyl derivatives in
four cell lines.

| Derivative | A549/WT | SW 1573/WT | PANC-01 | BXPC-3 |
|---|---|---|---|---|
| Gemcitabine | 0.015 | 0.0096 | 0.031 | 0.013 |
| 22 | 0.4 | 0.26 | 3.7 | 0.45 |
| 12 | 0.22 | 0.16 | 0.9 | 0.13 |
| 18 | 0.9 | 0.61 | 6.6 | 0.72 |
| 14 | 3.1 | 2.8 | 6.5 | 1.35 |
| 17 | 2 | 0.14 | 0.7 | 0.095 |
| 13 | 6.3 | 6.8 | 25 | 5 |
| 20 | 0.62 | 0.57 | 3.9 | 0.6 |
| 19 | 1.6 | 1.15 | 8 | 0.77 |
| 15 | 3.7 | 4 | 10 | 3 |
| 21 | 8 | 4.3 | 25 | 3.2 |
| 16 | 6.2 | 4.2 | 25 | 5 |

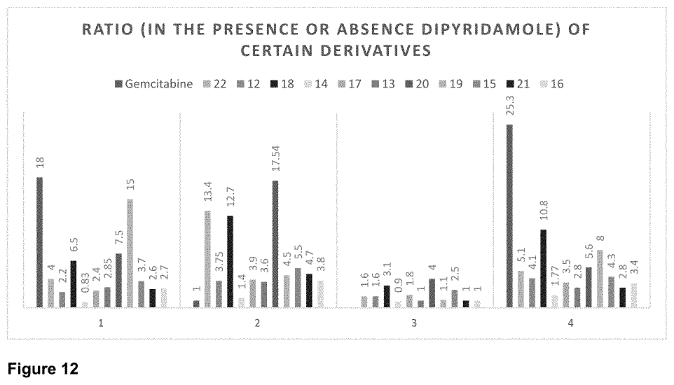

FIG. 12. Ratio of IC₅₀ of the acetylated 4-N-acyl derivatives in the presence of, compared to in the absence of, dipyridamole, in four cell lines.

Table 7. Values of the ratio of IC₅₀ of the acetylated 4-N-acyl derivatives in the presence of, compared to in the absence of, dipyridamole, in four cell lines.

TABLE 7
Values of the ratio of IC₅₀ of the acetylated 4-N-acyl derivatives in
the presence of, compared to in the absence of, dipyridamole,
in four cell lines.

| Derivative | A549/WT | SW 1573/WT | PANC-01 | BXPC-3 |
|---|---|---|---|---|
| Gemcitabine | 18 | 1 | 7.4 | 24.3 |
| 22 | 4 | 13.4 | 1.6 | 4.1 |
| 12 | 2.2 | 3.75 | 1.6 | 3.1 |
| 18 | 6.5 | 12.7 | 3.1 | 10.8 |
| 14 | 0.83 | 1.4 | 0.9 | 1.77 |
| 17 | 2.4 | 3.9 | 1.8 | 3.5 |
| 13 | 2.85 | 3.6 | 1 | 2.8 |
| 20 | 7.5 | 17.54 | 4 | 4.6 |
| 19 | 15 | 3.5 | 1.1 | 8 |
| 15 | 3.7 | 4.5 | 2.5 | 3.3 |
| 21 | 2.6 | 3.7 | 1 | 2.8 |
| 16 | 2.7 | 3.8 | 1 | 3.4 |

The lower the IC₅₀ value, the more potent the cytotoxic derivative. Moreover, the lower the ratio for dipyridamole, the lower the dependence of the derivative uptake into the cell on nucleoside transporters (NTs).

For the A549/WT cell line, the three more potent cytotoxic non-acetylated derivatives are 10>4>3B with IC₅₀ values of 0.016, 0.1 and 0.16, respectively. In this particular cell line, the Gemcitabine IC₅₀ value is 0.018.

Also, the lower dependence on NTs showed the derivatives 2<9<4 with ratio values 3.4, 6.2 and 6.3 respectively. The ratio value of Gemcitabine is 15.

For the SW 1573/WT cell line, the three more potent cytotoxic non-acetylated derivatives are 10>43B with IC₅₀ values of 0.014, 0.1 and 0.1 respectively. In this particular cell line, the Gemcitabine IC₅₀ value is 0.011.

Also, the lower dependent on NTs showed the derivatives 2<4,9<6,7 with ratio values of 8, 10 and 11, respectively. The ratio value of Gemcitabine is 22.7.

For the PANC-01 cell line, the three most potent cytotoxic non-acetylated derivatives are 4>10>>3B with IC₅₀ values of 0.045, 0.055 and 0.38, respectively. In this particular cell line, the Gemcitabine IC₅₀ value is 0.052.

Also, the lower dependence on NTs showed the derivatives 3A<2<5 with values of 0.6, 2.4 and 2.7 respectively. The Gemcitabine value is 3.4.

For the BXPC-3 cell line the three most potent cytotoxic non-acetylated derivatives are 10>43B with IC₅₀ values of 0.014, 0.1 and 0.1, respectively. In this cell line, the Gemcitabine IC₅₀ value is 0.009.

Also, the lower dependence on NTs showed the derivatives 2<4<9 with values of 3.7, 4.5 and 13, respectively. The value of Gemcitabine is 26.6.

For the A549/WT cell line, the three more potent cytotoxic, acetylated derivatives are 12>22>20 with IC₅₀ values of 0.22, 0.4 and 0.62 respectively. In the particular cell line, the Gemcitabine IC₅₀ value is 0.015.

Also, the lower dependence on NTs showed the derivatives of 14 <12 <17 <21 with ratio values of 0.83, 2.2, 2.4 and 2.6, respectively. The Gemcitabine value is 18.

For the cell line SW 1573/WT, the three most potent cytotoxic acetylated derivatives are 17>12>22 with IC₅₀ values of 0.14, 0.16 and 0.26, respectively. In the particular cell line, the Gemcitabine IC₅₀ value is 0.0096.

Also, the lower dependence on NTs showed the derivatives 14 <19 <17 with ratio values of 1.4, 3.5, and 3.6 respectively. The ratio value of Gemcitabine is 1.

For the PANC-01 cell line, the three most potent cytotoxic acetylated derivatives are 17>12>22 with IC₅₀ values of 0.7, 0.9 and 3.7, respectively. In this cell line the Gemcitabine IC₅₀ value is 0.031.

Also, the lower dependence on NTs showed the derivatives 14<13, 16, 21<19 with ratio values of 0.9, 1, and 1.1, respectively. The Gemcitabine ratio value is 7.4.

For the BXPC-3 cell line, the three most potent cytotoxic acetylated derivatives are 17>12>22 with IC₅₀ values of 0.095, 0.13 and 0.45, respectively. In this cell line, the Gemcitabine IC₅₀ value is 0.013.

Also, the lower dependence on NTs showed the derivatives 14<13, 21<12 with values 1.77, 2.8 and 3.1, respectively. The ratio value of Gemcitabine is 24.3.

2. MTT assay

The cell viability of T-24 bladder cancer cells upon treatment with the Gemcitabine derivatives was evaluated using the MTT assay. T24 cancer cells were cultured in DMEM (Gibco) high glucose supplemented with 10% FBS and 1% Penicillin/Streptomycin (100 U/mL Penicillin and 100 μg/mL Streptomycin), at 37 ° C. in humidified atmosphere of 5% CO₂.

For the MTT assay 5000 or 10000 cells were seeded in triplicates in 96-well plates. Stock solutions of each derivative were prepared in DMSO/EtOH (1:1 v/v). Then, the cells were treated and incubated with 100 μM of each compound for 24 or 48 hours. After the completion of the incubation time, 10 μL of MTT solution (5 mg/ml in PBS buffer) were added in each well and incubated for 4 hrs. Finally, to stop the reaction, the supernatant from each well was removed and 100 μL of stop mix solution (20% SDS in 50% dimethyl formamide in water) were added. The plate remained in darkness for 2 h and the absorbance was measured at 540 nm via a microplate ELISA reader (Awareness Technology Inc.) with a reference at 630 nm. The % cell viability for each compound was calculated relatively to the absorbance of the untreated cells (control).

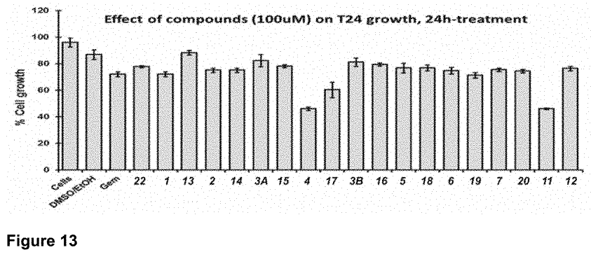

FIG. 13. A plot showing cell viability (%) of T-24 cells (5000 cells/well) treated with 100 μM of Gemcitabine derivatives after 24-hour incubation determined by MTT assay.

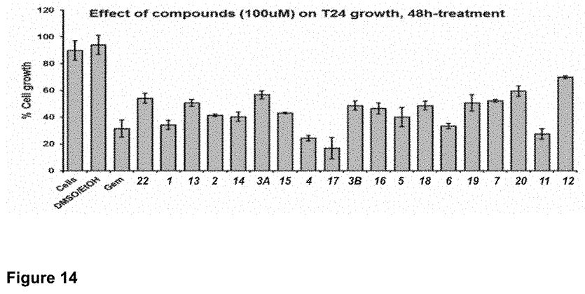

FIG. 14. A plot showing cell viability (%) of T-24 cells (5000 cells/well) treated with 100 μM of Gemcitabine derivatives after 48-hour incubation determined by MTT assay.

Two sets of experiments were performed at concentrations of 100 μM, and the absorption of formazan was measured after 24 and 48 hours.

For the concentration of 100 μM at 24 hours, the present inventors observed a significant inhibition of cell growth, with greater potency than the parent drug Gemcitabine, the order for derivatives was 4>11>17. For the same concentration at 48 hours, the apparent order of potency was 17>4>11 and then Gemcitabine.

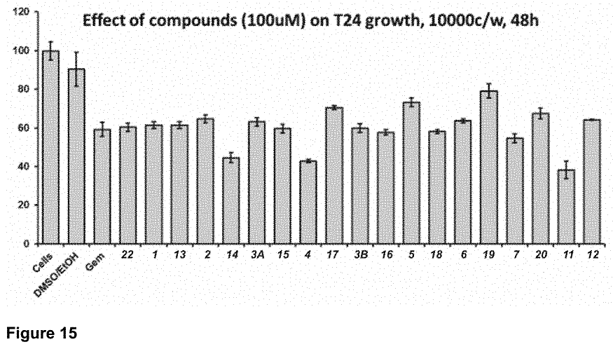

FIG. 15. A plot showing cell viability (%) of T-24 cells (10000 cells/well) treated with 100 pM of Gemcitabine derivatives after 48-hour incubation determined by MTT assay.

Another experiment was conducted with the MTT assay where the compounds were incubated for 48 hours at 100 pM derivative concentration where the number of cells seeded per well was 10000. The most potent derivatives are found to be 14, 4 and 11.

Concentration Dependent Effects of Selected Derivatives

Finally, following the selection of the most potent derivatives the present inventors further investigated the effect of the concentration on the cell viability in the range of 1 to 100 pM concentration. Cells were incubated with the derivatives for 48 h. The selected derivatives were 4, 17 and 11.

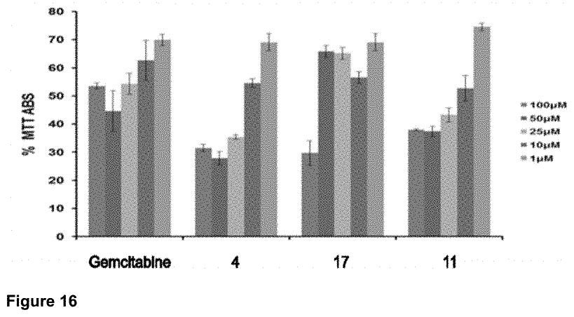

FIG. 16. Cytotoxicity of the most efficient Gemcitabine derivatives at different concentrations in the T-24 cell line.

Human plasma stability of 4-N-ethyl carbamate

Experimental notes:

1. The final concentration of 4-N-ethyl carbamate Gemcitabine in human plasma was 0.1 μM.

2. Each sample was studied in triplicates.

3. The time points during incubation in which the present inventors evaluated the concentration of derivative were: 0, 1, 2, 4, 18 and 24 hours (FIG. 16). The derivative was stable after 24 hours incubation (96% still remaining).

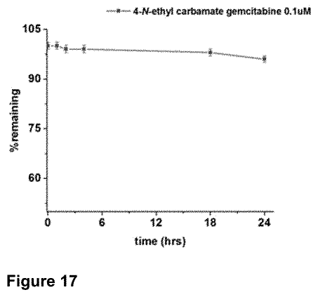

4. A calibration curve was designed in order to quantify the concentration of derivative in human plasma (FIG. 17). Five calibrators were used at concentrations range of 0.01, 0.05, 0.1, 0.2 and 0.4 μM. Coefficient of determination (r²) was calculated 0.999418.

5. Accuracy in terms of trueness and precision was evaluated through the analysis of three replicates at three concentration levels (low: 0.025 μM, mid: 0.08 μM and high: 0.3 μM). The trueness was expressed as the percentage difference between the calculated concentrations and the theoretical prepared concentrations while precision was expressed as CV%.

6. LOQ was determined at 0.01 μM with trueness and precision 8.49% and 9.66% respectively and within the acceptable limits for LOQ (<20%).

7. The intra- and inter-day trueness and precision were found to be 10.2 and 12.7 and within the acceptable limits (<15%).

FIG. 17. In vitro stability of Ethyl-(4-N-Gemcitabine) carbamate (derivative 1) after 24 h incubation in human plasma at 37° C.

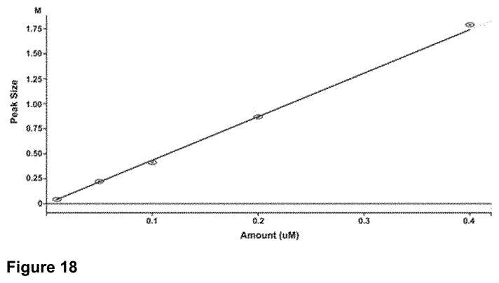

FIG. 18. Calibration curve of Ethyl-(4-N-Gemcitabine) carbamate (derivative 1).

### Conclusions

From the above data, some conclusions can be drawn but also a correlation of activity structure for both 4-(N)-acyl and 4-N-phosphoryl Gemcitabine prodrugs and their 3′, 5′-acetyl derivatives.

Derivatives 10 and 4 in contrast to the non-acetylated derivatives showed significant cytotoxic activity with IC₅₀ values lower than Gemcitabine, in the presence of the adenosine uptake inhibitor, dipyridamole. Therefore, the present inventors have found that the presence of the phosphate and chloromethyl carbamate group in the 4-N position of Gemcitabine increases the action of the drug as it enters cells.

In the presence of dipyridamole, the present inventors found, through the ratio of cytotoxicity, that the profile of activity of the derivatives changed significantly. This is shown at least by the difference in the activity of derivative 10. The ratio value for derivative 10 demonstrated similar action to derivative 2, which carries a n-butyl group, increasing the lipophilic character of the compound. Stable activity gave derivatives having a more lipophilic character, and this is demonstrated by the effects of compound 9 in all cell lines tested, which is the precursor to the relatively hydrophobic molecule derivative 10. Derivative 4 did not have such a high ratio. With derivative 4, a good response was shown to nucleoside transporter suspension; this suggests that the present of the chlorine atom may play a role in combination with the small carbon chain that it possesses.

From the same experiments on the herein disclosed acetylated derivatives, the present inventors believe that increasing lipophilicity helps to improve the profile of Gemcitabine prodrugs. The IC₅₀ values of acetylated bifunctional prodrugs are comparatively like Gemcitabine. The present inventors observe a significant increase of the activity in the presence of dipyridamole, which confirms the above-mentioned view of the increase in lipophilicity and action of the molecule.

The MTT experiments at different concentrations and exposure times supported the above findings for the correlation of the activity of 4-N-acyl prodrugs with:

a) The existence of a short carbon chain at the 4-N site;

b) The existence of a chlorine atom; and,

c) The regulated and not excessive increase in lipophilicity.

The present inventors did not observe the same behaviour in the theragnostic molecule (derivative 12) in this cell line.

When used in this specification and claims, the terms “comprises” and “comprising” and variations thereof mean that the specified features, steps or integers are included. The terms are not to be interpreted to exclude the presence of other features, steps or components.

The results disclosed herein with respect to Gemcitabine derivatives are applicable also to other nucleoside derivatives, for example cytidine derivatives according to formulae (IIIB) and (IIIBP).

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Although certain example embodiments of the invention have been described, the scope of the appended claims is not intended to be limited solely to these embodiments. The claims are to be construed literally, purposively, and/or to encompass equivalents.

1. A method for preparing 4-(N)-protected derivatives of compounds of formula (IB), or a pharmaceutically acceptable salt thereof, the method comprising:
reacting a compound of formula (IB): 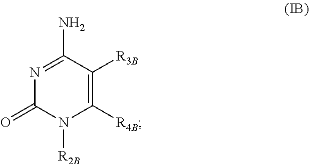
with a chloroformate of the formula (II): 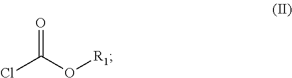
to produce a compound of the formula (IIIB): 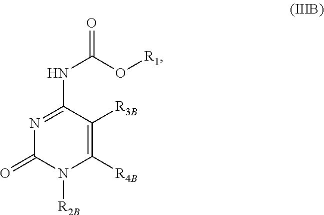
wherein:
R₁ is selected from the group consisting of: substituted or unsubstituted C₁-C₂₆ alkyl, substituted or unsubstituted C₁-C₂₆ haloalkyl, e.g. chloroalkyl, substituted or unsubstituted aryl, substituted or unsubstituted benzyl, substituted or unsubstituted C₂-C₂₆ alkenyl, substituted or unsubstituted C₂-C₂₆ alkynyl, C₁-C₂₆ alkyl substituted with one or more substituted or unsubstituted benzyl groups, C₁-C₂₆ alkyl substituted with one or more substituted or unsubstituted triazole groups;
R_{2B} is selected from the group consisting of: substituted or unsubstituted aromatic ring with 5 carbon atoms, substituted or unsubstituted aromatic ring with 6 carbon atoms, substituted or unsubstituted aryl, substituted or unsubstituted C₁-C₂₆ alkyl, substituted or unsubstituted a pyranose saccharide, substituted or unsubstituted β pyranose saccharide, substituted or unsubstituted α furanose saccharide, or substituted or unsubstituted β furanose saccharide;
R_{3B} is selected from the group consisting of: hydrogen, mono-substituted aromatic ring with 5 atoms, mono-substituted aromatic ring with 6 atoms, di-substituted aromatic ring with 5 atoms, di-substituted aromatic ring with 6 atoms, substituted or unsubstituted aryl, substituted or unsubstituted alkoxyalkane, carbonyl, halogen, substituted or unsubstituted C₁-C₂₆ alkyl, azide, substituted or unsubstituted C₂-C₂₆ alkynyl, substituted or unsubstituted C₂-C₂₆ alkenyl, hydroxyl, amino, or sulfur; and
R_{4B} is selected from the group consisting of: hydrogen, mono-substituted aromatic ring with 5 atoms, mono-substituted aromatic ring with 6 atoms, di-substituted aromatic ring with 5 atoms, di-substituted aromatic ring with 6 atoms, substituted or unsubstituted aryl, substituted or unsubstituted alkoxyalkane, carbonyl, halogen, substituted or unsubstituted C₁-C₂₆ alkyl, azide, substituted or unsubstituted C₂-C₂₆ alkynyl, substituted or unsubstituted C₂-C₂₆ alkenyl, hydroxyl, amino, or sulfur.

2. (canceled)

3. The method of claim 1, wherein R_{2B} is selected from the group consisting of: 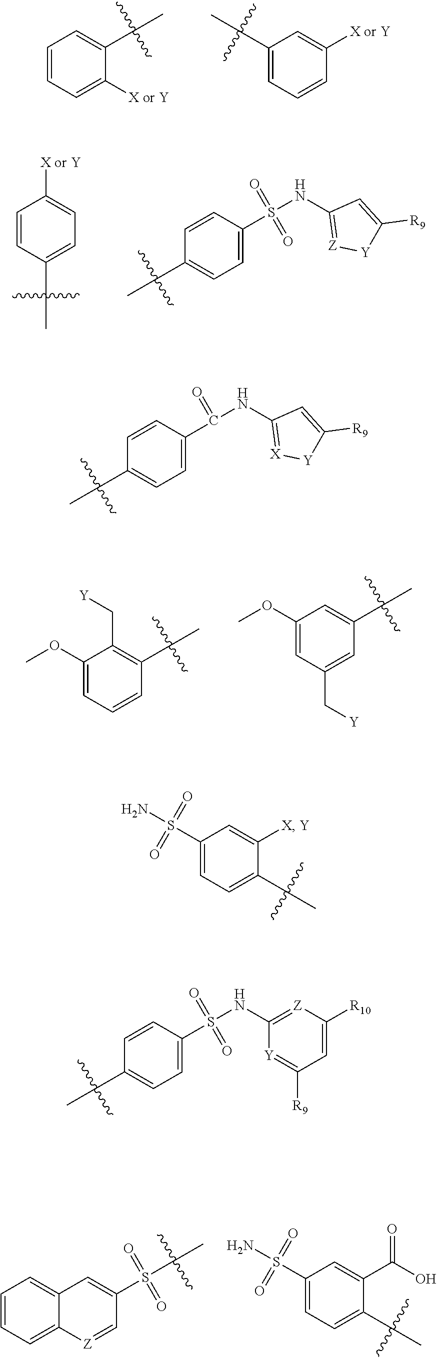 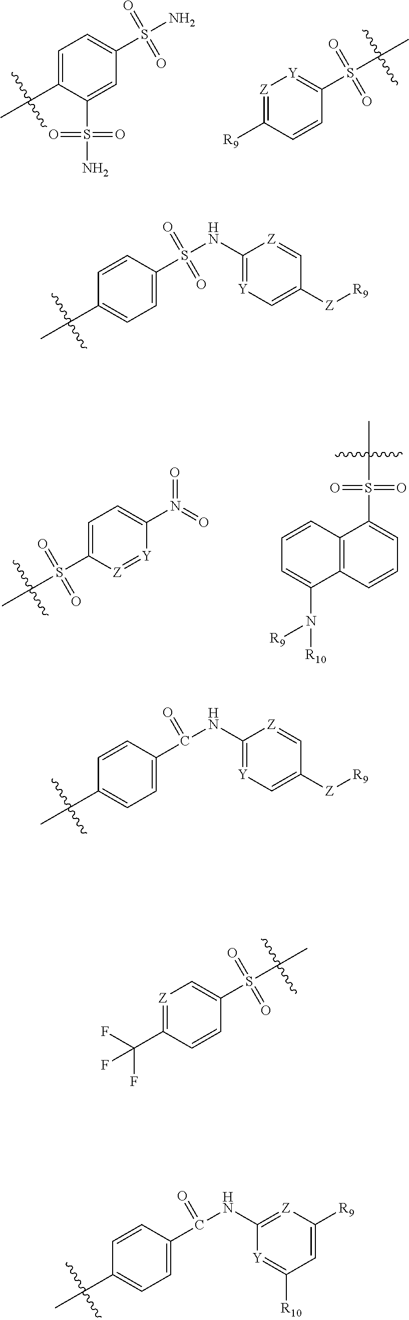 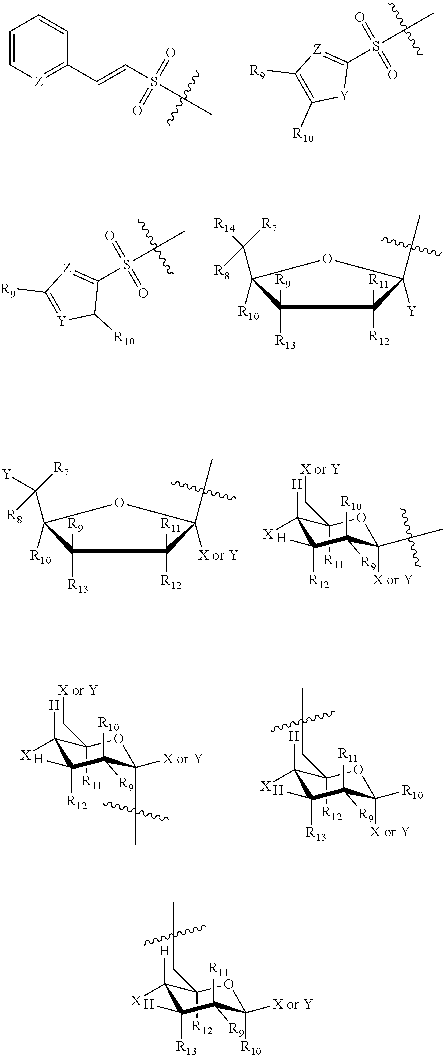
wherein:
the wavy line, at each incidence, shows the point of connection of R_{2B},
R₇ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;
R₈ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;
R₉ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;
R₁₀ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;
R₁₁ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;
R₁₂ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;
R13 is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;
R₁₄ is selected from the group consisting of: alkoxyalkane, carbonyl, halogen, hydrogen, substituted or unsubstituted C₁-C₂₆ alkyl, azide, substituted or unsubstituted C₁-C₂₆ alkynyl, substituted or unsubstituted C₂-C₂₆ alkenyl, hydroxyl, amino, sulfur, or substituted or unsubstituted aryl;
X is independently halogen;
Y is independently hydrogen, hydroxyl, amino or sulfur;
Z is independently hydroxyl, amino or sulfur.

4. The method of claim 1, wherein R_{3B} and R_{4B} are both hydrogen.

5. The method of claim 1, wherein the halogen at each incidence is independently F, Cl, Br or I.

6. The method of claim 1, wherein R_{3B} is hydrogen, R_{4B} is hydrogen, and R_{2B} is

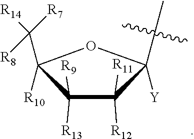

7. The method of claim 6, wherein Y is hydrogen, R₁₁ is halogen, R₁₂ is halogen, R₉ is hydrogen, R₁₃ is hydroxyl (—OH), R₁₀ is hydrogen, R₇ is hydrogen, R₈ is hydrogen and R₁₄ is hydroxyl (—OH).

8. A method for preparing 4-(N)-protected derivatives of Gemcitabine, or a pharmaceutically acceptable salt thereof, the method comprising:
reacting Gemcitabine (I): 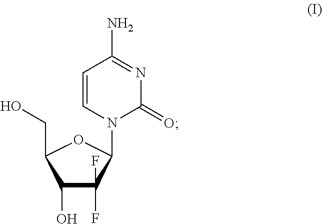
with a chloroformate of the formula (II): 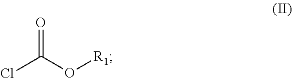
to produce a compound of the formula (III): 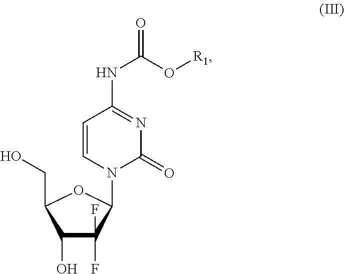
wherein R₁ is selected from the group consisting of: substituted or unsubstituted C₁-C₂₆ alkyl, substituted or unsubstituted C₁-C₂₆ haloalkyl, e.g. chloroalkyl, substituted or unsubstituted C₂-C₂₆ alkenyl, substituted or unsubstituted aryl, substituted or unsubstituted benzyl, substituted or unsubstituted C₂-C₂₆ alkynyl, C₁-C₂₆ alkyl substituted with one or more substituted or unsubstituted benzyl groups, C₁-C₂₆ alkyl substituted with one or more substituted or unsubstituted triazole groups.

9. The method of claim 1, wherein R_{3B} is halogen, R_{4B} is hydrogen, R₁ is —(CH₂)₄CH₃ and R_{2B} is

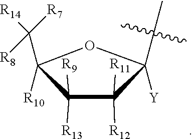

10. The method of claim 9, wherein R_{3B} is F.

11. The method of claim 9 or claim 10, wherein Y is hydrogen, R₁₁ is hydrogen, R₁₂ is hydroxyl (—OH), R₉ is hydrogen, R₁₃ is hydroxyl (—OH), R₁₀ is hydrogen, R₇ is hydrogen, R₈ is hydrogen and R₁₄ is hydrogen.

12. (canceled)

13. The method of claim 1, wherein the method occurs in one pot; optionally, wherein the method occurs in a single step without isolation of an intermediate.

14. The method of claim 1, wherein the chloroformate of the formula (II) or the phosphoryl chloride of the formula (IIP),
is present in the method at from 0.3 to 0.7 equivalents (by moles).

15. The method of claim 1, wherein the acyl chloridcchloroformate of the formula (II) or the phosphoryl chloride of the formula (IIP),
is present in the method at 0.5 equivalents (by moles).

16. The method of claim 1, wherein reacting the compound of formula (IB), optionally Gemcitabine (I), with the acyl chloroformate of formula (II) or the phosphoryl chloride of the formula (IIP) occurs in a solvent of ethyl acetate, acetyl cyanide or a mixture of ethyl acetate and acetyl cyanide.

17. The method of claim 1, wherein reacting the compound of formula (IB), optionally Gemcitabine (I), with the acyl chloroformate of formula (II) or the phosphoryl chloride of the formula (IIP) occurs under reflux conditions for from 1 to 4 hours; optionally for 3 hours; optionally, wherein reflux conditions occur at from 70° C. to 90° C., or at 80° C.

18. The method of claim 1, wherein R₁ is selected from the group consisting of: —CH₂CH₃, —(CH₂)₂CH₃, —(CH₂)₃CH₃, —(CH₂)₄CH₃, —(CH₂)₅CH₃, —(CH₂)₆CH₃, —CH₂CH(CH₃)₂, —(CH₂)₂CH(CH₃)₂, —(CH₂)₃CH(CH₃)₂, —(CH₂)₄CH(CH₃)₂, —CH₂Cl, —(CH₂)₂Cl, —(CH₂)₃Cl, —(CH₂)₄Cl, —(CH₂)₅Cl, —(CH₂)₆Cl, —CH₂Br, —(CH₂)₂Br, —(CH₂)₃Br, —(CH₂)₄Br, —(CH₂)₅Br, —(CH₂)₆Br, —CH₂I, —(CH₂)₂I, —(CH₂)₃I, —(CH₂)₄I, —(CH₂)₅I, —(CH₂)₆I, —CH₂CCH, —(CH₂)₂CCH, —(CH₂)₃CCH, —(CH₂)₄CCH, —(CH₂)₅CCH, —(CH₂)₆CCH, —CH₂N₃, —(CH₂)₂N₃, —(CH₂)₃N₃, —(CH₂)₄N₃, —(CH₂)₅N₃, —(CH₂)₆N₃, —CH₂SH, —(CH₂)₂SH, —(CH₂)₃SH, —(CH₂)₄SH, —(CH₂)₅SH, —(CH₂)₆S—CH₂COOH, —(CH₂)₂COOH, —(CH₂)₃COOH, —(CH₂)₄COOH, —(CH₂)₅COOH, —(CH₂)₆COOH, —CH₂COOR₂, —(CH₂)₂COOR₂, —(CH₂)₃COOR₂, —(CH₂)₄COOR₂, —(CH₂)₅COOR₂, —(CH₂)₆COOR₂, —CH₂Ar, —(CH₂)₂Ar, —(CH₂)₃Ar, —(CH₂)₄Ar, —(CH₂)₅Ar, —(CH₂)₆Ar, —CH₂CHArCH₃, —CH₂CHArCH₂CH₃, —CH₂Tr, —(CH₂)₂Tr, —(CH₂)₃Tr, —(CH₂)₄Tr, —(CH₂)₅Tr, —(CH₂)₆Tr, —CH₂CHTrCH₃ or —CH₂CHTrCH₂CH₃;
wherein R₂ is substituted or unsubstituted C₁-C₂₆ alkyl;
wherein Ar is 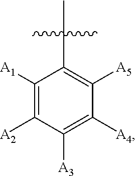
wherein A₁, A₂, A₃, A₄ and A₅ are each independently H, NO₂, OH, O-alkyl or O-methyl; optionally, wherein A₁ is NO₂ and A₂, A₃, A₄ and A₅ are H; or, wherein A₁ is NO₂, A₃ and A₄ are O-methyl and A₂ and A₅ are H; and/or,
wherein Tr is 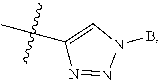
wherein B is substituted or unsubstituted alkyl, substituted or unsubstituted haloalkyl, e.g. chloroalkyl, substituted or unsubstituted aryl, substituted or unsubstituted benzyl, substituted or unsubstituted alkenyl, substituted or unsubstituted alkynyl, alkyl substituted with one or more benzyl or substituted benzyl groups or

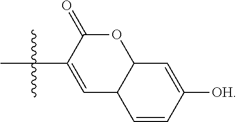 [SMILES: *C1=CC2C=CC(O)=CC2OC1=O]

19. The method of claim 1, wherein R₁ comprises a substituent reactive with the H atom on 4-(N), e.g. wherein R₁ is chloroalkyl and the method further comprises the step of reacting the compound of the formula (III): 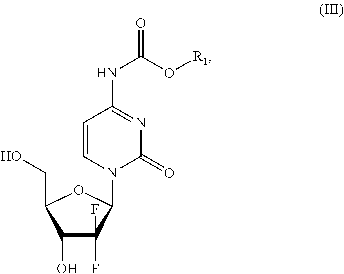
in a solvent, e.g. N,N-diisopropylethylamine, under suitable conditions, e.g. reflux conditions, to form a compound of formula (IV): 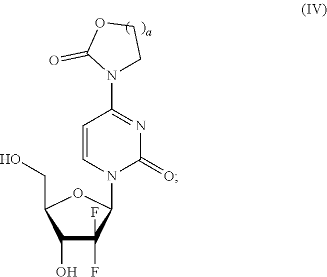
wherein n is 0, 1 or 2.

20. The method of claim 1, wherein
the method further comprises the step of reacting the compound of the formula (III) or (IIP)
with an OH-reactive derivatising agent to form a 3′- and/or 5′-substituted derivative of compound (III) or (IIIP);
optionally, wherein the method further comprises the step of reacting the compound of formula (III) with acetic anhydride to form a compound of the formula (V): 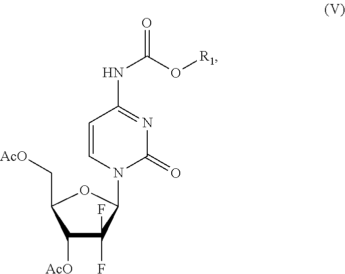
or, formula (VP): 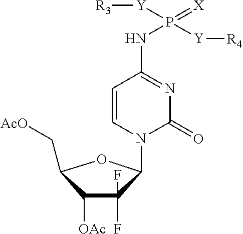
wherein Ac is —COCH₃.

21. A compound obtainable by, or obtained from, the method of claim 1.

22. A compound of the formula (III), or a 3′- and/or 5′-substituted derivative thereof, for example a compound of formula (VA) or (V): 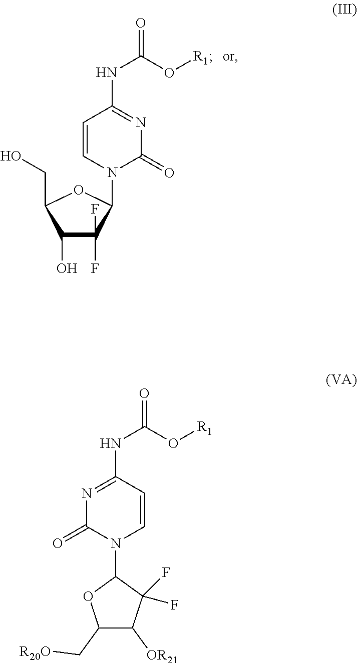
wherein at least one of R₂₀ and R₂₁ is not H, and,
R₂₀ is H or —COR₂₀₁ where R₂₀₁ is selected from the group consisting of: substituted or unsubstituted C₁-C₂₆ alkyl, substituted or unsubstituted C₁-C₂₆ haloalkyl, e.g. chloroalkyl, substituted or unsubstituted aryl, substituted or unsubstituted benzyl, substituted or unsubstituted C₂-C₂₆ alkenyl, substituted or unsubstituted C₂-C₂₆ alkynyl, C₁-C₂₆ alkyl substituted with one or more substituted or unsubstituted benzyl groups, C₁-C₂₆ alkyl substituted with one or more substituted or unsubstituted triazole groups; and,
R₂₁ is H or —COR₂₀₂ where R₂₀₂ is selected from the group consisting of: substituted or unsubstituted C₁-C₂₆ alkyl, substituted or unsubstituted C₁-C₂₆ haloalkyl, e.g. chloroalkyl, substituted or unsubstituted aryl, substituted or unsubstituted benzyl, substituted or unsubstituted C₂-C₂₆ alkenyl, substituted or unsubstituted C₂-C₂₆ alkynyl, C₁-C₂₆ alkyl substituted with one or more substituted or unsubstituted benzyl groups, C₁-C₂₆ alkyl substituted with one or more substituted or unsubstituted triazole groups; or, 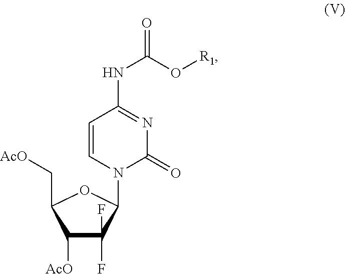
wherein Ac is —COCH₃;
wherein Ri is selected from the group consisting of: substituted or unsubstituted C₁-C₂₆ alkyl, substituted or unsubstituted C₁-C₂₆ haloalkyl, e.g. chloroalkyl, substituted or unsubstituted aryl, substituted or unsubstituted benzyl, substituted or unsubstituted C₂-C₂₆ alkenyl, substituted or unsubstituted C₂-C₂₆ alkynyl, C₁-C₂₆ alkyl substituted with one or more substituted or unsubstituted benzyl groups, C₁-C₂₆ alkyl substituted with one or more substituted or unsubstituted triazole groups;
or a pharmaceutically acceptable salt thereof.

23. The compound of claim 22, wherein R₁ is selected from the group consisting of: —CH₂CH₃, —(CH₂)₂CH₃, —(CH₂)₃CH₃, —(CH₂)₄CH₃, —(CH₂)₅CH₃, —(CH₂)₆CH₃, —CH₂CH(CH₃)₂, —(CH₂)₂CH(CH₃)₂, —(CH₂)₃CH(CH₃)₂ or —(CH₂)₄CH(CH₃)₂.

24. The compound of claim 22, wherein R₁ is selected from the group consisting of: —CH₂Cl, —(CH₂)₂Cl, —(CH₂)₃Cl, —(CH₂)₄Cl, —(CH₂)₅Cl, —(CH₂)₆Cl, —CH₂Br, —(CH₂)₂Br, —(CH₂)₃Br, —(CH₂)₄Br, —(CH₂)₅Br, —(CH₂)₆Br, —CH₂I, —(CH₂)₂I, —(CH₂)₃I, —(CH₂)₄I, —(CH₂)₅I or —(CH₂)₆I.

25. The compound of claim 22, wherein R₁ is selected from the group consisting of: —CH₂CCH, —(CH₂)₂CCH, —(CH₂)₃CCH, —(CH₂)₄CCH, —(CH₂)₅CCH or —(CH₂)₆CCH.

26. The compound of claim 22, wherein R₁ is selected from the group consisting of: —CH₂N₃, —(CH₂)₂N₃, —(CH₂)₃N₃, —(CH₂)₄N₃, —(CH₂)₅N₃ or —(CH₂)₆N₃.

27. The compound of claim 22, wherein R₁ is selected from the group consisting of: —CH₂SH, —(CH₂)₂SH, —(CH₂)₃SH, —(CH₂)₄SH, —(CH₂)₅SH or —(CH₂)₆SH.

28. The compound of claim 22, wherein Ri is selected from the group consisting of: —CH₂COOH, —(CH₂)₂COOH, —(CH₂)₃COOH, —(CH₂)₄COOH, —(CH₂)₅COOH, —(CH₂)₆COOH, —CH₂COOR₂, —(CH₂)₂COOR₂, —(CH₂)₃COOR₂, —(CH₂)₄COOR₂, —(CH₂)₅COOR₂ or —(CH₂)₆COOR₂;
wherein R₂ is substituted or unsubstituted C₁-C₂₆ alkyl.

29. The compound of claim 22, wherein R₁ is selected from the group consisting of: —CH₂Ar, —(CH₂)₂Ar, —(CH₂)₃Ar, —(CH₂)₄Ar, —(CH₂)₅Ar, —(CH₂)₆Ar, —CH₂CHArCH₃ or —CH₂CHArCH₂CH₃;
wherein Ar is 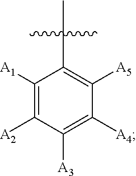
wherein A₁, A₂, A₃, A₄ and A₅ are each independently H, NO₂, OH, O-alkyl or O-methyl; optionally, wherein A₁ is NO₂ and A₂, A₃, A₄ and A₅ are H; or, wherein A₁ is NO₂, A₃ and A₄ are O-methyl and A₂ and A₅ are H.

30. The compound of claim 22, wherein R₁ is selected from the group consisting of: —CH₂Tr, —(CH₂)₂Tr, —(CH₂)₃Tr, —(CH₂)₄Tr, —(CH₂)₅Tr, —(CH₂)₆Tr, —CH₂CHTrCH₃ or —CH₂CHTrCH₂CH₃;
wherein Tr is 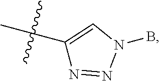
wherein B is substituted or unsubstituted C₁-C₂₆ alkyl, substituted or unsubstituted C₁-C₂₆ haloalkyl, e.g. chloroalkyl, substituted or unsubstituted aryl, substituted or unsubstituted benzyl, substituted or unsubstituted C₂-C₂₆ alkenyl, substituted or unsubstituted C₂-C₂₆ alkynyl, C₁-C₂₆ alkyl substituted with one or more benzyl or substituted benzyl groups, or,

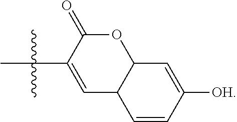 [SMILES: *C1=CC2C=CC(O)=CC2OC1=O]

31. The compound of claim 22, wherein the compound is selected from the group consisting of:

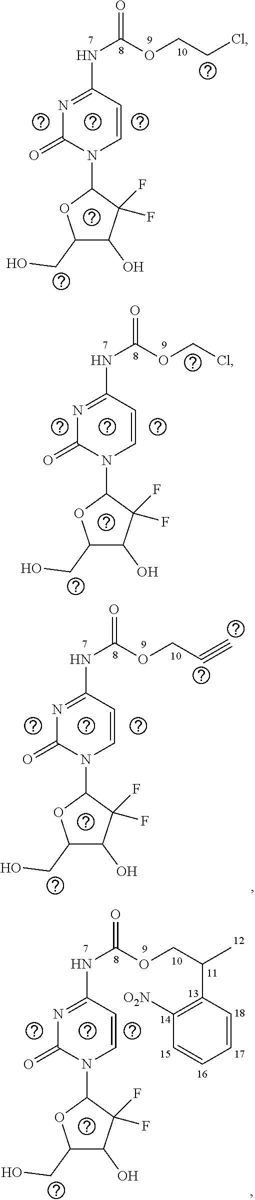

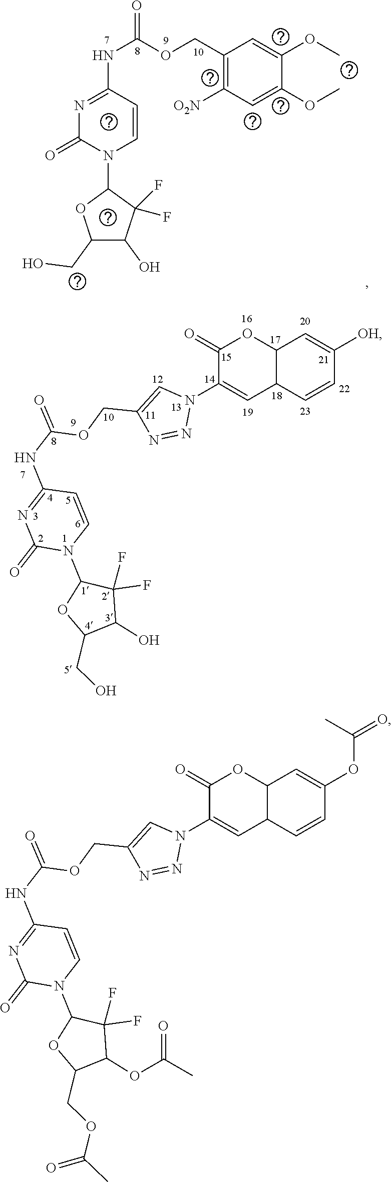 [SMILES: COc1cc(COC(=O)Nc2ccn(C3OC(CO)C(O)C3(F)F)c(=O)n2)c([N+](=O)[O-])cc1OC ; O=C(Nc1ccn(C2OC(CO)C(O)C2(F)F)c(=O)n1)OCc1cn(C2=CC3C=CC(O)=CC3OC2=O)nn1 ; CC(=O)OCC1OC(n2ccc(NC(=O)OCc3cn(C4=CC5C=CC(OC(C)=O)=CC5OC4=O)nn3)nc2=O)C(F)(F)C1OC(C)=O]

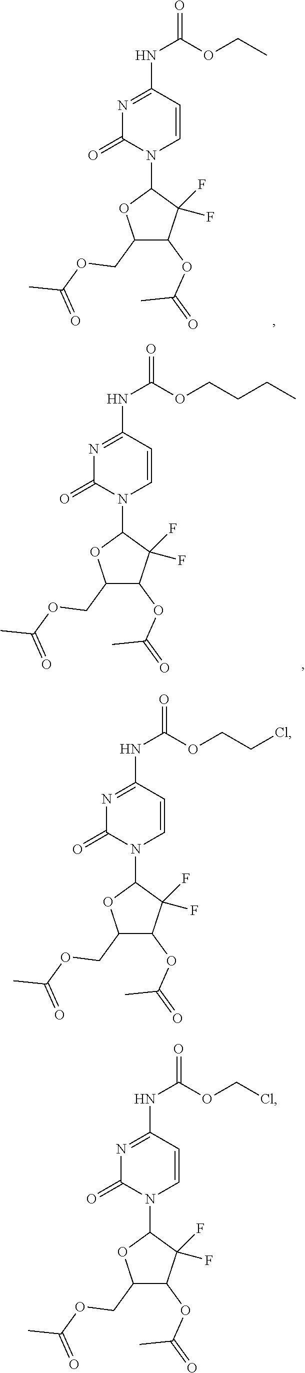

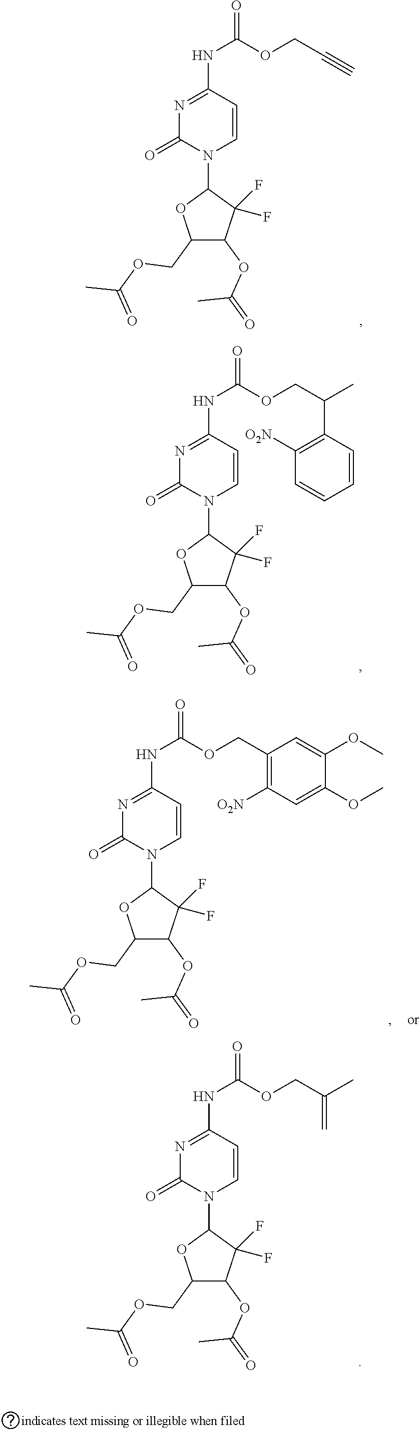

32. The compound of claim 22, wherein the compound is not selected from the group consisting of:

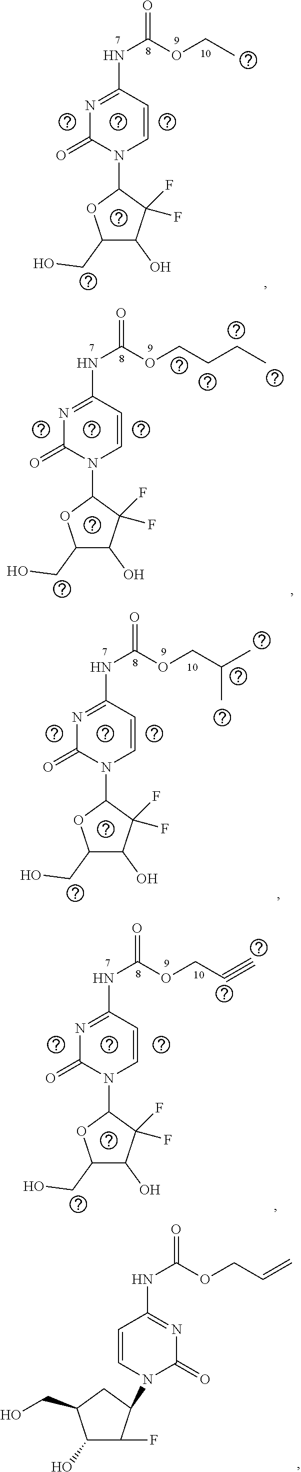

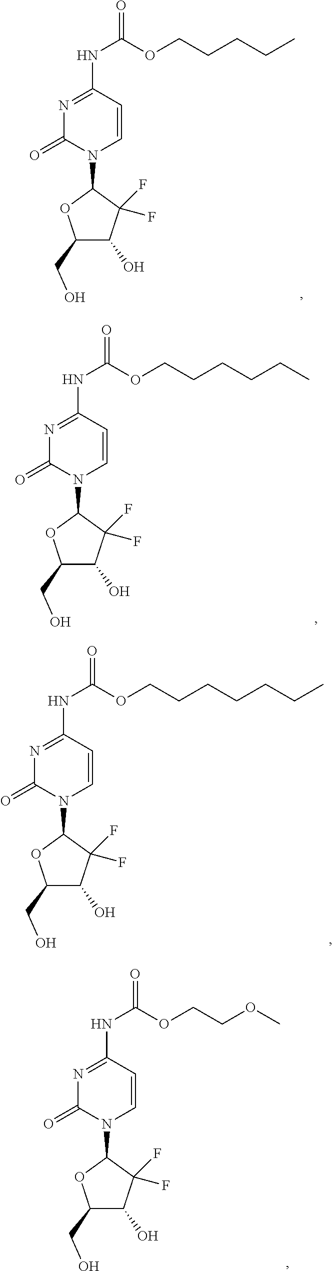 [SMILES: CCCCCOC(=O)Nc1ccn([C@@H]2O[C@H](CO)[C@@H](O)C2(F)F)c(=O)n1 ; CCCCCCOC(=O)Nc1ccn([C@@H]2O[C@H](CO)[C@@H](O)C2(F)F)c(=O)n1 ; CCCCCCCOC(=O)Nc1ccn([C@@H]2O[C@H](CO)[C@@H](O)C2(F)F)c(=O)n1 ; COCCOC(=O)Nc1ccn([C@@H]2O[C@H](CO)[C@@H](O)C2(F)F)c(=O)n1]

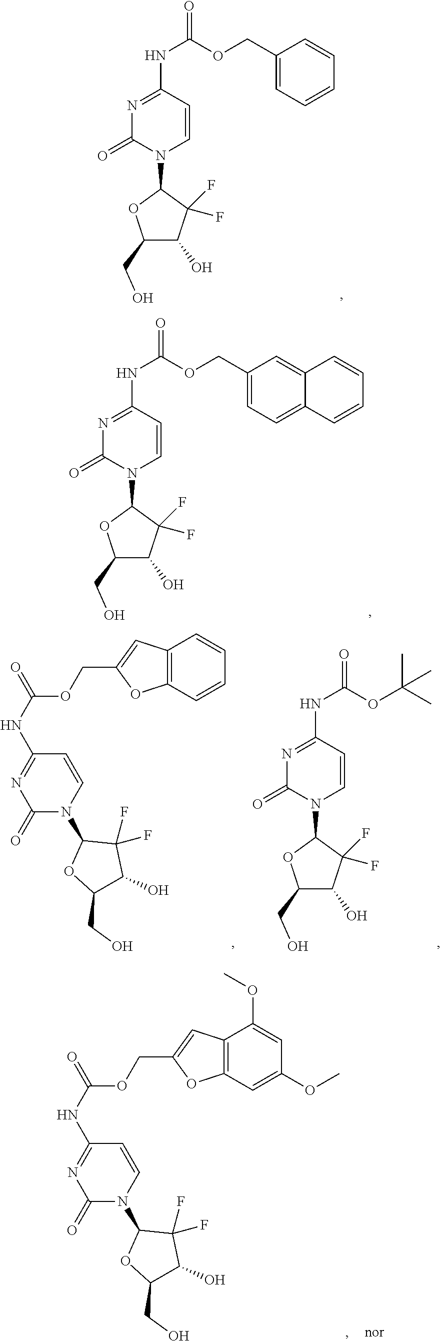

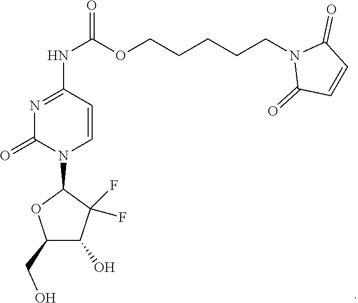 [SMILES: O=C(Nc1ccn([C@@H]2O[C@H](CO)[C@@H](O)C2(F)F)c(=O)n1)OCCCCCN1C(=O)C=CC1=O]

33-35. (canceled)

36. A compound of formula (IV): 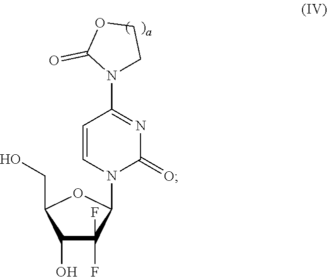
wherein n is 0, 1 or 2;
or a pharmaceutically acceptable salt thereof.

37. A pharmaceutical composition comprising a compound of claim 22 and a pharmaceutically acceptable carrier.

38. A method of treating a disease condition, the method comprising administering to a subject in need thereof a therapeutically effective amount of a compound or claim 22, or a pharmaceutically acceptable salt thereof.

39. A method of treating cancer, the method comprising administering to a subject in need thereof a therapeutically effective amount of a compound or claim 22, or a pharmaceutically acceptable salt thereof.

40. The method of claim 39, wherein the cancer is selected from the group consisting of: breast cancer, ovarian cancer, non-small cell lung cancer, pancreatic cancer and bladder cancer.
